SEQUENCE LISTING (206 sequences)

SEQ ID NO: 1 - DNA, 4223 bp, Zea mays
  misc_feature 1..4223: note = genomic DNA of mutated F35H gene with 8 bp insertion
  source 1..4223: mol_type = genomic DNA
atgcagctcgcggcgttgtgcaccgaccccgtggtgctgtgcagcgccttcctctgcctcctcctccacgtggctctccgctcgctgctgcaccctccttctgcggttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 2 - DNA, 1589 bp, synthetic construct
  misc_feature 1..1589: note = cDNA of mutated F35H gene with 8 bp insertion
  source 1..1589: mol_type = other DNA
atgcagctcgcggcgttgtgcaccgaccccgtggtgctgtgcagcgccttcctctgcctcctcctccacgtggctctccgctcgctgctgcaccctccttctgcggttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcc

SEQ ID NO: 3 - protein, 84 aa, synthetic construct
  REGION 1..84: note = amino acid sequence encoded by SEQ ID NO: 2
MQLAALCTDPVVLCSAFLCLLLHVALRSLLHPPSAVLPPLPPGAAGSSRRGHRACRSSARCHSWAQPRTPAWPRWRASTVPSCT

SEQ ID NO: 4 - DNA, 4222 bp, Zea mays
  misc_feature 1..4222: note = genomic DNA of mutated F35H gene with 7 bp insertion
  source 1..4222: mol_type = genomic DNA
atgcagctcgcggcgttgtgcaccgaccccgtggtgctgtgcagcgccttcctctgcctcctcctccacgtggctctccgctcgctgctgcaccctccttctgcggtctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 5 - DNA, 1588 bp, synthetic construct
  misc_feature 1..1588: note = cDNA of mutated F35H gene with 7 bp insertion
  source 1..1588: mol_type = other DNA
atgcagctcgcggcgttgtgcaccgaccccgtggtgctgtgcagcgccttcctctgcctcctcctccacgtggctctccgctcgctgctgcaccctccttctgcggtctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcc

SEQ ID NO: 6 - protein, 529 aa, synthetic construct
  REGION 1..529: note = amino acid sequence encoded by SEQ ID NO: 5
MQLAALCTDPVVLCSAFLCLLLHVALRSLLHPPSAVCRLFLRAPRAAPAGATGPADPRRAATRGPSPARRPGRAGAQVRSHHVPEDGHGRRGGGVVPARGADVPQGAGRAVRQPAGRGERRGHHVRAAEHGVRGLRAQVEADAEARQRAPARRARARGLGVRAARRGRPRAARRGGGGRGRQARRRAGAARVRPRQHRRADHSEQAGVRRAGGRLERYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 7 - DNA, 4215 bp, Zea mays
  source 1..4215: mol_type = genomic DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 8 - DNA, 1581 bp, synthetic construct
  misc_feature 1..1581: note = cDNA of wildtype F35H gene - genotype PH209
  source 1..1581: mol_type = other DNA
atgcagctcgcggcgttgtgcaccgaccccgtggtgctgtgcagcgccttcctctgcctcctcctccacgtggctctccgctcgctgctgcaccctccttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcc

SEQ ID NO: 9 - protein, 527 aa, Zea mays
MQLAALCTDPVVLCSAFLCLLLHVALRSLLHPPSAASSSGRRGQLPPGPPGLPILGALPLVGPAPHAGLAALARKYGPIMYLKMGTAGVVVASSPRAARTFLKALDARYANRPAVASAADITYGRQNMVFADYGPKWKLMRKLASVHLLGARALADWACVRRGEAGHVLRGVAEAAAAGRPVVVPELLVCALANIVGQITVSKRVFDAQGDDSNRYKDMIVSLLTGTGMFNISDFVPALARLDLQGVQAKLRRVHRQFDGLITKLLAEHAATAADRARQGRPDFVDRLRATMDAGAAADDESGETITEVNIKGLIFDMFTAGTDTSSIIVEWAMAEMLKNPTVMARAQEELDRAVGRGRRLEESDLPGLPYLQAVCKEAMRLHPSTPLSLPHFSLDACDDVDGYRVPANTRLLVNVWAIGRDPEAWERPLDFRPERFLPGGGAEKVDPLGNCFELIPFGAGRRICAGKLAGMVFVQYFLGTLLHAFDWRLPDGEEKLDMSETFGLALPKAVPLRAVATPRLVPEAYA

SEQ ID NO: 10 - DNA, 4402 bp, Zea mays
  misc_feature 1..4402: note = genomic DNA of mutated F35H gene with long insertion
  source 1..4402: mol_type = genomic DNA
atgcagctcgcggcgttgtgcaccgaccccgtggtgctgtgcagcgccttcctctgcctcctcctccacgtggctctccgctcgctgctgcaccctccttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatgcttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacaggtgaggatgggaggtccatgaaatcctaccagctgtgagcatgcataaaagttcatttggaaagaaaagaacatatttttcttacaaatttatgcttactgtttctttaagtttcgataaagtttgtaaaaaaaatttaggctagtttgaaactccatttaggatttctattttccaaagaaaaataaacgaatttctcttgaaaaaatgaaaattctttagaaaaataggttctcaaactagccctcaataaaacttaatgcgatcgttttctctgactctcattcatctttctctggttatctaattgggtccttgagagatgagtttacctgcttgtcctttattattgcaaagacaacatatctgatgcacatggaacattggtgcacatggtgcacatatgaaatcatcaccactcattttaaatctaacgtctatagttgtttgatatattttattaaggacaccctccaacgtggtggtgtgtagtggtggaaggtgttatttgtaaattgaataatcaactagagacgttagatctaaaatgagtggtgatgatttaatatgtgcaccatgtgcaccagtcttctatgtgcaccagatatgtcctctattgcaaatgctagacggaacaccagctagcactagcagactgtttatgtggaaagaaaaaacttaaaaagatcagctaggaagctgctgtcatctgtacgtatatatggtgaagactgaacaatctgcatgacaagcaaaacttagcttaaaagcgaaaagagcgatggaaacggccgctcgataaataattaatgagagtcttgggatttttcatgcatggaaaaaaacaaagctggcatttttcatctaatataatatataacgctgatatcatattgcgtgcagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgtaagctccctgctttttcctcgcccccaaccatgcatcatcatatgcacttatattttacacttgctcggttttcctttagtaactaactaatccgtcgcagctgcgatacacgtagcactagtactacagcgatgggtcatcggtaactgaatctaaggtgcaatagagtgcacggccgcgggatcatggcgtgacatgggagctaagctaagccagtggccacctaacgaaggcactgaccgaaagctcagtggcgtgttaggtggagatagtggatcgagttgttggaaagacaatatcaaaaccactctccaattgatgatgtgtagggcctgcagtgttttgaatccaccttgtttggtcgaacacattactagagtgaaatatggttccaatgttaattgatagcgcgaaagggtctctagcgtaatggttaaaccttccgagtagcacatccaggttgggttcgatcctctcgagggcgaattttcaagctttgttaaaaaaattatctcgttgtgccccgtccgctctcaggaatcgatattctacacgacaccctccgactagtgacagttgattgactcgttagtgatgagaagccatgctaaaaaagtggagacgtagatatgatagaggttccctttcctaagcaaacgtgaatgctatgaaaattatgcagtttaaaaaaaactttaaagataaacaggaattctcttttttggaacaaacaatacgaatgcacctccaaatatcttatcgagtcgacttttatggaattattgtttttgttatttctaagatgggagcccaaaatcacatacaaattattcagtgaatgcctcggtgtttttttattagttaagggctctcatttttttcaagggatttttatttttttccaaaagaaaataaactaatcctctttagaaaaatggaaatctattggagaaatgaggttcctaaactagctctaacagtgagtcagttaatcaggagaagatattagactcctgtatagtgtgcagcaaccacatccgattctgacgttttagcttaatgttcgctatgtagacgtcgggcatagggaatgcattgctaccagaacacgaatgacagctatgcaagtctctagaacgttggagtagttaacaaacgtgatagatgtaacctctggatcatggtatgatgtcatttcctagactagaagaattggtagtcaaatcgagcaaagtcccgaaagcacactgggctttcgacacagtgataccaaagatgctgaaaagaactgaggcacgataaactgttcggtgttggtgtaaacgaccaaagatgctgaaaataactgatcgtcaccatccgtgaatctaactttcgacacactgttaccaaatccttcgtcaaaattacaggaataattaaggcgcttagacgatgataaaccattttttgtcactaattaaccacactgttctttgcttgaccgtgacaaaaaaaaacttttttgtgaagcagtgttgccgtaaaccacaaccatcatgaactcacttgccttgtcatatgtacttgtaccatcgaacgccgcgcgctaagacaatgcaccacccttcaagtcttagctcactgataccgctaattaagttagataatgtcgattactagttgtcttacttcgaactatttcttttcggcaaactgaagtaaagacaacgttttgttccgcaggacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcctga

SEQ ID NO: 11 - DNA, 1768 bp, synthetic construct
  misc_feature 1..1768: note = cDNA of F35H gene with long insertion
  source 1..1768: mol_type = other DNA
atgcagctcgcggcgttgtgcaccgaccccgtggtgctgtgcagcgccttcctctgcctcctcctccacgtggctctccgctcgctgctgcaccctccttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatgcttctgccgcctcttcctccgggcgccgcgggcagctcccgccggggccaccgggcctgccgatcctcggcgcgctgccactcgtgggcccagccccgcacgccggcctggccgcgctggcgcgcaagtacggtcccatcatgtacctgaagatgggcacggccggcgtggtggtggcgtcgtccccgcgcgcggcgcggacgttcctcaaggcgctggacgcgcggtacgccaaccggccggccgtggcgagcgccgcggacatcacgtacgggcggcagaacatggtgttcgcggactacgggcccaagtggaagctgatgcggaagctcgccagcgtgcacctgctcggcgcgcgcgcgctcgcggactgggcgtgcgtgcggcgcggcgaggccggccacgtgctgcgcggcgtggcggaggcggccgcggccggcaggcccgtcgtcgtgccggagctgctcgtgtgcgccctcgccaacatcgtcgggcagatcacagtgagcaagcgggtgttcgacgcgcagggggacgactcgaacagatacaaggacatgatcgtgtcgctgctgaccggcacgggcatgttcaacatcagcgacttcgtgccggcgctggcgcgtctggacctgcagggcgtgcaggcgaagctgcggcgcgtccaccgccagttcgacggcctcatcaccaagctgctggccgagcacgccgcgacggccgcggaccgcgcgcgccagggccgcccggacttcgtcgaccggctccgcgccacgatggacgccggcgccgccgccgacgacgagagcggcgagaccatcaccgaggtcaacatcaagggcctcatcttcgacatgttcacggcgggtacggacacgtcgtcgatcatcgtggagtgggcgatggcggagatgctcaagaacccgaccgtcatggcgcgcgcgcaggaggagctggaccgcgcggtgggccggggccggcgcctggaggagtcggacctgcccggcctcccctacctgcaggcggtgtgcaaggaggccatgcggctgcacccgtccacgccgctcagcctcccgcacttctccttggacgcctgcgacgacgtcgacggctaccgcgtcccggccaacacccgcctgctcgtcaacgtctgggccatcggccgggacccggaggcctgggagaggcccctcgacttccgccccgagcgcttcctgcccgggggcggcgcggagaaggtcgaccccctggggaactgcttcgagctcatcccgttcggcgccggccggaggatctgcgcggggaagctggcgggcatggtgttcgtgcagtacttcctgggcacgctgctgcacgcgttcgactggcgcctgcctgacggcgaggagaagctggacatgagcgagacgttcggcctcgcgctgcccaaggcagtgccgctccgcgccgtcgccacgccacggctcgtgccggaagcctatgcc

SEQ ID NO: 12 - protein, 66 aa, synthetic construct
  REGION 1..66: note = amino acid sequence encoded by SEQ ID NO: 11
MQLAALCTDPVVLCSAFLCLLLHVALRSLLHPPSAQKRAQTFEIGYSKIQKIKEFSVLTLFYAIHY

SEQ ID NO: 13 - DNA, 121 bp, synthetic construct
  misc_feature 1..121: note = marker SYN38529@61
  source 1..121: mol_type = other DNA
cgagttctggttcgagaagacgtcccagatcccgtcgctgccaattatcaagaactcgtcrtccttcgtcagcgtggtcatcttgagctctgggtcagcactcaggggaccacctgtctca

SEQ ID NO: 14 - DNA, 121 bp, synthetic construct
  misc_feature 1..121: note = marker SYN5866@61
  source 1..121: mol_type = other DNA
gacactaaaaaaacataatcctcgccggagcagccgagaaacgcgtggatctaggcggacmcagcatcggccccagatctgaggaacccccggcgggcggcctcaccttcggagatggcgt

SEQ ID NO: 15 - DNA, 121 bp, synthetic construct
  misc_feature 1..121: note = marker SYN11764@61
  misc_difference 64: note = n is a, c, g, or t
  source 1..121: mol_type = other DNA
ttgtgttttttcttttccttttacacagataataagcaatgggatcttcaagagcccagtrcancgggttgtaacaaacgcggagaaagagaggctatcagtggttctgttctattctata

SEQ ID NO: 16 - DNA, 101 bp, synthetic construct
  misc_feature 1..101: note = marker PZE-109015136@51
  source 1..101: mol_type = other DNA
cactattctttcctacatgatccagcaacaacctctttgcaaacttgcacrcgttagtaatgcattcattgacttgttcataagcacgctgctctctaagt

SEQ ID NO: 17 - DNA, 101 bp, synthetic construct
  misc_feature 1..101: note = marker PZE-109015397@51
  source 1..101: mol_type = other DNA
cttcttcgcaaggcacgggatggtggtgttgttgcccagcttcttcttcarctggcagaggcacctgtcgcttaggctgcagcagtccacatgcaccggca

SEQ ID NO: 18 - DNA, 101 bp, synthetic construct
  misc_feature 1..101: note = marker PZE-109016177@51
  source 1..101: mol_type = other DNA
attgtggacgacagatcagaaccttctgtgtcagagctatctccattctcragttgatagttgagtgcccatgtggtcagtggcctgcgggcatgtgctca

SEQ ID NO: 19 - DNA, 101 bp, synthetic construct
  misc_feature 1..101: note = marker PZE-109017276@51
  source 1..101: mol_type = other DNA
caactagtgttccatttgcagcagagtgccacttctgctcgtatgatcttmtacagacgagcagatgattacgggaacgtcaacccgctctccaggagcag

SEQ ID NO: 20 - DNA, 101 bp, synthetic construct
  misc_feature 1..101: note = marker PZE-109017407@51
  source 1..101: mol_type = other DNA
aagcctttttgtttgaggatctctaggatgaccgggtgttcaattatatcraatgtgttttgttttattcgaaaaaataccaaggacataaatcttaaatt

SEQ ID NO: 21 - DNA, 101 bp, synthetic construct
  misc_feature 1..101: note = marker PZE-109018614@51
  source 1..101: mol_type = other DNA
tgtaacaatctacgtggttctgtaaaaacaaattaaaaaatgtcatttttrtggtctattttcccagtgctgccataacatagtgatgatggtcacgtcca

SEQ ID NO: 22 - DNA, 121 bp, synthetic construct
  misc_feature 1..121: note = marker SYN32168@61
  misc_difference 88: note = n is a, c, g, or t
  misc_difference 119: note = n is a, c, g, or t
  source 1..121: mol_type = other DNA
gagccgatcggagatctctgcgagaacctcctcttgaacgactgtgacatgtgcgacatcrcagcagcggtctgctgctgtccattgncatcagatggcttgagtggggacgaagattnag

SEQ ID NO: 23 - DNA, 101 bp, synthetic construct
  misc_feature 1..101: note = marker PZE-109021152@51
  source 1..101: mol_type = other DNA
ccaacaataaagaggctttccttccttggtcttaccatgtgctgcaccacraaccaccacatcctcctgttcactattcttccctggcagcaaagtttcct

SEQ ID NO: 24 - DNA, 101 bp, synthetic construct
  misc_feature 1..101: note = marker PZE-109021389@51
  source 1..101: mol_type = other DNA
actgaaaaatctgatttagttgtaagctttggagaaattgttgtgagctcrttcatgactaggatctgggagacgtcacccaacaggactcgaacgacttc

SEQ ID NO: 25 - DNA, 200 bp, synthetic construct
  misc_feature 1..200: note = marker zm06133s02@101
  source 1..200: mol_type = other DNA
aggcgctcagaccggcaaggrgctcccgtcttgatttggccctgaaacggagtcatggaaaccaggtcagtacgcaagrcgttcgattcgacagtatcttsaccagttgacagcgtaccgtagacaggccaactgagaggtcagcaatgaaggtgtcctctgtctcgccactgaacaaatataataaagagaagattgra

SEQ ID NO: 26 - DNA, 101 bp, synthetic construct
  misc_feature 1..101: note = marker PZE-109022641@51
  source 1..101: mol_type = other DNA
gacataaattatcaacaagacaactgtggtgtcaccgaaaaaataattaarcaatcaatgcagcagtacttgtcctgcctctcgatcaggactacactaaa

SEQ ID NO: 27 - DNA, 66 bp, synthetic construct
  misc_feature 1..66: note = marker Affx-90814890@31
  source 1..66: mol_type = other DNA
acagatgaatctgccacacgaacgtccaagsagccggacagctgcgagcagagcttcaccaaaagg

SEQ ID NO: 28 - DNA, 121 bp, synthetic construct
  misc_feature 1..121: note = marker SYN34181@61
  source 1..121: mol_type = other DNA
attcgtcgcgtgatagctgtacagtttgacatctggattcgagtcgagtggtggtagttgraggtctactgtatatagttggcttcagtatcatggccgccgttgatctgaagccgacgtt

SEQ ID NO: 29 - DNA, 121 bp, synthetic construct
  misc_feature 1..121: note = marker SYN29297@61
  source 1..121: mol_type = other DNA
ttactcagcatcttcgacttgaaggcgaatttgacttgttcaagatagctagagcgacacmaggttttgttggtgctgacttgaaggcattagtggataaagcagggaatcttgcaatgaa

SEQ ID NO: 30 - DNA, 101 bp, synthetic construct
  misc_feature 1..101: note = marker PZE-109026962@51
  source 1..101: mol_type = other DNA
tcgatcgtcaggccacgtactcaccctccaacgtagaccttggtgccaagmagcagcgagtccatgaccgtggtgatgatcagcgacagcgagttgaacat

SEQ ID NO: 31 - DNA, 121 bp, synthetic construct
  misc_feature 1..121: note = marker SYN13048@61
  source 1..121: mol_type = other DNA
cttccatctgcaagtgtccgcacaaacaaaaactatttgtattttgtagaatctttggggmcggataggtgtgtggggcacataacatcttacttcggtcgctgtttgtgctgctagataa

SEQ ID NO: 32 - DNA, 101 bp, synthetic construct
  misc_feature 1..101: note = marker PZE-109063861@51
  source 1..101: mol_type = other DNA
ttcgaatctgatccatgattcagactaatttatgtttgatccatagtgacmagtttaatttcacagaaaaatacagcttagaatatgactccctgttgagt

SEQ ID NO: 33 - DNA, 101 bp, synthetic construct
  misc_feature 1..101: note = marker PZE-109029427@51
  source 1..101: mol_type = other DNA
ccatcagagcttccgttacagccagctagaaactgacgccatcagtgacgrccgttactagctagcagccattacaactcatccgttactagctataacac

SEQ ID NO: 34 - DNA, 200 bp, synthetic construct
  misc_feature 1..200: note = marker zm08444s04@101
  source 1..200: mol_type = other DNA
tgcagttagttgattatgcaaaactttgtctcagctaatggcggcgwtgttgctgctgccgaactcgatgatgtatgggaggatgtttcaggttctcctcrgcacgcggataccttagacagagaatggatccacaggcagagtcagtttcagaaggtattaattatcctagagtttatcyggaacrcacacagttgcta

SEQ ID NO: 35 - DNA, 200 bp, synthetic construct
  misc_feature 1..200: note = marker zm08444s03@101
  source 1..200: mol_type = other DNA
ctgactctgcctgtggatccattctctgtctaaggtatccgcgtgcygaggagaacctgaaacatcctcccatacatcatcgagttcggcagcagcaacawcgccgccattagctgagacaaagttttgcataatcaactaactgcaacatgagataagatagcactagtgcaagcagtactactaattaagcacaagac

SEQ ID NO: 36 - DNA, 101 bp, synthetic construct
  misc_feature 1..101: note = marker PZE-109031125@51
  source 1..101: mol_type = other DNA
cattaccataaggtgaagacaggcagatagcagcaacccttctacaccagrtagctgttacttatgagactggacaagcttgcttgttgattgttagatcc

SEQ ID NO: 37 - DNA, 101 bp, synthetic construct
  misc_feature 1..101: note = marker PZE-109033123@51
  source 1..101: mol_type = other DNA
atgcccaaagccgtagaatctactcggaggagagagatggagggagaaacraattcggtacacataccagggcaagccgagctccccgtaggaacccaaag

SEQ ID NO: 38 - DNA, 121 bp, synthetic construct
  misc_feature 1..121: note = marker SYN18776@61
  source 1..121: mol_type = other DNA
ggtggcacgggatcgtccgtccacatataagctgatgaaggagcagtcatgccgactgcaragtctatatggaaaagagggagaccctacttggaaatgctcaccctaagggctagtttgg

SEQ ID NO: 39 - DNA, 101 bp, synthetic construct
  misc_feature 1..101: note = marker PZE-109035519@51
  source 1..101: mol_type = other DNA
aaccagtcgcctaaacttgtgcattcatgtctgtgttgctccttctacccrttgctgcatgcagccgttggtcgtgtggagcaccactatagtccatatga

SEQ ID NO: 40 - DNA, 101 bp, synthetic construct
  misc_feature 1..101: note = marker PZE-109035852@51
  source 1..101: mol_type = other DNA
gcgagtgctgcaccatgccaggccaccatgatcagagcaagccagcaccamcaaagcaagcagaccttctatcaggtgatgttcatgtcagagcttggttc

SEQ ID NO: 41 - DNA, 101 bp, synthetic construct
  misc_feature 1..101: note = marker PZE-109036977@51
  source 1..101: mol_type = other DNA
tcgaaggcgccgcggaccataggaactccgtggccggagctctgcaattcrtgctttcagtaagaacatttcctctccttgtggtcgctctacgttctagg

SEQ ID NO: 42 - DNA, 200 bp, synthetic construct
  misc_feature 1..200: note = marker zm10851s01@101
  misc_difference 1..28: note = n is a, c, g, or t
  source 1..200: mol_type = other DNA
nnnnnnnnnnnnnnnnnnnnnnnnnnnnagcaaggaccaagttgctgagacaaaggtttgcatgtccaacatatttgtttcattgttatgggattaaaccmgcatacatgacaatgtgtctgttattggtatccatatccaatagcacaaggcagacattcattcttattcatcatcaaatggtcatttctaactcgtgg

SEQ ID NO: 43 - DNA, 101 bp, synthetic construct
  misc_feature 1..101: note = marker PZE-109041138@51
  source 1..101: mol_type = other DNA
attgcatatttgcttatgctttggatattggatttgaatatggatatactmctgtgatgtggtatgctgcttgtgcttggctaattggataaggtataaac

SEQ ID NO: 44 - DNA, 101 bp, synthetic construct
  misc_feature 1..101: note = marker PZE-109040213@51
  source 1..101: mol_type = other DNA
aggtatatcagatctgggtcaataaaatctagaccatcggttttagatccratggttattgacgcataccggttcatccgcggcaggatctaatcctggtc

SEQ ID NO: 45 - DNA, 101 bp, synthetic construct
  misc_feature 1..101: note = marker PZE-109043990@51
  source 1..101: mol_type = other DNA
tctcgctgacgactactatactactaatatatatgagaggatcggtcgcamgtacacggaagctgctggaacgcgtgcgtatcaaactagaataccagttg

SEQ ID NO: 46 - DNA, 101 bp, synthetic construct
  misc_feature 1..101: note = marker PZE-109045856@51
  source 1..101: mol_type = other DNA
cttgtctttcgccgactccatctttaattctgatgagacggctggctgctrtgttcgtcgtcagcacgcagtgcttcctcccttggtgtgtgtgcgtgcgt

SEQ ID NO: 47 - DNA, 101 bp, synthetic construct
  misc_feature 1..101: note = marker PZE-109048885@51
  source 1..101: mol_type = other DNA
gacaacatcgcgcgtggagctggtttggccggcgtctgttggaggcgatcmaactccatgagctcaatctaacgagccaagctcatcgccgcattgagtgt

SEQ ID NO: 48 - DNA, 128 bp, synthetic construct
  misc_feature 1..128: note = marker SYN32492@101
  source 1..128: mol_type = other DNA
taccctttgcctcagtgtaygccagctcgtaccagagtgagctgcttgatgtgttgccaaacctgtacagggtcatcctcgatggctccatgtgccagtcrgtgagctccaggttcttctccagctca

SEQ ID NO: 49 - DNA, 121 bp, synthetic construct
  misc_feature 1..121: note = marker SYN32488@61
  misc_difference 52: note = n is a, c, g, or t
  source 1..121: mol_type = other DNA
gatgaaggcctcaagtatttttttaggggactggaacacggtgaggcaaacnaaaaaaaamtagtcaccacaatcaaggttacctgtggtgatacagaatacttcactttaatgaacgcaa

SEQ ID NO: 50 - DNA, 200 bp, synthetic construct
  misc_feature 1..200: note = marker zm11758s02@101
  misc_difference 1..46: note = n is a, c, g, or t
  source 1..200: mol_type = other DNA
nnnnnnnnnnnnnnnnnnnnnnnnnnnnnnnnnnnnnnnnnnnnnnacaatcacaagcaatgccaatagcgccgccakcaatagagctggcgccacccgascactggcactgattatctggaccgggaacaggcagccgcgcgtggaggcgttcttgtctgtgatcttggcaagcccttcgaagtcgcaggcgcgcacat

SEQ ID NO: 51 - DNA, 101 bp, synthetic construct
  misc_feature 1..101: note = marker PZE-109051621@51
  source 1..101: mol_type = other DNA
ccaatctcccattcagaaatttgtatgaattcaggattcctaacagctgcraaatggtggacatgttaaatcatagaattcccagcacaaccatcggacag

SEQ ID NO: 52 - DNA, 101 bp, synthetic construct
  misc_feature 1..101: note = marker PZE-109053851@51
  source 1..101: mol_type = other DNA
actcaatggtcggataagacatctcaacggttaagctaacatgtcagggtmtggtagtgcattagatcggcccggtgccaccttgcacttgacatctagtt

SEQ ID NO: 53 - DNA, 101 bp, synthetic construct
  misc_feature 1..101: note = marker PZE-109053885@51
  source 1..101: mol_type = other DNA
atggtagcagcagcttggtctcagaaaaggtgtgatagttggtttccattmttcagtatacatatgattttgactactccctcttttggttctgctggcac

SEQ ID NO: 54 - DNA, 101 bp, synthetic construct
  misc_feature 1..101: note = marker PZE-109053887@51
  source 1..101: mol_type = other DNA
tgtgatttctagcgagaaggacagagtagtttctccaagttctgcccacamtgatgttgcaaactctcatcatcaatttatgcagcagtcgcagcagcagt

SEQ ID NO: 55 - DNA, 101 bp, synthetic construct
  misc_feature 1..101: note = marker PZE-109053906@51
  source 1..101: mol_type = other DNA
cggagtagtgtgagtggaggaaccgtggaagtggtctttgaaacaaggatmgcagatcaaagctgaaactgctttagtggagtactaggatgctaaagcca

SEQ ID NO: 56 - DNA, 101 bp, synthetic construct
  misc_feature 1..101: note = marker PZE-109054050@51
  source 1..101: mol_type = other DNA
agaccatgttcaagggtaaacttgacatatatgagattcctctttgcagcrctccaatgatgagtggtaggtgcatgcaaaaactggcaccttattgattg

SEQ ID NO: 57 - DNA, 101 bp, synthetic construct
  misc_feature 1..101: note = marker PZE-109054055@51
  source 1..101: mol_type = other DNA
tagaaactgctaacgatagaatgatcctgatggtagttgccttaaccgtargactgaaggtttcctcataatctctgtcatatcgctgcttaaagcctttg

SEQ ID NO: 58 - DNA, 101 bp, synthetic construct
  misc_feature 1..101: note = marker PZE-109054205@51
  source 1..101: mol_type = other DNA
tggtagagttagaaatacaaacgggaagagcttcactgatctatggagatmacagaacgaggatcacacggacatgcaactccctggggcaccctggccac

SEQ ID NO: 59 - DNA, 101 bp, synthetic construct
  misc_feature 1..101: note = marker PZE-109054458@51
  source 1..101: mol_type = other DNA
tacctgactaatccatctaattacaaggttgtcggctttgcgttgggagcrccgggtcaggggacgggcaaaggatcagatcgacagccgcacgagcattg

SEQ ID NO: 60 - DNA, 101 bp, synthetic construct
  misc_feature 1..101: note = marker PZE-109054723@51
  source 1..101: mol_type = other DNA
ctcagctgcagcaaaatccaggtcgaaaacgcctgcccacgatcgacaacmcccaatacactaaaacacatctacgtccccagaacggactgaaccagcat

SEQ ID NO: 61 - DNA, 101 bp, synthetic construct
  misc_feature 1..101: note = marker PZE-109054729@51
  source 1..101: mol_type = other DNA
tcttcttttatgttcttcaagagcagccaaagttacacggtacaactctartgaactttgagtttgagcaattgagttattttggaaataagtgctacttt

SEQ ID NO: 62 - DNA, 101 bp, synthetic construct
  misc_feature 1..101: note = marker PZE-109054805@51
  source 1..101: mol_type = other DNA
ttgtttttttttttttttttggcaacctggatcatgttagcactacacttmaacgaactctgctctactatgcagtactactagaagtcaaggaaaactcc

SEQ ID NO: 63 - DNA, 101 bp, synthetic construct
  misc_feature 1..101: note = marker PZE-109054812@51
  source 1..101: mol_type = other DNA
ccctggtacgcttgctgattgatgggctccagaggccggggatcttatccmtgactcaatgcagctaaccatagcctcatttgcgccatacctcgtgcatg

SEQ ID NO: 64 - DNA, 101 bp, synthetic construct
  misc_feature 1..101: note = marker PZE-109054943@51
  source 1..101: mol_type = other DNA
tgtgaaattttgacactgctctacggaataatgtgttgggatttgtcatgrtgaaattggctctgcaggaatggcaagctccgactcaaatcccaccgatg

SEQ ID NO: 65 - DNA, 121 bp, synthetic construct
  misc_feature 1..121: note = marker SYN32299@61
  source 1..121: mol_type = other DNA
tgaaggattcctctggtgctctgaataatggtctagggaggagcacttcatctcaagtaamcgaactgacacatcttcatagatcccacccgcctgctgactacaatccattctccttgca

SEQ ID NO: 66 - DNA, 101 bp, synthetic construct
  misc_feature 1..101: note = marker PZE-109058460@51
  source 1..101: mol_type = other DNA
tcatgcacagctatgaatttgatctcctatttggttgtgaatgtctggcartatgattaaagctgtagctgtacctaggctggaactctatgatttgcata

SEQ ID NO: 67 - DNA, 65 bp, synthetic construct
  misc_feature 1..65: note = marker Affx-91355778@36
  source 1..65: mol_type = other DNA
aatgctttgttgaacttttaatgcttctttgctggrctaggtagaagttctccttttagtgcttt

SEQ ID NO: 68 - DNA, 101 bp, synthetic construct
  misc_feature 1..101: note = marker PZE-109061769@51
  source 1..101: mol_type = other DNA
gatagagaacggaggagtagttcattcatgagaaacaccagtgcgtggatrttgacttgatgctgcacgtggcagtgccacgatgtcacgcttgcacgcga

SEQ ID NO: 69 - DNA, 121 bp, synthetic construct
  misc_feature 1..121: note = marker SYN36472@61
  source 1..121: mol_type = other DNA
gtattgtacgtccctcacagtcacatagaaagagagaggccgggatcgatatcgcgatagrtacgatggcggggatggccgcggccgcagtagggtcggcgtcgtcgtcgtccggcccggc

SEQ ID NO: 70 - DNA, 111 bp, synthetic construct
  misc_feature 1..111: note = marker PUT-163a-4646495-2099@56
  misc_difference 57..58: note = n is a, c, g, or t
  source 1..111: mol_type = other DNA
ctcgggactctagttttttcaacaaaaaaatagaacaaatatggcttcacaggggrnnaaaaaagggttcgcaagctcacaaacaaacctgtgaccttggataggaaagaa

SEQ ID NO: 71 - DNA, 101 bp, synthetic construct
  misc_feature 1..101: note = marker PZE-109065712@51
  source 1..101: mol_type = other DNA
ctcgtttttttgtcactcacacgtacgcgggcactgatctaacaacctgamcagagcttgaaggctacttgggtcgccgtgtgagcgaacgaagggtggag

SEQ ID NO: 72 - DNA, 71 bp, synthetic construct
  misc_feature 1..71: note = marker Affx-91059234@36
  source 1..71: mol_type = other DNA
gagatccctagttgggtattaattgattcggttcaycgtacttctcggatataaagattagatcttcgacc

SEQ ID NO: 73 - DNA, 101 bp, synthetic construct
  misc_feature 1..101: note = marker PZE-109066188@51
  source 1..101: mol_type = other DNA
agggcgaggaggcggagtagctgtgtgccagagcccagagggttggaatargtaaaagaaagctgctgccgcgtactacaagaacaacaacgcgggccgcg

SEQ ID NO: 74 - DNA, 101 bp, synthetic construct
  misc_feature 1..101: note = marker PZE-109067522@51
  source 1..101: mol_type = other DNA
ttatgccaatggactgtgcatttggcaggcgcgccagcgaaaaggacgaamcaccgcagcccatctaagattttggtataatcaggtggcaagaattagct

SEQ ID NO: 75 - DNA, 101 bp, synthetic construct
  misc_feature 1..101: note = marker PZE-109067146@51
  source 1..101: mol_type = other DNA
tttcaaaaacagagtagcagaacttctttttactagaatctgacagtgtargcttcttttcatcgtgttctttggtagggccgtgactcgtttgtaattaa

SEQ ID NO: 76 - DNA, 101 bp, synthetic construct
  misc_feature 1..101: note = marker PZE-109066949@51
  source 1..101: mol_type = other DNA
gaatctctctggtcgatctaggttcaaccggtttcaaacccgattcaatcrggattggctcagtcgagctaggtactcagccaagtactcgaccgagttct

SEQ ID NO: 77 - DNA, 101 bp, synthetic construct
  misc_feature 1..101: note = marker PZE-109066773@51
  source 1..101: mol_type = other DNA
gttgacatctgaaactgacaaagatgtggctaccttgttgtctgaaagagmtgtgttcacgactctttcgcaggaacaattggctgctgcgcgtaaccgta

SEQ ID NO: 78 - DNA, 101 bp, synthetic construct
  misc_feature 1..101: note = marker PZE-100001123@51
  source 1..101: mol_type = other DNA
acgtcaattccacgcaggtactagctcaccagtatcggttccatcttcagrcagatacctggcttgagcgcatcgatcggttgcatacaacactagaatga

SEQ ID NO: 79 - DNA, 101 bp, synthetic construct
  misc_feature 1..101: note = marker PZE-100000965@51
  source 1..101: mol_type = other DNA
cgtttcacgacaagtgcactcttatgtacgagtaataattcgcgtgttttraagcaatactgtacgatgaagtgttccgggagcctagaagtattttttag

SEQ ID NO: 80 - DNA, 121 bp, synthetic construct
  misc_feature 1..121: note = marker ZM011231-0277@61
  source 1..121: mol_type = other DNA
accgtcgtcgaacagcttgccctccacgatggccgcctcgaccttgtcgcggagctcggcragcgccttacgctcgctctccttgagatcgtccaggaagttgctctcctcccggaaagac

SEQ ID NO: 81 - DNA, 101 bp, synthetic construct
  misc_feature 1..101: note = marker PZE-109067561@51
  source 1..101: mol_type = other DNA
ccttgactgatcatgcatgcatggtaccatatatatgctgcgtgccttgtrtgacaaacagtgcaacgccacgtacggctgttgaagaaaaccaatccacc

SEQ ID NO: 82 - DNA, 101 bp, synthetic construct
  misc_feature 1..101: note = marker PZE-109067890@51
  source 1..101: mol_type = other DNA
aatttcaatagagattatgatggcagctagccatgcaacaaaagcacacaraagtaaagccatggagaagtacccattgtagctatccatactaactaacc

SEQ ID NO: 83 - DNA, 101 bp, synthetic construct
  misc_feature 1..101: note = marker PZE-109068071@51
  source 1..101: mol_type = other DNA
aacacacaaggatttagaatggttcgggccaccggagcgtaataccctacrttcactataaatcgtattgcttgagtttgagttcgagaggacctatcccc

SEQ ID NO: 84 - DNA, 121 bp, synthetic construct
  misc_feature 1..121: note = marker SYN21877@61
  misc_difference 42: note = n is a, c, g, or t
  source 1..121: mol_type = other DNA
tgagatcaacaaatcatgggcaaaatcacattatctacaacngaaaccatcctgacagggmaaaacatccaggagccttctcctatattccatcactctactttgagctatatattatatt

SEQ ID NO: 85 - DNA, 101 bp, synthetic construct
  misc_feature 1..101: note = marker PZE-109071755@51
  source 1..101: mol_type = other DNA
tgtactgcagctcgtacgtaccacatgcgaagccctgccgaagtgccgatrattggtaagattacaatcttggggtttcgttctcaatggtgcggaggcca

SEQ ID NO: 86 - DNA, 101 bp, synthetic construct
  misc_feature 1..101: note = marker PZE0008250961@51
  source 1..101: mol_type = other DNA
ttaaatagggaggcatgtccgtaagtgaatatagggacaaattcatccggmtatcttgatatgctctaggggaggtygatgatgatgagaaaaagccggag

SEQ ID NO: 87 - DNA, 121 bp, synthetic construct
  misc_feature 1..121: note = marker SYN29013@61
  source 1..121: mol_type = other DNA
ttgatgtatccggggctttcttgaataaatcgattgaatggaataatgaggcttatcgagmttaaacatgcctaaacggacggcacgccgcgagcacatgtaattaggaacttgcgtccta

SEQ ID NO: 88 - DNA, 101 bp, synthetic construct
  misc_feature 1..101: note = marker PZE-109072798@51
  source 1..101: mol_type = other DNA
aacaggaaggtaaaaaaaacgtacatatattgtggtgaagtctggaacatwcaaaaagattgtggtggacggatccggagggaggtggcggctaccaaacc

SEQ ID NO: 89 - DNA, 200 bp, synthetic construct
  misc_feature 1..200: note = marker SYN23066@101
  misc_difference 177..200: note = n is a, c, g, or t
  source 1..200: mol_type = other DNA
agtcagaattcagtgacgagatatgtgccgagaatggtgctgaasaggacgatgaaacagaggatttaatttggagcaaagaagagatcgatgctatttcrgcgctcttcgaccggccgatgcgccagaagcccctgaagccgcgaaaccccgcgaggcagagggcgcttccattgnnnnnnnnnnnnnnnnnnnnnnnn

SEQ ID NO: 90 - DNA, 101 bp, synthetic construct
  misc_feature 1..101: note = marker PZE-109073024@51
  source 1..101: mol_type = other DNA
tgctctgtcagcttgtcaagcctgcacttataaaagctccctgtgcctttrgtggcaagtcctcttcttcccctctaccggccggccgtttttcacttgac

SEQ ID NO: 91 - DNA, 101 bp, synthetic construct
  misc_feature 1..101: note = marker PZE-109073894@51
  source 1..101: mol_type = other DNA
gagtcttagcaattacagttctcaaaattttaaattgcaatttttcagaamcctcggcagccaactttttgttttcatgtaacaaaattaggtccctaaca

SEQ ID NO: 92 - DNA, 121 bp, synthetic construct
  misc_feature 1..121: note = marker SYN30845@61
  source 1..121: mol_type = other DNA
cagcacatattgtgttgaaatgaatgagcacactgcatccatatcaccactggtacacaarggcatagtttaagtcatgttatgatgtagcgcaccgagcaaggtattttgacacacataa

SEQ ID NO: 93 - DNA, 200 bp, synthetic construct
  misc_feature 1..200: note = marker ma60054s01@101
  source 1..200: mol_type = other DNA
atccggatccggggccaatgcacaacgtccatctttctaattctaaccagtttttctaaccagtattgttgagattacatgcgagaaagacgaagatgtcyatagagttcacaatttagaaagtgatagrtttctactrttacaacagttyaatcgattatatgtttatgttgatgatttttactaaatcrttttttaaa

SEQ ID NO: 94 - DNA, 101 bp, synthetic construct
  misc_feature 1..101: note = marker PZE-109074314@51
  source 1..101: mol_type = other DNA
aaggaatatactaccggccaagccgaggtccggttgccacttgaagcccaraaacgaaggtgcaacttaaaaaaagtacacgtgtctcgtgagcatcctat

SEQ ID NO: 95 - DNA, 121 bp, synthetic construct
  misc_feature 1..121: note = marker SYN22542@61
  source 1..121: mol_type = other DNA
aggccagcgagcaaatcgaatccgttggacttggacataacgcaacgcccgccgcgaaaarcactaccggcctgtgctgctgtggtagttagtttcaagcagcaatggaagccgtggtggc

SEQ ID NO: 96 - DNA, 101 bp, synthetic construct
  misc_feature 1..101: note = marker PZE-109074671@51
  source 1..101: mol_type = other DNA
ggtattctactgataagatatcggtccaggggttggcttagcaatgagctrgaccaggaggaaaaccggctaggtcgatccagaaaccggctgagcaagta

SEQ ID NO: 97 - DNA, 101 bp, synthetic construct
  misc_feature 1..101: note = marker PZE-109074676@51
  source 1..101: mol_type = other DNA
atctccttcgcccctccccacctggctcctgctctacacaagcagtcaagsgccataaagcgcagcaggatctccctctgcccaaaaccaaacctgcctcc

SEQ ID NO: 98 - DNA, 101 bp, synthetic construct
  misc_feature 1..101: note = marker PZE0004090171@51
  source 1..101: mol_type = other DNA
tgtcctagaagagaaggacaaagacgtatttgaaacgaaatggaatgttarttagtacccgggaccgggaggaaaaaaacaattatgctctcgcaaccata

SEQ ID NO: 99 - DNA, 101 bp, synthetic construct
  misc_feature 1..101: note = marker PZE-100001246@51
  source 1..101: mol_type = other DNA
ggcgtcccctggcttccgcgcctcctggatgatcgccatcacctcctccargctttgccccggggtgttcatgtctgccaactgcatgctctgctggtact

SEQ ID NO: 100 - DNA, 101 bp, synthetic construct
  misc_feature 1..101: note = marker PZE-100001337@51
  source 1..101: mol_type = other DNA
tcctcctatgatttttctacggcaccgcccatgagccctttaaaagtacarccgagcggctctcccagcgttctgctcccgctcggtccctgctctgcagc

SEQ ID NO: 101 - DNA, 101 bp, synthetic construct
  misc_feature 1..101: note = marker PZE-100001338@51
  source 1..101: mol_type = other DNA
caggagcagcgagcggcagagaaagtgcgacgagcagaaagaacatgaggragagagagtgtggttggaagaagataagcccaggcactgctgccgcgtgg

SEQ ID NO: 102 - DNA, 121 bp, synthetic construct
  misc_feature 1..121: note = marker SYN4635@61
  source 1..121: mol_type = other DNA
tgctcaatcttgcttcggggcaaatctcacctgctgtaagttgtcggttgagggtaatgcrcaaattgaacctcagaaggaggaactgtagacttattacatggttgttccacaacttcga

SEQ ID NO: 103 - DNA, 101 bp, synthetic construct
  misc_feature 1..101: note = marker PZE-109075140@51
  source 1..101: mol_type = other DNA
caattggcagcaaatttcaagcgtgaatgactggctacctgaccaatcatmcaacagtcagcaacattgccttgatagacagctagcctaggaggttggga

SEQ ID NO: 104 - DNA, 101 bp, synthetic construct
  misc_feature 1..101: note = marker PZE-109075353@51
  source 1..101: mol_type = other DNA
tatttaagagaaatgcagaatcttctttccggcacaaatccaacacaagcmtcttcctgagaacaagggtaaaccacaactctgtaccaagcaatcatcca

SEQ ID NO: 105 - DNA, 101 bp, synthetic construct
  misc_feature 1..101: note = marker PZE-109075487@51
  source 1..101: mol_type = other DNA
tgtactgtacgcacgcggagggaggcagtattgcagctgccgcaattcgcmgtgggtcggtggcagaagatccaaggccggaacaaaaataaacaataata

SEQ ID NO: 106 - DNA, 101 bp, synthetic construct
  misc_feature 1..101: note = marker PZE-109075564@51
  source 1..101: mol_type = other DNA
aacacatatgttcttcttcgcccctttcgttctttccacaaacatggaagmcgcagagccggggtttgcggccacgcgattttaatgttccgttgtcactt

SEQ ID NO: 107 - DNA, 101 bp, synthetic construct
  misc_feature 1..101: note = marker PZE-109075835@51
  source 1..101: mol_type = other DNA
atgtacgcacgcacgttttagcactccagcgctgaacctgcaatgcaagtrcaggtgcactgcaaatgagagagctggcagttacctctatatgaactggt

SEQ ID NO: 108 - DNA, 101 bp, synthetic construct
  misc_feature 1..101: note = marker PZE-109075980@51
  source 1..101: mol_type = other DNA
agcaaattggagggctagctctgaatagcttattagccaagtctgcataamcaatcaagccacccggccagtggaacggaagcacgaagcagcggcagcct

SEQ ID NO: 109 - DNA, 101 bp, synthetic construct
  misc_feature 1..101: note = marker PZE-109076467@51
  source 1..101: mol_type = other DNA
atttctacactctgttacaactacatagagggtccctagaagtctataaaraagtttccaaaaattgatatgcagtagtccggccgcacgcacttgaacaa

SEQ ID NO: 110 - DNA, 101 bp, synthetic construct
  misc_feature 1..101: note = marker PZE-109076558@51
  source 1..101: mol_type = other DNA
atcaactacaccactcacatggctactacagcggtggtggccttgaaggcraagtcacacaaagcgatcaaattcgagtgtcccatctgttttagagtgtt

SEQ ID NO: 111 - DNA, 101 bp, synthetic construct
  misc_feature 1..101: note = marker PZE-109076739@51
  source 1..101: mol_type = other DNA
ccaacctacataattcacatcaccattagcacacaccaatcgctgcaactrttttttagttgcttctggtgtctgtaagccatcgtatgagagtggcctac

SEQ ID NO: 112 - DNA, 101 bp, synthetic construct
  misc_feature 1..101: note = marker PZE-109076761@51
  source 1..101: mol_type = other DNA
ctactgctatatatcattggaagaagaagaaaagattaaacgtacgtttcrttttgtttctagatggcggctgaggtggcaaggatacgagcggccgggcc

SEQ ID NO: 113 - DNA, 101 bp, synthetic construct
  misc_feature 1..101: note = marker PZE-109077374@51
  source 1..101: mol_type = other DNA
ctctttctgcagctcgtgatcgagttgtgtgtgctccattcccaaattatrtatgatcgattagtagcgttaggggttaaccagtagccgctgtgtacttg

SEQ ID NO: 114 - DNA, 200 bp, synthetic construct
  misc_feature 1..200: note = marker ma61126d01@101; donor allele shows deletion of sequence asshown in SEQ ID NO: 115 between nucleotide positions 100 and 101.
  source 1..200: mol_type = other DNA
ttatttaataactaaattatataatacgaaaactaaaactatattttaatttctgtatttagcaatttaaaaactaaaataagataaaatgtagaaactacacacgttacgttcccttatccatcacaataggaaacagttattatatactgtgtttttataaataataaggcagaccttttcagcatggtgaccatgag

SEQ ID NO: 115 - DNA, 399 bp, synthetic construct
  misc_feature 1..399: note = deleted sequence in donor allele of ma61126d01 (SEQ ID NO: 114)
  source 1..399: mol_type = other DNA
aaatttagtacctaaaaccttagagcaactccaatagttatgtaaattttagctctctaaatcgcatatttaagaagttgctaaatgacttttggagtaaaaaaatgtgagttctccaatagttctctaaatataggttgtaattttgttttgtatttatccacataaaaaaataagtcacaaaaactatataatgcagtcaatatttttgtttagggagttgttaaatggttgccaaatgtagagagaaaatgaggttagatgacaagttgttaaatttagaaagttcatttagagaactgttggagaatagttttcatgttaactacctaaattattgatttaggaagtcttttaaagaactactggagttgctctaacaccctaaaagtcaaacta

SEQ ID NO: 116 - DNA, 101 bp, synthetic construct
  misc_feature 1..101: note = marker PZE-109077509@51
  source 1..101: mol_type = other DNA
atgtgggtgaagccaccagaggtgaacaaaaaacaaacaaacatgattcamtggccaccaagatatagctagttgcacagatagatcatgtttacacttct

SEQ ID NO: 117 - DNA, 101 bp, synthetic construct
  misc_feature 1..101: note = marker PZE-109077598@51
  source 1..101: mol_type = other DNA
catactgggaaccttgcagtcacgtccgtattgtccgttcaatcaatcgtrtgtacacagcacacttgacgacaccgatgcgtcaaaaaaatcgacacaca

SEQ ID NO: 118 - DNA, 101 bp, synthetic construct
  misc_feature 1..101: note = marker PZE-109077976@51
  source 1..101: mol_type = other DNA
gcaacggagctgagagagggagcttactgtggcagcagcagcactagctcrttgctctatctcctattcgcccaatagaatttcacaagagctggcaattt

SEQ ID NO: 119 - DNA, 101 bp, synthetic construct
  misc_feature 1..101: note = marker PZE-109078320@51
  source 1..101: mol_type = other DNA
ttaccgacggtaaaccgttaccgatgggcaacggtttttgtaggctaaacrgttttgtaaaccgtgtcctcaatgcaccataggtcttttcaactcgatta

SEQ ID NO: 120 - DNA, 200 bp, synthetic construct
  misc_feature 1..200: note = marker ma60405s01@101
  source 1..200: mol_type = other DNA
tcacaaacattttcaaagagtttttcacatcatcactaggcctaatgcatatgcaatgaattatgatatctagtgacacttaaataaccgattcaaccatrgagttctcttagggtgtgtttggttcagctttctgaactagattcgctgccaacaatctggaatcccaaccaaatggggtacaatagcactatagattc

SEQ ID NO: 121 - DNA, 101 bp, synthetic construct
  misc_feature 1..101: note = marker PZE-109078814@51
  source 1..101: mol_type = other DNA
tcagatcaaggactgattgatacgctgatggtcacagaaacatcgttgagrgcagcatcgctcggtcagtaactcagtagtgagtagccctgcaccttcag

SEQ ID NO: 122 - DNA, 101 bp, synthetic construct
  misc_feature 1..101: note = marker PZE-109078789@51
  source 1..101: mol_type = other DNA
catctctagttacggtggactagtgggaggggtagtagcagagtagtgctmtgtagctttgctttgcttgcttcgaagggaagcaattactcaaacccttc

SEQ ID NO: 123 - DNA, 101 bp, synthetic construct
  misc_feature 1..101: note = marker PZE-109079170@51
  source 1..101: mol_type = other DNA
tgacattgttcctgctgtcggtctcgagttcagagcatgtcggcagtgacrgcggaagctacggaacagatggcacagcgatggtgcgagcagctaatact

SEQ ID NO: 124 - DNA, 207 bp, synthetic construct
  misc_feature 1..207: note = marker ma61134d16@101; donor allele shows insertion of sequencegcggtct from nucleotide positions 101 to 107; wildtype alleledoes not show this insertion.
  source 1..207: mol_type = other DNA
ctcgcggcgttgtgcaccgaccccgtggtgctgtgcagcgccttcctctgcctcctcctccacgtggctctccgctcgctgctgcaccctccttctgcsgtctgccgcctcttcctccgggcgccgcgggcagctcccgccggggccaccgggcctgccgatcctcggcgcgctgccactcgtgggcccagccccgcacgccggcct

SEQ ID NO: 125 - DNA, 208 bp, synthetic construct
  misc_feature 1..208: note = marker ma61134d15@101; donor allele shows insertion of sequencegcggttct from nucleotide positions 101 to 108; wildtype alleledoes not show this insertion.
  source 1..208: mol_type = other DNA
ctcgcggcgttgtgcaccgaccccgtggtgctgtgcagcgccttcctctgcctcctcctccacgtggctctccgctcgctgctgcaccctccttctgcsgttctgccgcctcttcctccgggcgccgcgggcagctcccgccggggccaccgggcctgccgatcctcggcgcgctgccactcgtgggcccagccccgcacgccggcct

SEQ ID NO: 126 - DNA, 200 bp, synthetic construct
  misc_feature 1..200: note = marker Affx-91110408@101
  source 1..200: mol_type = other DNA
cgaaagattgtcacatttaatttcattaagcaaacttgcactctaacaaaatgtttcactggcaatataactatacaggtagttccattgtaattgtgcaraaaaggaacaacctcgcaatgttttcaaacaaatgttctctgggattggccgattagatgattttcaggcakttcatacatagtgccaaaggtgcatca

SEQ ID NO: 127 - DNA, 111 bp, synthetic construct
  misc_feature 1..111: note = marker PUT-163a-16926216-1143@56
  source 1..111: mol_type = other DNA
gcaagcctggtttgtttgggtcatcaaggatcaaatggaactttaccaagtttctsgtggacaaagatgggaaggtcatcgagagatacggaacctcgacagctccaatgg

SEQ ID NO: 128 - DNA, 71 bp, synthetic construct
  misc_feature 1..71: note = marker Affx-91121762@36
  source 1..71: mol_type = other DNA
atctctgtctgcggcttatcagtcgtgcgtccaagrgatgaacctttcaggtcgaatcgcctatggataga

SEQ ID NO: 129 - DNA, 101 bp, synthetic construct
  misc_feature 1..101: note = marker PZE-109080508@51
  source 1..101: mol_type = other DNA
catgctcaacctgcagctcggcatcaggtaggtacccaccttaataaccgrccgtatctcgtttcttcatttcagacatttttccggtaattgatggccac

SEQ ID NO: 130 - DNA, 101 bp, synthetic construct
  misc_feature 1..101: note = marker PZE-109080569@51
  source 1..101: mol_type = other DNA
agctaattcggatacaccgtatctgtaaaatacggcgcagctacatatatrtggcgacctaattaagcacggcaccagccgtaataataatttatatagac

SEQ ID NO: 131 - DNA, 200 bp, synthetic construct
  misc_feature 1..200: note = marker ma60182s01@101
  source 1..200: mol_type = other DNA
ccccatcccattccgagaattgagaacataacgtatactcgtaaagcgtaaaataacgaacgatttcatccggtgaggcttcgtggaaaggggaggaagargggagcgaacgtacatgatagcgacgcatccggccgcagcggcgagggcsgcgaggatggcgagcttgaagacggagaggaggaggccgtggcggagga

SEQ ID NO: 132 - DNA, 101 bp, synthetic construct
  misc_feature 1..101: note = marker PZE-109080822@51
  source 1..101: mol_type = other DNA
cgacgcactacatatttgtggtactgtggttcaagggatttgacgaattcrtgaccgggatcttcgtttgacagggatttcgtgactgggttccacaagcg

SEQ ID NO: 133 - DNA, 200 bp, synthetic construct
  misc_feature 1..200: note = marker ma61125s01@101
  source 1..200: mol_type = other DNA
ttatgggggaggcagattgtcgtggggcgcgggggagggcgagcttgggggcgccgcagagcgagggacattgccgagattgccatggagtgagggcacgrcgagattgccgcgagggagagggcgcttgccgaggcagattgccgcgggggaggggcgcttgccgaggcggagacgtggtgaagccgtggtggacgccc

SEQ ID NO: 134 - DNA, 121 bp, synthetic construct
  misc_feature 1..121: note = marker SYN29273@61
  source 1..121: mol_type = other DNA
taaggatcgatatttacaggtaaaaaataaggagacttcaacatggtgggaaaatactccrtggcggcacctgcacgctgttttgcaaacgaggcatgcaatgataaagtagtttgttggt

SEQ ID NO: 135 - DNA, 101 bp, synthetic construct
  misc_feature 1..101: note = marker PZE-109081405@51
  source 1..101: mol_type = other DNA
tatcgttacgggctggctggaaattcgaccagtgtttctccccattagcargcccaacccagcagccttaacttcttatcttagtgggttgaggttatata

SEQ ID NO: 136 - DNA, 101 bp, synthetic construct
  misc_feature 1..101: note = marker PZE-109081479@51
  source 1..101: mol_type = other DNA
tgaaacgtcgagctatgtataggtccacatcttcgggagcttctacttctrgctctcccaaaaacacagctaccactaaaagaattggcaagtcgatgcat

SEQ ID NO: 137 - DNA, 71 bp, synthetic construct
  misc_feature 1..71: note = marker Affx-90937015@36
  source 1..71: mol_type = other DNA
atacgatgtgattggtacacttccctaaggcttgayagtatatggatacaactttttcttatgttatgtca

SEQ ID NO: 138 - DNA, 101 bp, synthetic construct
  misc_feature 1..101: note = marker PZE-109081680@51
  source 1..101: mol_type = other DNA
catatgatggtatgggatcgaagagcattcgagtactcggcggcaggtgaraaagccatggacgctcacctgaacgacaggggccgccgcagctcctgacg

SEQ ID NO: 139 - DNA, 121 bp, synthetic construct
  misc_feature 1..121: note = marker SYN29834@61
  source 1..121: mol_type = other DNA
cctttccttccttgaagatcaaaaggaggacgtgtggcagcgtctgggaagaagggaaggragagtttcggtgtggcagcgtctctctatgaagaaggaagccctccccccggtgggtgct

SEQ ID NO: 140 - DNA, 101 bp, synthetic construct
  misc_feature 1..101: note = marker PZE-109082046@51
  source 1..101: mol_type = other DNA
aatgtgggaagagctggagcagcaaatctagacgtcattcaagaagtcgartatgtaaaggaagatgctagaattatatacctccttgaatgcctccaaaa

SEQ ID NO: 141 - DNA, 101 bp, synthetic construct
  misc_feature 1..101: note = marker PZE-109082004@51
  source 1..101: mol_type = other DNA
gcttggaagaggccaagacgtacgcgcgcaggccgcagcacacgacacacracatgacatccagctttaaattcattattgctagatcaactcaaagagtt

SEQ ID NO: 142 - DNA, 71 bp, synthetic construct
  misc_feature 1..71: note = marker Affx-90383113@36
  source 1..71: mol_type = other DNA
catgtatgtgcatcaggaggaggatccttggaactygaatctttcccgtgcggccattgatggcatccttt

SEQ ID NO: 143 - DNA, 121 bp, synthetic construct
  misc_feature 1..121: note = marker SYN34099@61
  misc_difference 60: note = n is a, c, g, or t
  source 1..121: mol_type = other DNA
ctcaccatctaaattcatggtttggatataagctataccatttggatcgctcgtacccanragaaccatgtcacacggcatggtctcattggcatgaattttgacaacctcccctgcacaa

SEQ ID NO: 144 - DNA, 45 bp, synthetic construct
  misc_feature 1..45: note = marker Affx-91186901@10
  source 1..45: mol_type = other DNA
acgagcactsaaagcgggattaatactccacaagcttctactgga

SEQ ID NO: 145 - DNA, 121 bp, synthetic construct
  misc_feature 1..121: note = marker SYN33659@61
  source 1..121: mol_type = other DNA
gcagacctgtgttaaccgttgaacccgtgagacggcgtcttcatgcaccacgactgtatcrgagactatatccagcacgtcttcgacaaataaacagtacgtgcttacctgcaaagggaag

SEQ ID NO: 146 - DNA, 101 bp, synthetic construct
  misc_feature 1..101: note = marker PZE-109082918@51
  source 1..101: mol_type = other DNA
cgacttgctcatcacaacgttaggcgcataactgatgcagattgcagagtracactgacacacgtcgtttgttattactcatgattccaagctggatatca

SEQ ID NO: 147 - DNA, 101 bp, synthetic construct
  misc_feature 1..101: note = marker PZE-109083369@51
  source 1..101: mol_type = other DNA
cacagaacagaacttggcatgcagtgcagcggccggcaacagctttgtgcrctctttggttctttaaagcggcgcgtcttttctttgcgcaaattgatgat

SEQ ID NO: 148 - DNA, 101 bp, synthetic construct
  misc_feature 1..101: note = marker PZE-109083328@51
  source 1..101: mol_type = other DNA
ttggaacatttcaaggagcccgcttaaatttgatctgcattctcatgggargtgggggcagcatcctagacctacctccttcctaaatctgtctttcgcac

SEQ ID NO: 149 - DNA, 121 bp, synthetic construct
  misc_feature 1..121: note = marker SYN29817@61
  source 1..121: mol_type = other DNA
acattaccagccaagagaggcctatgcttctccaagaacaatcacttgtacgctctacacratcaccaaatctgactcgtcttcctcggtcttggaaagagaacaagagcaagcgacaaaa

SEQ ID NO: 150 - DNA, 200 bp, synthetic construct
  misc_feature 1..200: note = marker ma60360s01@101
  source 1..200: mol_type = other DNA
gtcctgttgcatctgaatatataatctcacctcaactgctcaacastcaccaacctgtctgtccctgacggctgacgttcaaataatgcgcccaccctgtmtgtcrccaccacaccacacgtccacgtaggttctgggcaackcaattatyccgcgtggtygaccgatcagccagccggctggaagagaaagagaaccgt

SEQ ID NO: 151 - DNA, 200 bp, synthetic construct
  misc_feature 1..200: note = marker ma61158s01@101
  source 1..200: mol_type = other DNA
tggtgagttggtgactaaagtgggataactaaaatttagttttttagtcattaaaggggtgactaaaaaggactaaagtaggatttttaccttatttgccytcttcactttatttttcagtaaacatccactaattaatagaggtaatacagtcattattcccagcagttaatggtctttagtccggtttagtcattgga

SEQ ID NO: 152 - DNA, 200 bp, synthetic construct
  misc_feature 1..200: note = marker ma61036s01@101
  source 1..200: mol_type = other DNA
atgtagacataatgtatatctaagtgcctagcaaaatctatgcatctaaaaaaccaaagcatctgatctaataatttggaatgaagggagtagttcttaakaaacataatgcattgcatcatggtgtccagcaaaataattgcacaaatactgaagaatacttcacatacaagtatacaacacagttacaatcaggtgtt

SEQ ID NO: 153 - DNA, 101 bp, synthetic construct
  misc_feature 1..101: note = marker PZE-109086494@51
  source 1..101: mol_type = other DNA
gtttgaaaagagggcgcaagtgcacaatcagccaatcacgtttgtctcggmagtctgcagccaataatgtgacatgcaaataccctactatcaggctctct

SEQ ID NO: 154 - DNA, 200 bp, synthetic construct
  misc_feature 1..200: note = marker ma61161s01@101
  source 1..200: mol_type = other DNA
atcttctatattttttaaacttttaaaataaaatagtgtctatgctggcacgagcactattagctgttagtgccgtatagtgtcgatatgataggattagrcactataatagtgtctgggccgctagttagacactagtgctaatacgtcacgacatgattactaatagtgtctaatagtgtcgtactttaatagtgata

SEQ ID NO: 155 - DNA, 200 bp, synthetic construct
  misc_feature 1..200: note = marker ma60806s03@101
  source 1..200: mol_type = other DNA
cctattatyaagcatttacacgtactacaagctatctgacgacggcggtactatagtaaaaggggaaaaaaatctagggaaaaaaccaaaacagatcacarggcgatgtagaaagttggagctctaacagaatctagctatcacaattcacacgacaggagaggacaaattgaacaataataataatcgatcgatccctc

SEQ ID NO: 156 - DNA, 200 bp, synthetic construct
  misc_feature 1..200: note = marker ma61071s01@101
  source 1..200: mol_type = other DNA
tataaagcataactggtgtgcagtgcaggtgcctatttctcctaatgtacaacctatgttagctgggctttattttttaaaaaaaagttcagtataagtgyaacggccatgaaatcctttttaattggcattataggaattaatacaagatagaagaacatgaaaaattgaaaatygtgacttaatamttgagcttcagt

SEQ ID NO: 157 - DNA, 121 bp, synthetic construct
  misc_feature 1..121: note = marker SYN38267@61
  misc_difference 55: note = n is a, c, g, or t
  source 1..121: mol_type = other DNA
cgtaaccccatcaggaaccagctcttcacttttcatcttgtggaacaaaatgacnacatcrgatccaagaccatgcgatgccaagccactaatcatagtattccagacagaaatatggttt

SEQ ID NO: 158 - DNA, 101 bp, synthetic construct
  misc_feature 1..101: note = marker PZE-109088808@51
  source 1..101: mol_type = other DNA
ttagtctgcagctataaatgttgtgccgacacaggtatttcgctcagtcarcaaaagtccgacgtgtttttcgatttggtcgagattcctgctcagtgacg

SEQ ID NO: 159 - DNA, 110 bp, synthetic construct
  misc_feature 1..110: note = marker PUT-163a-60348412-2592@55
  source 1..110: mol_type = other DNA
caaacatgatgtacctagccatcccagataaacaggcgactgagaataagtctawcaaccaagaaagacatgtagatgactgacaactcagatttcacccaacacacaca

SEQ ID NO: 160 - DNA, 121 bp, synthetic construct
  misc_feature 1..121: note = marker PHM1599.84@61
  source 1..121: mol_type = other DNA
atctccggtrtcactgacatagkggtcttgbttyaagctytktgccagmacccggaatagrtgccacggcagagcacccgacttcttcggctctttgtagtacttgccaagaaccggcttt

SEQ ID NO: 161 - DNA, 101 bp, synthetic construct
  misc_feature 1..101: note = marker PZE-109090188@51
  source 1..101: mol_type = other DNA
aatacatgcttgaatagctggaaggtgggggttccgttgtggctgttgtaractgccaaaagtttgcgctgcacatggaaaataatgactaagaactaacg

SEQ ID NO: 162 - DNA, 200 bp, synthetic construct
  misc_feature 1..200: note = marker ma60984s01@101
  source 1..200: mol_type = other DNA
cgcttcaaagttttatgttgatatcttttgcacaatataatctgctatgacatttggccttatatcaacttttgtgtcaactaaatatgcctttgtcgttygtattatgttcaagtattcaactttataagagtttgatatacctcttgtggacatagaatcaaagaactaaaagatttagatgatacaacatatactga

SEQ ID NO: 163 - DNA, 101 bp, synthetic construct
  misc_feature 1..101: note = marker PZE-109091377@51
  source 1..101: mol_type = other DNA
tggacaaagacagtagtaacaaggaagctaaaaatccagcaactacgatgrtgaaaaccaaaacaaatacttaaactaagcaagtctctctctctcttttt

SEQ ID NO: 164 - DNA, 101 bp, synthetic construct
  misc_feature 1..101: note = marker PZE-109092033@51
  source 1..101: mol_type = other DNA
ttcttgacgatctcatcactgagcgaggcgatctgggcgtcgagcgccttracagcctcctccttctgcgcctcgaggttggcgagcgcctctacgagctc

SEQ ID NO: 165 - DNA, 101 bp, synthetic construct
  misc_feature 1..101: note = marker PZE-109092182@51
  source 1..101: mol_type = other DNA
attgcaacaaaactacactttgagacaaggccatgctcaggaatgtttttrtattacatcttaggaatctgcattttatttgcatgcaatattcttctatc

SEQ ID NO: 166 - DNA, 121 bp, synthetic construct
  misc_feature 1..121: note = marker SYN27192@61
  misc_difference 43: note = n is a, c, g, or t
  source 1..121: mol_type = other DNA
cattgctgataggccatgaccaattctatattggtgacagatnagatacagtttacaacarattacacttgccttccagaaattcctggaaactagacagagtgctacagcaccactaatc

SEQ ID NO: 167 - DNA, 101 bp, synthetic construct
  misc_feature 1..101: note = marker PZE-109093491@51
  source 1..101: mol_type = other DNA
aacggtaggaaattttcttcaaaaaatagcagttagtatactactagagcraaagctgcaaccaaacttgtaggatccatctggacatgtgttgtatccaa

SEQ ID NO: 168 - DNA, 101 bp, synthetic construct
  misc_feature 1..101: note = marker PZE-109093846@51
  source 1..101: mol_type = other DNA
gactgcaggagtcctagtcgttcttgccggagtttaagtagagacatcgaraaagaaacaaatggcacccccttcactaaaaacagagctgtgcactctag

SEQ ID NO: 169 - DNA, 101 bp, synthetic construct
  misc_feature 1..101: note = marker PZE-109094238@51
  source 1..101: mol_type = other DNA
aagacgaagacggcacaccatcggttgtgggcttgagcatactagccggaratctggatagacttgacgtcggccttcttggcctcctccttaggcacggt

SEQ ID NO: 170 - DNA, 101 bp, synthetic construct
  misc_feature 1..101: note = marker PZE-109094399@51
  source 1..101: mol_type = other DNA
accaaagaatttgtacattgtacccaaacagctcgcgagtttgtgtggctrgctgcattcaaatgtccacagtttgactgacgacggcttgtatgcaattc

SEQ ID NO: 171 - DNA, 80 bp, synthetic construct
  misc_feature 1..80: note = marker ZM013688-0470@20
  source 1..80: mol_type = other DNA
ccatgatgcagttttcccgrtgagggcactcaaacgttgccaagatcggtgaattcttatcggcggcatcagctggagga

SEQ ID NO: 172 - DNA, 101 bp, synthetic construct
  misc_feature 1..101: note = marker PZE-109096103@51
  source 1..101: mol_type = other DNA
gatggcggtggtatgggcggtgttctcttccaactctgagctccaggtgcraccacgggattccaggttcattcaccatttgctctgtaacactattggtg

SEQ ID NO: 173 - DNA, 121 bp, synthetic construct
  misc_feature 1..121: note = SYN22829@61
  source 1..121: mol_type = other DNA
ataatcacctgccagcgaccgtggcgtcaaaggtgaaggcgacgaggaagcggccagggcrttcttcatcaggctcgatccgaagcgtctccttcttaaggttgatgtcgttgcggatagt

SEQ ID NO: 174 - DNA, 121 bp, synthetic construct
  misc_feature 1..121: note = marker SYN22625@61
  source 1..121: mol_type = other DNA
caagtagattgcataccttgagatcttccaaatcttcatctttaatttttttaaccttggrtgtcttggcattacttccagcagcacctgcacttttcttctgcgccgttggctgaaaggt

SEQ ID NO: 175 - DNA, 101 bp, synthetic construct
  misc_feature 1..101: note = marker PZE-109097654@51
  source 1..101: mol_type = other DNA
cgcggatcgggccggacggtccggcgctcagtttggtgctcaactgaaacrctagtgtaacatagatgactttgtttttttagctgagctttctaacaaac

SEQ ID NO: 176 - DNA, 101 bp, synthetic construct
  misc_feature 1..101: note = marker PZE-109097752@51
  source 1..101: mol_type = other DNA
ggtgcaatcaaaggattctaagagaatatataatatggatgatgtcctgcragaagaaacttgccataaagcctactgattagctgctgataaataatact

SEQ ID NO: 177 - DNA, 101 bp, synthetic construct
  misc_feature 1..101: note = marker PZE-109098108@51
  source 1..101: mol_type = other DNA
aacagctcaaatctcaacacaaaactaatcgccttgaactaaaatccacarcacaaggagcagcgtttgggccttccttgtttgcaggagcaagctgcctt

SEQ ID NO: 178 - DNA, 200 bp, synthetic construct
  misc_feature 1..200: note = marker zm00728s04@101
  source 1..200: mol_type = other DNA
ctaccaagaagtactgcaaaagagtaccgaagatatgagtgtaragttctgtttgaggacgtcagtgggaagagtatcatgtaaggcagttcacctccacwgcaagtataattggtatgaacatcttccagcgattgttagggtttggaccatcaattaacttctgatcaacatgaatgggcttgctcccgtttgagata

SEQ ID NO: 179 - DNA, 101 bp, synthetic construct
  misc_feature 1..101: note = marker PZE-109098623@51
  source 1..101: mol_type = other DNA
gatttgagacagccattgacgcctgtgttggcgtggcagcatgagctgaaragtcctagtaatgttgctatgtttcgactttctgaattgaggccttccaa

SEQ ID NO: 180 - DNA, 121 bp, synthetic construct
  misc_feature 1..121: note = marker SYN27711@61
  source 1..121: mol_type = other DNA
taaagaataacattaagataagtgcatcctgaacgtgcaacgtgttggtaacaaatgatgraaggacgactgatatatgatgttgacgtctttcagtcgacagcaaaaattaaggccactc

SEQ ID NO: 181 - DNA, 121 bp, synthetic construct
  misc_feature 1..121: note = marker SYN27709@61
  source 1..121: mol_type = other DNA
atggagaggattcttccagccaaatgaaaacaagctcaagttcttttatgatgggcaaggrtcatgtccatccagctataccaggaccaagctgaattcaccttctacgatgctgatggaa

SEQ ID NO: 182 - DNA, 101 bp, synthetic construct
  misc_feature 1..101: note = marker PZE-109099670@51
  source 1..101: mol_type = other DNA
tctcgcttctcgtctacatctaagcatgcctacacctgcggtttccagatmgggtgtttggtacgatcgagcaacggtgaatctcgctctgttatttgttt

SEQ ID NO: 183 - DNA, 71 bp, synthetic construct
  misc_feature 1..71: note = marker Affx-91229917@36
  source 1..71: mol_type = other DNA
cattatgcacgccccggcccctcattagcacggagscgagagtaattagcgccagtcaagaggattcgtgt

SEQ ID NO: 184 - DNA, 101 bp, synthetic construct
  misc_feature 1..101: note = marker PZE-109100755@51
  source 1..101: mol_type = other DNA
gactgtctgattgaaagttcagattcaactgccatggctgcaggttcatgrtcgattggttgcgccgaaaaaagcagtggaccaaggcgaatgaaaaagtc

SEQ ID NO: 185 - DNA, 121 bp, synthetic construct
  misc_feature 1..121: note = marker SYN25124@61
  misc_difference 99: note = n is a, c, g, or t
  misc_difference 108: note = n is a, c, g, or t
  source 1..121: mol_type = other DNA
atgaaatgactgcgatatggtggcagtctatcatatagaaatctaaaacgggtattcatgrgtccatctccacgggttctgtggctgtaaaaattggtnccttgcccntaatcactgcagt

SEQ ID NO: 186 - DNA, 121 bp, synthetic construct
  misc_feature 1..121: note = marker SYN25123@61
  misc_difference 14: note = n is a, c, g, or t
  misc_difference 52: note = n is a, c, g, or t
  source 1..121: mol_type = other DNA
acgggtattcatgngtccatctccacgggttctgtggctgtaaaaattggtnccttgcccmtaatcactgcagtcatcatcaatttaccagcatctggaaaatcctcgtgctgcatcaaca

SEQ ID NO: 187 - DNA, 101 bp, synthetic construct
  misc_feature 1..101: note = marker PZE-109101695@51
  source 1..101: mol_type = other DNA
agaagataggaaacagttaacgcatgaactgtcttcacatataagagcacrcgttgcagaaagatctgctgccaaacagcaatacaaacaatcaaagaaag

SEQ ID NO: 188 - DNA, 101 bp, synthetic construct
  misc_feature 1..101: note = marker PZE-109101698@51
  source 1..101: mol_type = other DNA
ctggatttccttctggtgaccatatgttggggctgcaagttaagaataatrttaaaacaatttatcagatgacttgctgaataaaatgagctacgacacac

SEQ ID NO: 189 - DNA, 101 bp, synthetic construct
  misc_feature 1..101: note = marker PZE-109102054@51
  source 1..101: mol_type = other DNA
gacgagaagctcctcgtctacgactacatgtccatgggcagcctctccgcmctgctgcacggtaaaatttcccgccttccaccatttttaccgtttcttcc

SEQ ID NO: 190 - DNA, 101 bp, synthetic construct
  misc_feature 1..101: note = marker PZE-109102157@51
  source 1..101: mol_type = other DNA
agcaaactctatatagactggcaactcaataggagtagcagcagggtaaaraatttgaagaattttgaatgtctcggtggccgctcaacttatctaccata

SEQ ID NO: 191 - DNA, 200 bp, synthetic construct
  misc_feature 1..200: note = marker zm02908s02@101
  source 1..200: mol_type = other DNA
gaagatccaagctccccgaaggatcattyactcccgaagckatggctccttccagcgcagcacctgaaacagcagcttctagagcatctatgttcttgtargagacctccttcttgcacaaacccaggacaaggcttctgattctggggatcttcgaacaacctccgaccagtatgacatcattgatgtcctctggtacc

SEQ ID NO: 192 - DNA, 47 bp, synthetic construct
  misc_feature 1..47: note = marker Affx-90454668@36
  source 1..47: mol_type = other DNA
gctgctgtttcaggtgctgcgctggaaggagccatmgcttcgggagt

SEQ ID NO: 193 - DNA, 111 bp, synthetic construct
  misc_feature 1..111: note = marker PUT-163a-60357079-2822@56
  source 1..111: mol_type = other DNA
caattgataataagcctgccactgatgaactggatagcttgatcaggttaaagcargctgaagcatatatgtaccaggaacgtgcaaatgatgcacggaatgaggttgaca

SEQ ID NO: 194 - DNA, 71 bp, synthetic construct
  misc_feature 1..71: note = marker Affx-90988936@36
  source 1..71: mol_type = other DNA
aaatactcatgtatgaaagctacaaatatacatcarcaatctctctcagtatttctgtcaaaattggcagt

SEQ ID NO: 195 - DNA, 101 bp, synthetic construct
  misc_feature 1..101: note = marker PZE-109103504@51
  source 1..101: mol_type = other DNA
gcggagacaacgacggtgaggtgggcgggatagaggcaaactcactacaamatattgagatgatagggaggcgatagctccggtgagaaccgtctaaatag

SEQ ID NO: 196 - DNA, 387 bp, synthetic construct
  misc_feature 1..387: note = marker ma61134xxx; donor allele shows insertion of sequence ofSEQ ID NO: 197 from nucleotide positions 101 to 287; wildtypeallele does not show this insertion.
  source 1..387: mol_type = other DNA
accatgcagctcgcggcgttgtgcaccgaccccgtggtgctgtgcagcgccttcctctgcctcctcctccacgtggctctccgctcgctgctgcaccctccttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatgcttctgccgcctcttcctccgggcgccgcgggcagctcccgccggggccaccgggcctgccgatcctcggcgcgctgccactcgtgggcccagccccgca

SEQ ID NO: 197 - DNA, 187 bp, synthetic construct
  misc_feature 1..187: note = insertion in donor allele of ma61134xxx (SEQ ID NO: 196)
  source 1..187: mol_type = other DNA
cttctgcccagaagcgggcccagacatttgagattgggtattcaaaaattcaaaagattaaagaatttagtgttctaacgctattttatgcaatacattattgacaaattagtgttctaacactatagatcaccaaaaacatgggtattcaatgaatacccatgaaacccccctgggcccgcccatg

SEQ ID NO: 198 - DNA, 38 bp, synthetic construct
  misc_feature 1..38: note = ma61134d01 Primer_AlleleX
  source 1..38: mol_type = other DNA
gaaggtgaccaagttcatgctgcaccctccttctgccc

SEQ ID NO: 199 - DNA, 40 bp, synthetic construct
  misc_feature 1..40: note = ma61134d01 Primer_AlleleY
  source 1..40: mol_type = other DNA
gaaggtcggagtcaacggattctgcaccctccttctgccg

SEQ ID NO: 200 - DNA, 18 bp, synthetic construct
  misc_feature 1..18: note = ma61134d01 Primer_Common
  source 1..18: mol_type = other DNA
ccgcggcgcccggaggaa

SEQ ID NO: 201 - DNA, 41 bp, synthetic construct
  misc_feature 1..41: note = ma61134d02 Primer_AlleleX
  source 1..41: mol_type = other DNA
gaaggtgaccaagttcatgctcaaatgtctgggcccgcttc

SEQ ID NO: 202 - DNA, 39 bp, synthetic construct
  misc_feature 1..39: note = ma61134d02 Primer_AlleleY
  source 1..39: mol_type = other DNA
gaaggtcggagtcaacggattcgcccggaggaagaggcg

SEQ ID NO: 203 - DNA, 21 bp, synthetic construct
  misc_feature 1..21: note = ma61134d02 Primer_Common
  source 1..21: mol_type = other DNA
gctcgctgctgcaccctcctt

SEQ ID NO: 204 - DNA, 41 bp, synthetic construct; identical to SEQ ID NO: 201
  misc_feature 1..41: note = ma61134d04 Primer_AlleleX
  source 1..41: mol_type = other DNA

SEQ ID NO: 205 - DNA, 41 bp, synthetic construct
  misc_feature 1..41: note = ma61134d04 Primer_AlleleY
  source 1..41: mol_type = other DNA
gaaggtcggagtcaacggattcaaatgtctgggcccgcttg

SEQ ID NO: 206 - DNA, 21 bp, synthetic construct
  misc_feature 1..21: note = ma61134d04 Primer_Common
  source 1..21: mol_type = other DNA
gctcgctgctgcaccctcctt

(19) United States
(12) Patent Application Publication
(10) Pub. No.: US 2025/0179591 A1
(43) Pub. Date: Jun. 5, 2025

(54) PLANTS WITH IMPROVED DIGESTIBILITY AND MARKER HAPLOTYPES

(71) Applicant: KWS SAAT SE & Co. KGaA, Einbeck (DE)

(72) Inventors: Monika KLOIBER-MAITZ, Einbeck (DE); Nina MEYER, Einbeck (DE)

(73) Assignee: KWS SAAT SE & Co. KGaA, Einbeck (DE)

(21) Appl. No.: 18/292,125

(22) PCT Filed: Jul. 29, 2022
(86) PCT No.: PCT/EP2022/071303

§ 371 Date: Jan. 25, 2024

(30) Foreign Application Priority Data
Jul. 30, 2021 (EP) 21188948.0

(51) Int. Cl. C12Q 1/6895; C12N 15/82

(52) U.S. Cl. CPC C12Q 1/6895; C12N 15/8242; C12Q 2600/13; C12Q 2600/156

(57) ABSTRACT

The present invention relates to plants, in particular maize, having improved digestibility, in particular improved stover digestibility. The present invention relates to QTL alleles associated with improved digestibility and specific marker alleles associated with the QTL alleles. The present invention further relates to such plants, wherein the F35H gene is mutated or wherein F35H expression is reduced or absent. The invention also relates to methods for identifying plants having improved digestibility and methods for obtaining such plants.

### CROSS-REFERENCE TO RELATED APPLICATIONS

This application is a U.S. National Phase of International Patent Application No. PCT/EP2022/071303, filed on Jul. 29, 2022, which claims priority to European Application No. 21188948.0, filed Jul. 30, 2021. The entire contents of these applications are incorporated herein by reference in their entirety.

### SEQUENCE LISTING

The instant application contains a Sequence Listing which has been submitted electronically in XML file format and is hereby incorporated by reference in its entirety. Said XML copy, created on Jan. 3, 2025, is named 245761_000230_SL.xml and is 312,009 bytes in size.

### FIELD OF THE INVENTION

The invention relates to quantitative trait loci (QTL) and associated markers involved in and/or associated with improved digestibility of plants and plant parts, such as maize. The invention further relates to uses of such QTL or markers for identification and/or selection purposes, as well as transgenic or non-transgenic plants.

### BACKGROUND OF THE INVENTION

Maize (Zea mays L.) is the most important annual forage crop in the world. More than 3 million hectares of maize are ensiled each year, mainly in Northern Europe. Due to high energy content and feed conversion efficiency, the forage maize is an important food crop for dairy and beef cattle, and is affecting significantly the milk and meat production. There is a wide genetic variation in forage characteristics for both the entire maize plant and stover (Geiger et al. 1992; Barrière et al. 2003).

Therefore, improving digestibility is a major goal for forage maize breeding programs. The energy supplied by forage to a ruminant or herbivore animal diet is related to forage ingestibility and digestibility. The digestibility of any forage constituent (dry matter, organic matter, or cell wall) is measured as percentage of silage absorbed in the animal digestive tract (Barrière et al. 2003). The overall digestibility of forage maize is affected by the highly digestible grain and stover fraction. Stover composition and digestibility limits forage maize quality. Major stover fractions are hemicelluloses, cellulose, and lignins. Modern forage maize cultivars combine high dry matter yield with high stover digestibility.

It is too costly to perform digestibility measurements with animals, especially when conducting large scale evaluation of germplasm in plant breeding programs. Biological and chemical methods have been developed to assay the digestibility of maize and other forage crops (Van Soest et al. 1963). Neutral detergent fiber (NDF), the residual after removing cell soluble content, is an important plant cell wall and cellulose indicator. In vitro NDF digestibility (IVNDFD) of forages is an estimate of cell wall digestibility assuming that the non-NDF part of plant material was completely digestible (Méchin et al. 2000). Additionally, the use of NIRS has been reported to measure digestibility traits accurately in many forage crops including maize (Lübberstedt et al. 1997a, b; Zimmer et al. 1990).

Lübberstedt et al. (1997a, b) first published QTL related to forage maize agronomic and quality traits, and QTL for whole plant digestibility. Exploiting available genetic variation for stover digestibility by marker-assisted selection (MAS) seems to be a promising way to improve forage digestibility. Besides genetic variance, environmental variation might be the reason for those inconsistent traits. QTL analyses of forage traits in four different maize populations revealed only few QTL showing epistatic interactions or interactions with the environment (Lübberstedt et al. 1998). Seven QTL for DNDF were detected by using 242 RILs derived from the cross F838×F286 which were evaluated in per se value experiments in six environments, and found two major QTL (Barrière et al. 2010). Additional QTL analyses were conducted by using RIL progeny derived from a cross between an old dent and modern Iodent lines, and new QTL in bins 2.06 and 5.04 for ADL/NDF and DNDF were first reported (Barrière et al. 2012).

WO 2019/206927 describes a QTL for maize plant digestibility as well as the identification of a F35H gene linked to and responsible for the QTL for plant digestibility and description of a unique marker haplotype for improved digestibility. An insertion of about 187 nucleotides inside the F35H was found to be responsible for reduced activity of the enzyme which resulted in improved digestibility. Surprisingly, the inventors of present application found however, that the insertion behaves like a transposon. Transposons are known to change their location within the genome, the stability of this insertion is thus limited. Thereby the reliable use of the trait is not fully ensured.

It is therefore an objective of the present invention to address one or more of the shortcomings of the prior art. There is a persistent need for improving digestibility of fodder crops, as well as the identification of plants, including particular plant parts or derivatives having increased digestibility. In particular, it is an aim of the present invention to provide new and stable major QTL allele for digestibility and the provision of markers which allow the economical use of this QTL in maize development and breeding.

### SUMMARY OF THE INVENTION

The present invention is based on the identification of a major QTL for plant digestibility as well as the identification of new F35H alleles linked to and responsible for the QTL for plant digestibility and description of a unique marker haplotype for improved digestibility.

Molecular markers have been identified which are associated with plant digestibility, and marker alleles associated with improved digestibility are described.

The present inventors have found that the insertion in the allele previously described in WO 2019/206927 can behave like a transposon and can get lost, and excision might result in the loss of the knockout mutation causative for the improved digestibility trait. Indeed, according to studies by the present inventors based on 1720 doubled haploid (DH) lines of 42 populations analyzed for the presence of SILO-09-02, the digestibility improving QTL allele described in WO 2019/206927, the excision occurs in approximately 2% of all lines carrying the QTL allele. This shows that there is an unacceptable high risk to loss the trait.

The present inventors developed another QTL with a more advantageous type of insertion (7-8 base pairs) which is much more stable than the first one investigated in WO 2019/206927, and advantageously shows in parallel no yield penalty. The new insertions inside the F35H gene have now been described and markers for detection have been developed. Further, for the use of said new QTL in dent and flint pools for corn breeding, the present invention provides comprehensive set of claims allowing the conversion of any maize breeding pool with the newly developed QTL. The markers of the present invention are therefor uniquely suitable for detecting high digestibility phenotypes in many different maize lines/pools/haplotypes.

The invention in particular relates to methods for detecting the identified QTL allele associated with improved digestibility, as well as detection of any of the described marker alleles. The invention further relates to the described marker alleles and polynucleic acids useful for detection of the marker alleles, such as primers and probes, and kits comprising such. The invention further relates to methods for improving plant digestibility, in particular by naturally or artificially introducing in plants and/or selecting plants comprising the marker alleles described herein, such as in particular inducing F35H mutations, preferably mutations altering F35H expression or F35H enzymatic activity, e.g. reducing or eliminating F35H expression or F35H activity or otherwise reducing F35H expression or F35H activity, or increasing F35H activity. The invention further relates to plants having improved digestibility, as well as plant parts, in particular stover, having improved digestibility, such as seed deposited under NCIMB Deposit number NCIMB 43997.

The present invention is in particular captured by any one or any combination of one or more of the below numbered statements [1] to [91], with any other statement and/or embodiments.
[1] A method for identifying a maize plant or plant part, comprising screening for the presence of a polynucleotide comprising (molecular) marker (allele) ma61134d15 and/or ma61134d16 in (the genome of) a maize plant or plant part; wherein ma61134d15 is an insertion of 8 nucleotides between position 134254381 and 134254382 of chromosome 9 referenced to line PH207 or at position 76.19 cM on chromosome 9 referenced to line PH207, and/or detectable by molecular marker of SEQ ID NO: 125; preferably an insertion as set forth in SEQ ID NO: 1, and ma61134d16 is an insertion of 7 nucleotides between position 134254381 and 134254382 of chromosome 9 referenced to line PH207 or at position 76.19 cM on chromosome 9 referenced to line PH207, and/or detectable by molecular marker of SEQ ID NO: 124; preferably an insertion as set forth in SEQ ID NO: 4.
[2] The method according to statement [1], wherein said polynucleotide comprises or is comprised in a QTL (allele), in particular associated with improved digestibility, on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01; wherein wherein SYN38529 is a single nucleotide polymorphism (SNP) at position 56.38 cM on chromosome 9 referenced to line PH207, and/or detectable by molecular marker of SEQ ID NO: 13; PZE-109103504 is a single nucleotide polymorphism (SNP) at position 96.06 cM on chromosome 9 referenced to line PH207, and/or detectable by molecular marker of SEQ ID NO: 195; PZE-109076467 is a single nucleotide polymorphism (SNP) at position 75.85 cM on chromosome 9 referenced to line PH207, wherein said nucleotide is A or C, and/or detectable by molecular marker of SEQ ID NO: 109; and ma61161s01 is a single nucleotide polymorphism (SNP) at position 77.04 cM on chromosome 9 referenced to line PH207, wherein said nucleotide is A or G, and/or detectable by molecular marker of SEQ ID NO: 154.
[3] A method for identifying a maize plant or plant part, comprising screening for the presence of a QTL (allele), in particular associated with improved digestibility on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01; wherein wherein SYN38529 is a single nucleotide polymorphism (SNP) at position 56.38 cM on chromosome 9 referenced to line PH207, and/or detectable by molecular marker of SEQ ID NO: 13; PZE-109103504 is a single nucleotide polymorphism (SNP) at position 96.06 cM on chromosome 9 referenced to line PH207, and/or detectable by molecular marker of SEQ ID NO: 195; PZE-109076467 is a single nucleotide polymorphism (SNP) at position 75.85 cM on chromosome 9 referenced to line PH207, wherein said nucleotide is A or C, and/or detectable by molecular marker of SEQ ID NO: 109; and ma61161s01 is a single nucleotide polymorphism (SNP) at position 77.04 cM on chromosome 9 referenced to line PH207, wherein said nucleotide is A or G, and/or detectable by molecular marker of SEQ ID NO: 154.
[4] The method according to statement [3], wherein said QTL (allele) comprises (molecular) marker (allele) ma61134d15 and/or ma61134d16; wherein ma61134d15 is an insertion of 8 nucleotides between position 134254381 and 134254382 of chromosome 9 referenced to line PH207 or at position 76.19 cM on chromosome 9 referenced to line PH207, and/or detectable by molecular marker of SEQ ID NO: 125; preferably an insertion as set forth in SEQ ID NO: 1, and ma61134d16 is an insertion of 7 nucleotides between position 134254381 and 134254382 of 10 chromosome 9 referenced to line PH207 or at position 76.19 cM on chromosome 9 referenced to line PH207, and/or detectable by molecular marker of SEQ ID NO: 124; preferably an insertion as set forth in SEQ ID NO: 4.
[5] The method according to any of statements [1] to [4], comprising screening for the presence of one or more (molecular) marker (allele) selected from Table A.
[6] The method according to any of statements [1] to [4], comprising screening for the presence of one or more (molecular) marker (allele) selected from Table B.
[7] The method according to any of statements [1] to [4], comprising screening for the presence of one or more (molecular) marker (allele) selected from Table C.

20 [8] The method according to statement [1] or [2], wherein said polynucleotide comprises one or more (molecular) marker (allele) as defined in any of statements [5] to [7].
[9] The method according to statement [3] or [4], wherein said QTL (allele) comprises one or more (molecular) marker (allele) as defined in any of statements [5] to [7].
[10] A method for identifying a maize plant or plant part, comprising screening for or detecting the presence of one or more (molecular) marker (allele) as defined in statement [5] in (the genome of) a maize plant or plant part.
[11] The method according to statement [10], wherein said marker is comprised in a QTL, in particular associated with improved digestibility, on chromosome 9 flanked by and/or comprising (molecular) marker (allele) SYN38529 and PZE-109103504; wherein SYN38529 is a single nucleotide polymorphism (SNP) at position 56.38 cM on chromosome 9 referenced to line PH207, and/or detectable by molecular marker of SEQ ID NO: 13; and PZE-109103504 is a single nucleotide polymorphism (SNP) at position 96.06 cM on chromosome 9 referenced to line PH207, and/or detectable by molecular marker of SEQ ID NO: 195.
[12] A method for identifying a maize plant or plant part, comprising screening in (the genome of) a maize plant or plant part for the presence of a polynucleotide comprising a sequence selected from the group consisting of
   a) a nucleotide sequence of SEQ ID NO: 7;
   b) a nucleotide sequence having a coding sequence of SEQ ID NO: 8;
   c) a nucleotide sequence which is at least 90% identical to SEQ ID NO: 7 or 8;
   d) a nucleotide sequence encoding an amino acid sequence of SEQ ID NO: 9 or an amino acid sequence which is at least 90% identical to the sequence of SEQ ID NO: 9;
      wherein the nucleotide sequence has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8.
[13] The method according to statement [12], wherein the insertion of 8 nucleotides has a sequence of gcggttct, or wherein the insertion of 7 nucleotides has a sequence of gcggtct.
[14] The method according to statement [12] or [13], wherein the nucleotide sequence is selected from the group consisting of
   a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.
[15] A method for identifying a maize plant or plant part, comprising screening in (the genome of) a maize plant or plant part for the presence of a polynucleotide comprising a sequence selected from the group consisting of
   a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.
[16] The method according to any of statements [1] to [15], which is a method for identifying a plant or plant part having improved digestibility.
[17] The method according to any of statements [1] to [16], which is a method for identifying a plant or plant part having improved stover digestibility.
[18] The method according to statement [16] or [17], wherein said digestibility is improved compared to digestibility of maize line PH207 or to a (near) isogenic line which does not comprise the polynucleotide, the QTL (allele), or the one or more (molecular) marker (allele) of any of the preceding statements.
[19] The method according to statement [13], wherein said digestibility is improved compared to digestibility of maize line PH207 or to a (near) isogenic line which does not comprise the polynucleotide, the QTL (allele), or the one or more (molecular) marker (allele) of any of the preceding statements, by at least 5%, preferably at least 10%.
[20] The method according to any of statements [1] to [19], comprising isolating genomic DNA from the plant or plant part.
[21] The method according to any of statements [1] to [20], comprising selecting a plant of plant part comprising the polynucleotide, one or more of the (molecular) marker (allele), or the QTL (allele).
[22] The method according to any of statements [1] to [21], wherein said plant part is not propagation material.
[23] The method according to any of statements [1] to [22], wherein said plant part is stover.
[24] The method according to any of statements [1] to [23], wherein said plant or plant part comprises a polynucleotide having a sequence selected from
   a) a nucleotide sequence of SEQ ID NO: 7;
   b) a nucleotide sequence having a coding sequence of SEQ ID NO: 8;
   c) a nucleotide sequence which is at least 90% identical to SEQ ID NO: 7 or 8;
   d) a nucleotide sequence encoding an amino acid sequence of SEQ ID NO: 9 or an amino acid sequence which is at least 90% identical to the sequence of SEQ ID NO: 9;
   wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
   e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.
[25] The method according to any of statements [1] to [24], wherein said plant or plant part is identified as having improved digestibility if the polynucleotide, one or more of the (molecular) marker (allele), or QTL (allele) is present in the genome of said plant or plant part.
[26] The method according to any of statements [1] to [25], wherein said plant or plant part is identified as having improved stover digestibility if the polynucleotide, one or more of the (molecular) marker allele, or the QTL (allele) is present in the genome of said plant or plant part.
[27] A maize plant or plant part comprising one or more (molecular) marker (allele) as defined in Table A.
[28] A maize plant or plant part comprising one or more (molecular) marker (allele) as defined in Table B.
[29] A maize plant or plant part comprising one or more (molecular) marker (allele) as defined in Table C.
[30] A maize plant of plant part comprising the polynucleotide or the QTL (allele) as defined in any of statements [1] to [4], [8], [9] or to [15].
[31] The plant or plant part according to any of statements [27] to [30], wherein said plant or plant part is derived from a plant comprising said polynucleotide, said (molecular) marker (allele) or said QTL (allele) obtained by introduction or introgression.
[32] The plant or plant part according to any of statements [27] to [30], wherein said plant or plant part is obtained through mutagenesis mediated by transposon or transposable element.
[33] The plant or plant part according to any of statements [27] to [30], wherein said plant or plant part is transgenic or gene-edited.
[34] A method for generating or producing a maize plant or plant part and/or for improving (stover) digestibility, comprising introducing into the genome of a maize plant or plant part the polynucleotide, the one or more (molecular) marker (allele), or the QTL (allele) as defined in any of statements [1] to [7].
[35] A method for generating or producing a maize plant or plant part and/or for improving (stover) digestibility, comprising introducing into the genome of a maize plant or plant part a polynucleotide having a sequence selected from
   a) a nucleotide sequence of SEQ ID NO: 7;
   b) a nucleotide sequence having a coding sequence of SEQ ID NO: 8;
   c) a nucleotide sequence which is at least 90% identical to SEQ ID NO: 7 or 8;
   d) a nucleotide sequence encoding an amino acid sequence of SEQ ID NO: 9 or an amino acid sequence which is at least 90% identical to the sequence of SEQ ID NO: 9;
   wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
   e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.
[36] The method according to statement [34] or [35], wherein introducing into the genome comprises introgression.
[37] The method according to any of statements [34] to [36], comprising (a) providing a first maize plant having the polynucleotide, the one or more (molecular) marker (allele), or the QTL (allele) as defined in any of statements 1 to 9, or obtained from a Zea mays seed as deposited under NCIMB Deposit number NCIMB 43997 (or offspring thereof), (b) crossing said first maize plant with a second maize plant, and (c) selecting progeny plants having the polynucleotide, the one or more (molecular) marker (allele), or the QTL (allele) as defined in any of statements [1] to [9].
[38] The method according to statement [37], further comprising (d) harvesting a plant part from said progeny.
[39] The method according to statement or [35], wherein introducing into the genome comprises mutagenesis mediated by transposon or transposable element.
[40] The method according to statement or [35], wherein introducing into the genome comprises transgenesis or gene-editing.
[41] The method according to any of statements [34] to [40], wherein said plant part is a plant cell, tissue, organ, or seed.
[42] The method according to any of statements [34] to [41], wherein said plant part is a an immature or mature embryo, an inflorescence, a protoplast, a leaf, stover material, root material, a cobe, a seed, a kernel or a callus.
[43] The method according to any of statements [34], [35], or [39] to [42], comprising transforming a plant or plant part, preferably a plant cell, more preferably an immature or mature embryo, an inflorescence, a protoplast or callus, with said polynucleotide, said one or more (molecular) marker (allele), or said QTL (allele), and optionally regenerating a plant from said plant cell, preferably immature or mature embryo, inflorescence, protoplast or callus.
[44] The method according to any of statements [34] to [43], which is a method for improving digestibility of a plant or plant part.
[45] The method according to any of statements [34] to [44], which is a method for improving stover digestibility.
[46] The method according to any of statements [34] to [45], wherein said plant part is stover.
[47] A maize plant or plant part obtainable by the method according to any of statements [34] to [46].
[48] The method, plant or plant part according to any of statements [1] to [47], wherein said polynucleotide, said one or more (molecular) marker (allele), or said QTL (allele) is homozygous.
[49] The method, plant or plant part according to any of statements [1] to [47], wherein said polynucleotide, said one or more (molecular) marker (allele), or said QTL (allele) is heterozygous.
[50] Use of one or more molecular marker as defined in statement [5] for identifying or selecting a maize plant or plant part.
[51] Use of one or more molecular marker as defined in statement [6] for identifying or selecting a maize plant or plant part.
[52] Use of one or more molecular marker as defined in statement [7] for identifying or selecting a maize plant or plant part.
[53] Use according to any of statements [50] to [52], for identifying or selecting a maize plant or plant part having improved digestibility.
[54] Use according to any of statements [50] to [53] for identifying or selecting a maize plant or plant part having improved stover digestibility.
[55] Use according to any of statements [50] to [54], wherein said digestibility is improved compared to digestibility of maize line PH207 or to a (near) isogenic line which does not comprise the polynucleotide, the QTL (allele), the one or more (molecular) marker (allele) of any of the preceding statements.
[56] Use according to any of statements [50] to [55], wherein said digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.
[57] Use according to any of statements [50] to [56], wherein said plant part is stover.
[58] Use of the polynucleotide or the QTL (allele) as defined in any of statements 1 to 9 for generating or producing a maize plant or plant part.
[59] Use according to statement [58], for generating or producing a maize plant or plant part having improved digestibility.
[60] Use according to statement [58] or [59] for generating or producing a maize plant or plant part having improved stover digestibility.
[61] Use according to statement [59] or [60], wherein said digestibility is improved compared to digestibility of maize line PH207 or to a (near) isogenic line which does not comprise the polynucleotide, the QTL (allele), the one or more (molecular) marker (allele) of any of the preceding statements.
[62] Use according to any of statements [59] to [61], wherein said digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.
[63] Use according to any of statements [58] to [62], wherein said plant part is seed or stover.
[64] A (isolated) polynucleotide comprising a polynucleic acid having a sequence selected from
   a) a nucleotide sequence of SEQ ID NO: 7;
   b) a nucleotide sequence having a coding sequence of SEQ ID NO: 8;
   c) a nucleotide sequence which is at least 90% identical to SEQ ID NO: 7 or 8;
   d) a nucleotide sequence encoding an amino acid sequence of SEQ ID NO: 9 or an amino acid sequence which is at least 90% identical to the sequence of SEQ ID NO: 9;
   wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
   e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
   g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.
[65] Use of the (isolated) polynucleotide according to statement [64] for generating or producing a maize plant or plant part.
[66] Use according to statement [65], for generating or producing a maize plant or plant part having improved digestibility.
[67] Use according to statement [65] or [66] for generating or producing a maize plant or plant part having improved stover digestibility.
[68] Use according to statement [66] or [67], wherein said digestibility is improved compared to digestibility of maize line PH207 or to a (near) isogenic line which does not comprise the polynucleotide, the QTL (allele), the one or more (molecular) marker (allele) of any of the preceding statements.
[69] Use according to any of statements [66] to [68], wherein said digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.
[70] Use according to any of statements [65] to [69], wherein said plant part is stover.
[71] A (isolated) polynucleotide comprising a (molecular) marker (allele) as defined in statement [5], the complement or the reverse complement thereof, or a fragment thereof.
[72] A (isolated) polynucleotide comprising a (molecular) marker (allele) as defined in statement [6], the complement or the reverse complement thereof, or a fragment thereof.
[73] A (isolated) polynucleotide comprising a (molecular) marker (allele) as defined in statement [7], the complement or the reverse complement thereof, or a fragment thereof.
[74] The (isolated) polynucleotide according to any of statements [71] to [73], which comprises 10 to 500 nucleotides, preferably 15 to 250 nucleotides, more preferably 18 to 250 nucleotides, most preferably 20 to 250 nucleotides.
[75] The (isolated) polynucleic acid according to any of statements [71] to [74], in particular suitable as molecular marker, comprising at least 15, preferably at least 18, more preferably at least 20, contiguous nucleotides of any of SEQ ID NO: 13 to 195, or complementary to contiguous nucleotides of any of SEQ ID NO: 13 to 195, or reverse complementary to contiguous nucleotides of any of SEQ ID NO: 13 to 195, and preferably comprising at least one nucleotide of the respective polymorphism (donor allele) as provided in Table A.
[76] The (isolated) polynucleic acid according to any of statements [71] to [74], in particular suitable as molecular marker, comprising at least 15, preferably at least 18, more preferably at least 20, contiguous nucleotides of any of SEQ ID NO: 109 to 154, or complementary to contiguous nucleotides of any of SEQ ID NO: 109 to 154, or reverse complementary to contiguous nucleotides of any of SEQ ID NO: 109 to 154, and preferably comprising at least one nucleotide of the respective polymorphism (donor allele) as provided in Table A.
[77] The (isolated) polynucleic acid according to any of statements [71] to [74], in particular suitable as molecular marker, comprising at least 15, preferably at least 18, more preferably at least 20, contiguous nucleotides of any of SEQ ID NO: 124 to 125, or complementary to contiguous nucleotides of any of SEQ ID NO: 124 to 125, or reverse complementary to contiguous nucleotides of any of SEQ ID NO: 124 to 125, and preferably comprising at least one nucleotide of the respective polymorphism (donor allele) as provided in Table A.
[78] The (isolated) polynucleotide according to any of statements [71] to [78], which is specific to a plant or plant part having improved stover digestibility.
[79] A (isolated) polynucleic acid specifically hybridizing with a molecular marker as defined in statement [5], the complement or the reverse complement thereof.
[80] A (isolated) polynucleic acid specifically hybridizing with a molecular marker as defined in statement [6], the complement or the reverse complement thereof.
[81] A (isolated) polynucleic acid specifically hybridizing with a molecular marker as defined in statement [7], the complement or the reverse complement thereof.
[82] The (isolated) polynucleic acid according to any of statements [71] to [81], which is a primer or a probe.
[83] The (isolated) polynucleic acid according to any of statements [71] to [82], which is an allele-specific primer.
[84] The (isolated) polynucleic acid according to any of statements [71] to [83], which is a KASP primer.
[85] A primer or probe capable of specifically detecting the polynucleotide, the one or more (molecular) marker (allele), or the QTL (allele) as defined in any of statements [1] to [9].
[86] A primer set capable of specifically detecting the polynucleotide, the one or more (molecular) marker (allele), or the QTL (allele) as defined in any of statements [1] to [9].
[87] A Zea mays seed as deposited under NCIMB Deposit number NCIMB 43997.
[88] A (K0001) Zea mays seed, a representative sample of which has been deposited under NCIMB Deposit No. NCIMB 43997.
[89] A Zea mays plant grown or obtained from the seed according to statement 87 or 88, or offspring thereof.
[90] A Zea mays plant part grown or obtained from the seed according to statement [87] or [88] or obtained from the plant (or offspring thereof) according to statement [89].
[91] The Zea mays plant part according to statement [90], wherein said plant part is stover.

### BRIEF DESCRIPTION OF THE FIGURES

FIG. 1: DNDF (Digestible Neutral Detergent Fiber) on inbred line level of maize (Zea mays). Analysis has been performed using a NIRS calibration as described further above. Left column: wildtype without 7 bp insertion; right column inbred line with 7 bp insertion according to an embodiment of the invention.

FIG. 2: Total Dry Matter Yield (TDY) in dt/ha of four maize hybrids No-Ins1, No-Ins2, Ins1 and Ins2. No-Ins1 and No-Ins2 do not have an insertion in the F35H gene. Ins1 and Ins2 are heterozygous for the 7 base pair insertion in the F35H gene according to an embodiment of the invention.

FIG. 3: Total Dry Matter Yield (TDY) relative to control mean (rcm) in percent of four maize hybrids No-Ins1, No-Ins2, Ins1 and Ins2. No-Ins1 and No-Ins2 do not have an insertion in the F35H gene. Ins1 and Ins2 are heterozygous for the 7 base pair insertion in the F35H gene according to an embodiment of the invention.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

As used herein, “maize” refers to a plant of the species Zea mays, preferably Zea mays ssp mays.

The term “plant” includes whole plants, including descendants or progeny thereof. The term “plant part” includes any part or derivative of the plant, including particular plant tissues or structures, plant cells, an immature or mature plant embryo, an plant inflorescence, a plant callus, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. In certain embodiments, the plant part or derivative as referred to herein is stover. In certain embodiments, the plant part or derivative as referred to herein is a seed or a part of a seed.

As used herein, the term plant population may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

Stover as used herein has its ordinary meaning known in the art. By means of further guidance, and without limitation, stover may comprise, consist of, or consist essentially of the leaves and stalks of field crops, such as maize that are commonly left in a field after harvesting the grain. Stover may also include cobs (e.g. the central core of an ear of maize, without the kernels). Stover may also exclude cobs. Stover may also include husks or hulls (e.g. the leafy outer covering of an ear of maize). Stover may also exclude husks or hulls. Stover is similar to straw, the residue left after any cereal grain or grass has been harvested at maturity for its seed. It can be directly grazed by cattle or dried for use as fodder. (Maize) stover can be used as feed, whether grazed as forage, chopped as silage to be used later for fodder, or collected for direct (non-ensilaged) fodder use. Maize forage is usually ensiled in cooler regions, but it can be harvested year-round in the tropics and fed as green forage to the animals. In the silage use case, it is usual for the entire plant (grain and stover together) to be chopped into pieces which are then crushed between rollers while harvesting. In addition to the stalks, leaves, husks, and cobs remaining in the field, kernels of grain may also be left over from harvest. These left over kernels, along with the corn stover, serve as an additional feed source for grazing cattle.

In certain embodiments, the plant part or derivative comprises, consists of, or consists essentially of one or more, preferably all of stalks, leaves, and cobs. In certain embodiments, the plant part or derivative is leaves. In certain embodiments, the plant part or derivative is stalks. In certain embodiments, the plant part or derivative is cobs. In certain embodiments, the plant part or derivative comprises, consists of, or consists essentially of one or more, preferably all of stalks and leaves. In certain embodiments, the plant part or derivative comprises, consists of, or consists essentially of one or more, preferably all of stalks, and cobs. In certain embodiments, the plant part or derivative comprises, consists of, or consists essentially of one or more, preferably all of leaves and cobs. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, ensilaging, etc.

As used herein, “digestibility” refers to and is measured as percentage of product (such as a maize plant or plant part or derivative, including for instance dry matter, organic matter, or cell wall of the product) absorbed in the animal digestive tract (Barrière et al. 2003). Biological and chemical methods have been developed to assay the digestibility of maize and other forage crops (Van Soest et al. 1963). Neutral detergent fiber (NDF), the residual after removing cell soluble content, is an important plant cell wall and cellulose indicator. In vitro NDF digestibility (IVNDFD) of forages is an estimate of cell wall digestibility assuming that the non-NDF part of plant material was completely digestible (Méchin et al. 2000). Additionally, the use of NIRS has been reported to measure digestibility traits accurately in many forage crops including maize (Lübberstedt et al. 1997a, b; Zimmer et al. 1990). In certain embodiments, the animal is a mammal. In certain embodiments, the animal is a ruminant. In certain embodiments, the animal is a herbivore. In certain embodiments, the animal is a herbivorous mammal.

“Improved digestibility” as referred to herein, relates to increased digestibility of a plant or plant part, such as stover, or derivative having a characteristic according to the invention, such as a polynucleotide, mutation, marker, SNP, or QTL as described herein elsewhere, compared to a plant or plant part or derivative not having such characteristic, such as a reference plant (or plant part). In certain embodiments, an improved or increased (stover) digestibility refers to an increase in mean DNDF by at least 1%, such as at least 2%, at least 3%, at least 4%, at least 5%, at least 6%, at least 7%, at least 8%, at least 9%, or at least 10%, preferably at least 2%, more preferably at least 3%, such as at least 4%, most preferably at least 5%, such as at least 10%. In certain embodiments, the reference plant (or plant part) is the maize inbred line PH207, as described in “Draft Assembly of Elite Inbred Line PH207 Provides Insights into Genomic and Transcriptome Diversity in Maize”, Hirsch et al., Plant Cell. 2016 November; 28 (11): 2700-2714, published online 2016 Nov. 1. doi: 10.1105/tpc.16.00353, or a or to a (near) isogenic line which does not comprise the polynucleotide, mutation, marker, SNP, or QTL of the invention as described herein elsewhere. The skilled person will understand that in the context of a plant part other than stover, such as for instance a seed, when reference is made to improved stover digestibility it is to be understood that such refers to stover of the plant from which such plant part is derived. For instance, reference to a seed having “improved stover digestibility” relates to stover from the plant from which the seed is derived and/or stover from the plant grown from the seed, preferably at least stover from the plant grown from the seed.

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more monomorphic and/or polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL, polynucleotide, marker, etc. of the invention as described herein may be homozygous. In certain embodiments, the QTL, polynucleotide, marker, etc. of the invention as described herein may be heterozygous.

As used herein, the term “allele” or “alleles” refers to one or more alternative forms, i.e. different nucleotide sequences, of a locus.

As used herein, the term “mutant alleles” or “mutation” of alleles include alleles having one or more mutations, such as insertions, deletions, stop codons, base changes (e.g., transitions or transversions), or alterations in splice junctions/splicing signal sites, which may or may not give rise to altered gene products. Modifications in alleles may arise in coding or non-coding regions (e.g. promoter regions, exons, introns or splice junctions).

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 50 Mb or less, such as about 30 Mb or less, about 20 Mb or less, about 25 Mb or less, about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, a locus, an introgression fragment, a QTL, or a gene or allele conferring a trait (such as improved digestibility) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, QTL, gene or allele. The terms are used interchangeably and the genetic element, locus, introgression fragment, QTL, gene or allele can thus be transferred into any other genetic background lacking the trait. Not only pants comprising the genetic element, locus, introgression fragment, QTL, gene or allele can be used, but also progeny/descendants from such plants which have been selected to retain the genetic element, locus, introgression fragment, QTL, gene or allele, can be used and are encompassed herein. Whether a plant (or genomic DNA, cell or tissue of a plant) comprises the same genetic element, locus, introgression fragment, QTL, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic or gene-edited plants may also be encompassed.

As used herein the terms “genetic engineering”, “transformation” and “genetic or transgenic modification” are all used herein as synonyms for the transfer of isolated and cloned genes into the DNA, usually the chromosomal DNA or genome, of another organism.

“Introducing” in the meaning of the present invention includes stable or transient integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cas12), CRISPR/CasX, or CRISPR/CasY), TALENs, zinc finger nucleases or meganucleases, homologous recombination optionally by means of one of the below mentioned gene editing technology including preferably a repair template, modification of endogenous gene using random or targeted mutagenesis like TILLING or above mentioned gene editing technology, etc.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “exogene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”. “Endogene” refers to a nucleic acid molecule or a genetic locus that naturally occurs in the genome of a plant.

“Gene editing” or “genome editing” refers to genetic engineering in which in which DNA or RNA is inserted, deleted, modified or replaced in the genome of an organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas) system. These nucleases create site-specific double-strand breaks (DSBs) at desired locations in the genome. The induced double-strand breaks are repaired through nonhomologous end-joining (NHEJ) or homologous recombination (HR) or homology directed repair (HDR), resulting in targeted mutations or nucleic acid modifications. The use of designer nucleases is particularly suitable for generating gene knockouts or knockdowns. In certain embodiments, designer nucleases are developed which specifically induce a mutation in the F35H gene, as described herein elsewhere, such as to generate a mutated F35H or a knockout of the F35H gene. In certain embodiments, designer nucleases, in particular RNA-specific CRISPR/Cas systems are developed which specifically target the F35H mRNA, such as to cleave the F35H mRNA and generate a knockdown of the F35H gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence. Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and/or downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and downstream of the corresponding region in the genome of a plant not comprising the QTL allele, this region is excised and can be replaced with the template comprising the QTL allele of the invention. In this way, introduction of the QTL allele of the invention in a plant need not involve multiple backcrossing, in particular in a plant of specific genetic background. Similarly, the mutated F35H of the invention may be provided on/as a template. More advantageously however, the mutated F35H of the invention may be generated without the use of a recombination template, but solely through the endonuclease action leading to a double strand DNA break which is repaired by NHEJ, resulting in the generation of indels.

In certain embodiments, the nucleic acid modification or mutation is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39: e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35) z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WC 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; US application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339 (6121): 819-23 (2013); RNA-guided editing of bacterial genomes using CRISPR-Cas systems. Jiang W., Bikard D., Cox D., Zhang F, Marraffini L A. Nat Biotechnol March; 31 (3): 233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153 (4): 910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500 (7463): 472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674 (13) 01015-5. (2013); DNA targeting specificity of RNA-guided Cas9 nucleases. Hsu, P., Scott, D., Weinstein, J., Ran, FA., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013); Genome engineering using the CRISPR-Cas9 system. Ran, FA., Hsu, P D., Wright, J., Agarwala, V., Scott, DA., Zhang, F. Nature Protocols November; 8 (11): 2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156 (5): 935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889; CRISPR-Cas9 Knockin Mice for Genome Editing and Cancer Modeling, Platt et al., Cell 159 (2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343 (6166): 80-84. doi: 10.1126/science. 1246981; Rational design of highly active sgRNAs for CRISPR-Cas9-mediated gene inactivation, Doench et al., Nature Biotechnology 32 (12): 1262-7 (2014) published online 3 Sep. 2014; doi: 10.1038/nbt.3026, and In vivo interrogation of gene function in the mammalian brain using CRISPR-Cas9, Swiech et al, Nature Biotechnology 33, 102-106 (2015) published online 19 Oct. 2014; doi: 10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60 (3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In a particular embodiment, a mutated nucleic acid-targeting effector protein based on CRISPR system as described above which lacks the ability to cleave one or both DNA or RNA strands of a target polynucleotide containing a target sequence can be fused to other tools like other nucleases, nickases, recombinases, transposases, base editors or molecular complexes including these tools. A “base editor” as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest. Preferably, the at least one base editor in the context of the present invention is temporarily or permanently linked to at least one site-specific effector, or optionally to a component of at least one site-specific effector complex (e.g., DNA recognition domain of CRISPR system, zinc finger or TAL effectors). The linkage can be covalent and/or non-covalent.

Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA′R-loop′ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al ((2017). Programmable base editing of A·T to G·C in genomic DNA without DNA cleavage. Nature, 557 (7681), 464.) described adenine base editors (ABEs) that mediate the conversion of A·T to G*C in genomic DNA.

Any base editing complex according to the present invention can thus comprise at least one cytidine deaminase, or a catalytically active fragment thereof. The at least one base editing complex can comprise the cytidine deaminase, or a domain thereof in the form of a catalytically active fragment, as base editor.

In another embodiment, the at least one first targeted base modification is a conversion of any nucleotide C, A, T, or G, to any other nucleotide. Any one of a C, A, T or G nucleotide can be exchanged in a site-directed way as mediated by a base editor, or a catalytically active fragment thereof, to another nucleotide. The at least one base editing complex can thus comprise any base editor, or a base editor domain or catalytically active fragment thereof, which can convert a nucleotide of interest into any other nucleotide of interest in a targeted way. In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523 (7561): 481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18 (4): 455-7 and McCallum et al. “Targeting induced local lesions IN genomes (TILLING) for plant functional genomics”; Plant Physiol. 2000 June; 123 (2): 439-42.

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC). Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase III enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. As used herein, an RNAi molecule may be an siRNA, shRNA, or a miRNA. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the QTL and/or one or more marker(s) as described herein is/are homozygous. In certain embodiments, the QTL and/or one or more marker(s) as described herein are heterozygous. In certain embodiments, the QTL allele, polynucleotide, and/or one or more marker(s) allele(s) as described herein is/are homozygous. In certain embodiments, the QTL allele, polynucleotide, and/or one or more marker(s) allele(s) as described herein are heterozygous.

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “(molecular) marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant. As used herein, the term (molecular) marker allele may be used interchangeably with “donor allele” or “allele donor”, and is meant to be the (molecular) marker allele associated with improved digestibility according to the invention, unless explicitly indicated otherwise.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a digestibility QTL as defined herein, can be used to detect and/or select plants comprising the QTL on chromosome 9. The closer the genetic linkage of the molecular marker to the locus (e.g. about 7 cM, 6 CM, 5 CM, 4 cM, 3 CM, 2 cM, 1 cM, 0.5 CM or less), the less likely it is that the marker is dissociated from the locus through meiotic recombination. Likewise, the closer two markers are linked to each other (e.g. within 7 cM or 5 CM, 4 CM, 3 CM, 2 cM, 1 cM or less) the less likely it is that the two markers will be separated from one another (and the more likely they will co-segregate as a unit). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the B12seq program can be found in the readme file accompanying BLASTZ. BI2seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq 1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\B12seq -i c:\seq1 .txt -j c:\seq2.txt -p blastn -o c:\output.txt -q -1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the B12seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly<->Ala, Val< >Ile<->Leu, Asp<->Glu, Lys<->Arg, Asn<->Gln or Phe<->Trp<->Tyr. Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

As used herein, the term “endogenous” refers to a gene or allele which is present in its natural genomic location. The term “endogenous” can be used interchangeably with “native” or “wild-type”. This does not however exclude the presence of one or more nucleic acid differences with the wild-type allele. In particular embodiments, the difference with a wild-type allele can be limited to less than 9 preferably less than 6, more particularly less than 3 nucleotide differences, such as 0 nucleotides difference. More particularly, the difference with the wildtype sequence can be in only one nucleotide. Preferably, the endogenous allele encodes a modified protein having less than 9, preferably less than 6, more particularly less than 3 and even more preferably only one or no amino acid difference with the wild-type protein.

A used herein, the term “exogenous polynucleotide” refers to a polynucleotide, such as a gene (or cDNA) or allele which is or has been recombinantly introduced in a cell (or plant). The exogenous polynucleotide may be episomal or genomically integrated. Integration may be random or site-directed. Integration may include replacement of a corresponding endogenous polynucleotide. It will be understood that an exogenous polynucleotide is not naturally present in the cell or plant.

When reference is made to a nucleic acid sequence (e.g. DNA or genomic DNA) having “substantial sequence identity to” a reference sequence or having a sequence identity of at least 60%>, e.g. at least 70%, 75%, 80%, 85%, 90%, 95%, 98% or 99% nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001). Examples of high stringent hybridization conditions are conditions under which primarily only those nucleic acid molecules that have at least 90% or at least 95% sequence identity undergo hybridization. Such high stringent hybridization conditions are, for example: 4×SSC at 65° C. and subsequent multiple washes in 0.1×SSC at 65° C. for approximately 1 hour. The term “high stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions. Less stringent hybridization conditions are, for example: hybridizing in 4×SSC at 37° C. and subsequent multiple washing in 1×SSC at room temperature.

As used herein, degenerate nucleotides are referred to according to IUPAC nucleotide code standards.

As used herein, F35H (ExPASy enzyme entry EC 1.14.13.88) refers to the flavonoid 3′,5′-hydroxylase gene or protein. F35H is also known as F3′5′H, F3′,5′H, cytochrome P450 flavonoid 3′,5′-hydroxylase, or flavanone, NADPH: oxygen oxidoreductase. F35H catalyzes the following reaction: flavanone+2 NADPH+2 O(2)<=>3′,5′-dihydroxyflavanone+2 NADP (+)+2 H(2)O.

The present invention relates to maize plants or plant parts, such as stover, characterized by having specific (molecular) marker (alleles), in particular associated with improved (stover) digestibility. The invention further relates to the use of such markers for generating as well as identifying or selecting maize plants or plant parts. The invention also relates to (isolated) polynucleic acids comprising such markers or suitable for identifying or detecting such markers.

Suitable markers for use according to certain embodiments of the present invention are provided in Table A below.

TABLE A

| | | donor | | |
|---|---|---|---|---|
| | | (improved digestibility) | | |
| | | allele | | |
| SEQ | | Polymorphism | Poly- | |
| ID | | position in | morphism | |
| NO: | Marker | SEQ ID NO | identity | Comment |
| 13 | SYN38529 | 61 | | polymorphic |
| 14 | SYN5866 | 61 | | polymorphic |
| 15 | SYN11764 | 61 | | polymorphic |
| 16 | PZE-109015136 | 51 | | polymorphic |
| 17 | PZE-109015397 | 51 | | polymorphic |
| 18 | PZE-109016177 | 51 | | polymorphic |
| 19 | PZE-109017276 | 51 | | polymorphic |
| 20 | PZE-109017407 | 51 | | polymorphic |
| 21 | PZE-109018614 | 51 | | polymorphic |
| 22 | SYN32168 | 61 | | polymorphic |
| 23 | PZE-109021152 | 51 | | polymorphic |
| 24 | PZE-109021389 | 51 | | polymorphic |
| 25 | zm06133s02 | 101 | | polymorphic |
| 26 | PZE-109022641 | 51 | | polymorphic |
| 27 | Affx-90814890 | 31 | | polymorphic |
| 28 | SYN34181 | 61 | | polymorphic |
| 29 | SYN29297 | 61 | | polymorphic |
| 30 | PZE-109026962 | 51 | | polymorphic |
| 31 | SYN13048 | 61 | | polymorphic |
| 32 | PZE-109063861 | 51 | | polymorphic |
| 33 | PZE-109029427 | 51 | | polymorphic |
| 34 | zm08444s04 | 101 | | polymorphic |
| 35 | zm08444s03 | 101 | | polymorphic |
| 36 | PZE-109031125 | 51 | | polymorphic |
| 37 | PZE-109033123 | 51 | | polymorphic |
| 38 | SYN18776 | 61 | | polymorphic |
| 39 | PZE-109035519 | 51 | | polymorphic |
| 40 | PZE-109035852 | 51 | | polymorphic |
| 41 | PZE-109036977 | 51 | | polymorphic |
| 42 | zm10851s01 | 101 | | polymorphic |
| 43 | PZE-109041138 | 51 | | polymorphic |
| 44 | PZE-109040213 | 51 | | polymorphic |
| 45 | PZE-109043990 | 51 | | polymorphic |
| 46 | PZE-109045856 | 51 | | polymorphic |
| 47 | PZE-109048885 | 51 | | polymorphic |
| 48 | SYN32492 | 101 | | polymorphic |
| 49 | SYN32488 | 61 | | polymorphic |
| 50 | zm11758s02 | 101 | | polymorphic |
| 51 | PZE-109051621 | 51 | | polymorphic |
| 52 | PZE-109053851 | 51 | | polymorphic |
| 53 | PZE-109053885 | 51 | | polymorphic |
| 54 | PZE-109053887 | 51 | | polymorphic |
| 55 | PZE-109053906 | 51 | | polymorphic |
| 56 | PZE-109054050 | 51 | | polymorphic |
| 57 | PZE-109054055 | 51 | | polymorphic |
| 58 | PZE-109054205 | 51 | | polymorphic |
| 59 | PZE-109054458 | 51 | | polymorphic |
| 60 | PZE-109054723 | 51 | | polymorphic |
| 61 | PZE-109054729 | 51 | | polymorphic |
| 62 | PZE-109054805 | 51 | | polymorphic |
| 63 | PZE-109054812 | 51 | | polymorphic |
| 64 | PZE-109054943 | 51 | | polymorphic |
| 65 | SYN32299 | 61 | | polymorphic |
| 66 | PZE-109058460 | 51 | | polymorphic |
| 67 | Affx-91355778 | 36 | | polymorphic |
| 68 | PZE-109061769 | 51 | | polymorphic |
| 69 | SYN36472 | 61 | | polymorphic |
| 70 | PUT-163a- | 56 | | polymorphic |
| | 4646495- | | | |
| | 2099 | | | |
| 71 | PZE-109065712 | 51 | | polymorphic |
| 72 | Affx-91059234 | 36 | | polymorphic |
| 73 | PZE-109066188 | 51 | | polymorphic |
| 74 | PZE-109067522 | 51 | | polymorphic |
| 75 | PZE-109067146 | 51 | | polymorphic |
| 76 | PZE-109066949 | 51 | | polymorphic |
| 77 | PZE-109066773 | 51 | | polymorphic |
| 78 | PZE-100001123 | 51 | | polymorphic |
| 79 | PZE-100000965 | 51 | | polymorphic |
| 80 | ZM011231-0277 | 61 | | polymorphic |
| 81 | PZE-109067561 | 51 | | polymorphic |
| 82 | PZE-109067890 | 51 | | polymorphic |
| 83 | PZE-109068071 | 51 | | polymorphic |
| 84 | SYN21877 | 61 | | polymorphic |
| 85 | PZE-109071755 | 51 | | polymorphic |
| 86 | PZE0008250961 | 51 | | polymorphic |
| 87 | SYN29013 | 61 | | polymorphic |
| 88 | PZE-109072798 | 51 | | polymorphic |
| 89 | SYN23066 | 101 | | polymorphic |
| 90 | PZE-109073024 | 51 | | polymorphic |
| 91 | PZE-109073894 | 51 | | polymorphic |
| 92 | SYN30845 | 61 | | polymorphic |
| 93 | ma60054s01 | 101 | | polymorphic |
| 94 | PZE-109074314 | 51 | | polymorphic |
| 95 | SYN22542 | 61 | | polymorphic |
| 96 | PZE-109074671 | 51 | | polymorphic |
| 97 | PZE-109074676 | 51 | | polymorphic |
| 98 | PZE0004090171 | 51 | | polymorphic |
| 99 | PZE-100001246 | 51 | | polymorphic |
| 100 | PZE-100001337 | 51 | | polymorphic |
| 101 | PZE-100001338 | 51 | | polymorphic |
| 102 | SYN4635 | 61 | | polymorphic |
| 103 | PZE-109075140 | 51 | | polymorphic |
| 104 | PZE-109075353 | 51 | | polymorphic |
| 105 | PZE-109075487 | 51 | | polymorphic |
| 106 | PZE-109075564 | 51 | | polymorphic |
| 107 | PZE-109075835 | 51 | | polymorphic |
| 108 | PZE-109075980 | 51 | | polymorphic |
| 109 | PZE-109076467 | 51 | ade | |
| 110 | PZE-109076558 | 51 | gua | |
| 111 | PZE-109076739 | 51 | ade | |
| 112 | PZE-109076761 | 51 | ade | |
| 113 | PZE-109077374 | 51 | ade | |
| 114 | ma61126d01 | 100-101 | | deletion of |
| | | | | SEQ ID |
| | | | | NO: 115 |
| | | | | between |
| | | | | position |
| | | | | 100-101 of |
| | | | | SEQ ID |
| | | | | NO: 114 |
| 116 | PZE-109077509 | 51 | ade | |
| 117 | PZE-109077598 | 51 | gua | |
| 118 | PZE-109077976 | 51 | ade | |
| 119 | PZE-109078320 | 51 | ade | |
| 120 | ma60405s01 | 101 | gua | |
| 121 | PZE-109078814 | 51 | gua | |
| 122 | PZE-109078789 | 51 | cyt | |
| 123 | PZE-109079170 | 51 | ade | |
| 124 | ma61134d16 | 101-107 | gcggtct | 7 bp insertion |
| 125 | ma61134d15 | 101-108 | gcggttct | 8 bp insertion |
| 126 | Affx-91110408 | 101 | ade | |
| 127 | PUT-163a- | 56 | cyt | |
| | 16926216- | | | |
| | 1143 | | | |
| 128 | Affx-91121762 | 36 | ade | |
| 129 | PZE-109080508 | 51 | ade | |
| 130 | PZE-109080569 | 51 | gua | |
| 131 | ma60182s01 | 101 | ade | |
| 132 | PZE-109080822 | 51 | ade | |
| 133 | ma61125s01 | 101 | ade | |
| 134 | SYN29273 | 61 | ade | |
| 135 | PZE-109081405 | 51 | gua | |
| 136 | PZE-109081479 | 51 | ade | |
| 137 | Affx-90937015 | 36 | thy | |
| 138 | PZE-109081680 | 51 | gua | |
| 139 | SYN29834 | 61 | gua | |
| 140 | PZE-109082046 | 51 | gua | |
| 141 | PZE-109082004 | 51 | gua | |
| 142 | Affx-90383113 | 36 | cyt | |
| 143 | SYN34099 | 61 | gua | |
| 144 | Affx-91186901 | 10 | gua | |
| 145 | SYN33659 | 61 | ade | |
| 146 | PZE-109082918 | 51 | ade | |
| 147 | PZE-109083369 | 51 | ade | |
| 148 | PZE-109083328 | 51 | gua | |
| 149 | SYN29817 | 61 | gua | |
| 150 | ma60360s01 | 101 | ade | |
| 151 | ma61158s01 | 101 | thy | |
| 152 | ma61036s01 | 101 | thy | |
| 153 | PZE-109086494 | 51 | cyt | |
| 154 | ma61161s01 | 101 | ade | |
| 155 | ma60806s03 | 101 | | polymorphic |
| 156 | ma61071s01 | 101 | | polymorphic |
| 157 | SYN38267 | 61 | | polymorphic |
| 158 | PZE-109088808 | 51 | | polymorphic |
| 159 | PUT-163a- | 55 | | polymorphic |
| | 60348412- | | | |
| | 2592 | | | |
| 160 | PHM1599.84 | 61 | | polymorphic |
| 161 | PZE-109090188 | 51 | | polymorphic |
| 162 | ma60984s01 | 101 | | polymorphic |
| 163 | PZE-109091377 | 51 | | polymorphic |
| 164 | PZE-109092033 | 51 | | polymorphic |
| 165 | PZE-109092182 | 51 | | polymorphic |
| 166 | SYN27192 | 61 | | polymorphic |
| 167 | PZE-109093491 | 51 | | polymorphic |
| 168 | PZE-109093846 | 51 | | polymorphic |
| 169 | PZE-109094238 | 51 | | polymorphic |
| 170 | PZE-109094399 | 51 | | polymorphic |
| 171 | ZM013688-0470 | 20 | | polymorphic |
| 172 | PZE-109096103 | 51 | | polymorphic |
| 173 | SYN22829 | 61 | | polymorphic |
| 174 | SYN22625 | 61 | | polymorphic |
| 175 | PZE-109097654 | 51 | | polymorphic |
| 176 | PZE-109097752 | 51 | | polymorphic |
| 177 | PZE-109098108 | 51 | | polymorphic |
| 178 | zm00728s04 | 101 | | polymorphic |
| 179 | PZE-109098623 | 51 | | polymorphic |
| 180 | SYN27711 | 61 | | polymorphic |
| 181 | SYN27709 | 61 | | polymorphic |
| 182 | PZE-109099670 | 51 | | polymorphic |
| 183 | Affx-91229917 | 36 | | polymorphic |
| 184 | PZE-109100755 | 51 | | polymorphic |
| 185 | SYN25124 | 61 | | polymorphic |
| 186 | SYN25123 | 61 | | polymorphic |
| 187 | PZE-109101695 | 51 | | polymorphic |
| 188 | PZE-109101698 | 51 | | polymorphic |
| 189 | PZE-109102054 | 51 | | polymorphic |
| 190 | PZE-109102157 | 51 | | polymorphic |
| 191 | zm02908s02 | 101 | | polymorphic |
| 192 | Affx-90454668 | 36 | | polymorphic |
| 193 | PUT-163a- | 56 | | polymorphic |
| | 60357079- | | | |
| | 2822 | | | |
| 194 | Affx-90988936 | 36 | | polymorphic |
| 195 | PZE-109103504 | 51 | | polymorphic |

Preferred suitable markers for use according to certain embodiments of the present invention are provided in Table B below.

TABLE B

| | | donor | | |
|---|---|---|---|---|
| | | (improved digestibility) | | |
| | | allele | | |
| SEQ | | Polymorphism | Poly- | |
| ID | | position in | morphism | |
| NO: | Marker | SEQ ID NO | identity | Comment |
| 109 | PZE-109076467 | 51 | ade | |
| 110 | PZE-109076558 | 51 | gua | |
| 111 | PZE-109076739 | 51 | ade | |
| 112 | PZE-109076761 | 51 | ade | |
| 113 | PZE-109077374 | 51 | ade | |
| 114 | ma61126d01 | 100-101 | | deletion of |
| | | | | SEQ ID |
| | | | | NO: 115 |
| | | | | between |
| | | | | position |
| | | | | 100-101 of |
| | | | | SEQ ID |
| | | | | NO: 114 |
| 116 | PZE-109077509 | 51 | ade | |
| 117 | PZE-109077598 | 51 | gua | |
| 118 | PZE-109077976 | 51 | ade | |
| 119 | PZE-109078320 | 51 | ade | |
| 120 | ma60405s01 | 101 | gua | |
| 121 | PZE-109078814 | 51 | gua | |
| 122 | PZE-109078789 | 51 | cyt | |
| 123 | PZE-109079170 | 51 | ade | |
| 124 | ma61134d16 | 101-107 | gcggtct | 7 bp insertion |
| 125 | ma61134d15 | 101-108 | gcggttct | 8 bp insertion |
| 126 | Affx-91110408 | 101 | ade | |
| 127 | PUT-163a- | 56 | cyt | |
| | 16926216-1143 | | | |
| 128 | Affx-91121762 | 36 | ade | |
| 129 | PZE-109080508 | 51 | ade | |
| 130 | PZE-109080569 | 51 | gua | |
| 131 | ma60182s01 | 101 | ade | |
| 132 | PZE-109080822 | 51 | ade | |
| 133 | ma61125s01 | 101 | ade | |
| 134 | SYN29273 | 61 | ade | |
| 135 | PZE-109081405 | 51 | gua | |
| 136 | PZE-109081479 | 51 | ade | |
| 137 | Affx-90937015 | 36 | thy | |
| 138 | PZE-109081680 | 51 | gua | |
| 139 | SYN29834 | 61 | gua | |
| 140 | PZE-109082046 | 51 | gua | |
| 141 | PZE-109082004 | 51 | gua | |
| 142 | Affx-90383113 | 36 | cyt | |
| 143 | SYN34099 | 61 | gua | |
| 144 | Affx-91186901 | 10 | gua | |
| 145 | SYN33659 | 61 | ade | |
| 146 | PZE-109082918 | 51 | ade | |
| 147 | PZE-109083369 | 51 | ade | |
| 148 | PZE-109083328 | 51 | gua | |
| 149 | SYN29817 | 61 | gua | |
| 150 | ma60360s01 | 101 | ade | |
| 151 | ma61158s01 | 101 | thy | |
| 152 | ma61036s01 | 101 | thy | |
| 153 | PZE-109086494 | 51 | cyt | |
| 154 | ma61161s01 | 101 | ade | |

Most preferred suitable markers for use according to certain embodiments of the present invention are provided in Table C below.

TABLE C

| | | donor (improved digestibility) | | |
|---|---|---|---|---|
| | | allele | | |
| | | Polymorphism | | |
| SEQ ID | | position in | Polymorphism | |
| NO: | Marker | SEQ ID NO | identity | Comment |
| 124 | ma61134d16 | 101-107 | gcsgtct | 7 bp insertion |
| 125 | ma61134d15 | 101-108 | gcsgttct | 8 bp insertion |

In an aspect, the invention relates to a method for identifying or selecting a maize plant or plant part, in particular a maize plant or plant part having improved digestibility, preferably improved stover digestibility, comprising screening for the presence of or identifying a QTL on chromosome 9 in (the genome, such as in isolated genetic material from the plant or plant part, of) a maize plant or plant part, or fragment thereof, in particular associated with improved (stover) digestibility, flanked by SYN38529 and PZE-109103504, preferably PZE-109076467 and ma61161s01 or comprised in a polynucleotide or QTL (allele) comprising and/or flanked by (molecular) marker (alleles) SYN38529 and PZE-109103504, preferably PZE-109076467 and ma61161s01.

As referred to herein, a polynucleic acid, such as for instance a QTL (allele) as described herein, is said to be flanked by certain molecular markers or molecular marker alleles if the polynucleic acid is comprised within a polynucleic acid wherein respectively a first marker (allele) is located upstream (i.e. 5′) of said polynucleic acid and a second marker (allele) is located downstream (i.e. 3′) of said polynucleic acid. Such first and second marker (allele) may border the polynucleic acid. The nucleic acid may equally comprise such first and second marker (allele), such as respectively at or near the 5′ and 3′ end, for instance respectively within 50 kb of the 5′ and 3′ end, preferably within 10 kb of the 5′ and 3′ end, such as within 5 kb of the 5′ and 3′ end, within 1 kb of the 5′ and 3′ end, or less.

The skilled person will understand that detecting any of the (molecular) marker (allele) of Tables A, B, or C may equivalently detect any of the other (molecular) marker (allele), given their close linkage. For example, screening for a polynucleotide comprising ma61134d16 or ma61134d15 may be done by screening for these marker(s) (allele(s)) themselves, or alternatively by screening for any of the other marker (allele) in Table A or B, preferable Table B.

In certain embodiments, ma61134d16 or ma61134d15 are not both present in the polynucleotide or QTL (allele) of the invention as described herein elsewhere.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a QTL on chromosome 9 in (the genome, such as in isolated genetic material from the plant or plant part, of) a maize plant or plant part, or fragment thereof, in particular associated with improved (stover) digestibility, flanked by SYN38529 and PZE-109103504, preferably PZE-109076467 and ma61161s01 or comprised in a polynucleotide or QTL (allele) comprising and/or flanked by (molecular) marker (alleles) SYN38529 and PZE-109103504, preferably PZE-109076467 and ma61161s01.

In an embodiment, the invention relates to a method for identifying or selecting a maize plant or plant part, in particular a maize plant or plant part having improved digestibility, preferably improved stover digestibility, comprising screening for the presence of or identifying a QTL on chromosome 9 in (the genome, such as in isolated genetic material from the plant or plant part, of) a maize plant or plant part, or fragment thereof, in particular associated with improved (stover) digestibility, flanked by SYN38529 and PZE-109103504, preferably PZE-109076467 and ma61161s01 or comprised in a polynucleotide or QTL (allele) comprising and/or flanked by (molecular) marker (alleles) SYN38529 and PZE-109103504, preferably PZE-109076467 and ma61161s01, and selecting a plant or plant part in which the QTL is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a QTL on chromosome 9 in (the genome, such as in isolated genetic material from the plant or plant part, of) a maize plant or plant part, or fragment thereof, in particular associated with improved (stover) digestibility, flanked by SYN38529 and PZE-109103504, preferably PZE-109076467 and ma61161s01 or comprised in a polynucleotide or QTL (allele) comprising and/or flanked by (molecular) marker (alleles) SYN38529 and PZE-109103504, preferably PZE-109076467 and ma61161s01, and selecting a plant or plant part in which the QTL is present.

Preferably, the QTL comprises one or more (molecular) marker (alleles) of Table A. More preferably, the QTL comprises one or more (molecular) marker (alleles) of Table B. Most preferably, the QTL comprises one or more (molecular) marker (alleles) of Table C.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table A in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table A in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table A in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table A in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table B in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table B in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table B in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table B in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table C in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table C in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more (molecular) marker (allele) of Table C in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more (molecular) marker (allele) of Table C in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more of SEQ ID NOs: 13-195 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 13-195 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or of SEQ ID NOs: 13-195 (having the marker (donor) allele at the indicated position in Table A) in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 13-195 (having the marker (donor) allele at the indicated position in Table A) in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more of SEQ ID NOs: 109-154 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 109-154 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more of SEQ ID NOs: 109-154 (having the marker (donor) allele at the indicated position in Table A) in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 109-154 (having the marker (donor) allele at the indicated position in Table A) in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more of SEQ ID NOs: 124-125 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 124-125 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising one or more of SEQ ID NOs: 124-125 (having the marker (donor) allele at the indicated position in Table A) in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising one or more of SEQ ID NOs: 124-125 (having the marker (donor) allele at the indicated position in Table A) in (the genome, such as in isolated genetic material from the plant or plant part, of) a maize plant or plant part, and selecting a plant or plant part in which the polynucleotide is present.

In an aspect, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising (molecular) marker (allele) ma61134d15 and/or ma61134d16 in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising (molecular) marker (allele) ma61134d15 and/or ma61134d16 in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying a polynucleotide, such as a QTL or fragment thereof, in particular associated with improved (stover) digestibility, comprising (molecular) marker (allele) ma61134d15 and/or ma61134d16 in (the genome, such as in isolated genetic material from the plant or plant part, of) a maize plant or plant part, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying a polynucleotide, such as a QTL or fragment thereof, in particular associated with improved (stover) digestibility, comprising (molecular) marker (allele) ma61134d15 and/or ma61134d16 in (the genome, such as in isolated genetic material from the plant or plant part, of) a maize plant or plant part, and selecting a plant or plant part in which the polynucleotide is present.

In certain of the above embodiments, screening for the presence of or identifying/detecting the polynucleotide comprises screening for the presence of or identifying/detecting any of the indicated (molecular) marker (alleles) or SEQ ID NOs.

In certain embodiments, the polynucleotide or QTL (allele) is located on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01.

In an aspect, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table A in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table A in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table A in (the genome, such as in isolated genetic material from the plant or plant part, of) a maize plant or plant part, and selecting a plant or plant part in which the (molecular) marker (allele) is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table A in (the genome, such as in isolated genetic material from the plant or plant part, of) a maize plant or plant part, and selecting a plant or plant part in which the (molecular) marker (allele) is present.

In an aspect, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table B in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table B in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table B in (the genome, such as in isolated genetic material from the plant or plant part, of) a maize plant or plant part, and selecting a plant or plant part in which the (molecular) marker (allele) is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table B in (the genome, such as in isolated genetic material from the plant or plant part, of) a maize plant or plant part, and selecting a plant or plant part in which the (molecular) marker (allele) is present.

In an aspect, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table C in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table C in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or more (molecular) marker (allele) of Table C in (the genome, such as in isolated genetic material from the plant or plant part, of) a maize plant or plant part, and selecting a plant or plant part in which the (molecular) marker (allele) is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more (molecular) marker (allele) of Table C in (the genome, such as in isolated genetic material from the plant or plant part, of) a maize plant or plant part, and selecting a plant or plant part in which the (molecular) marker (allele) is present.

In an aspect, the invention relates to a method for identifying or selecting a maize plant or plant part, in particular a maize plant or plant part having improved digestibility, preferably improved stover digestibility, comprising screening for the presence of or identifying one or more of SEQ ID NOs: 13-195 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 13-195 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or of SEQ ID NOs: 13-195 (having the marker (donor) allele at the indicated position in Table A) in (the genome, such as in isolated genetic material from the plant or plant part, of) a maize plant or plant part, and selecting a plant or plant part in which the SEQ ID NO is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 13-195 (having the marker (donor) allele at the indicated position in Table A) in (the genome, such as in isolated genetic material from the plant or plant part, of) a maize plant or plant part, and selecting a plant or plant part in which the SEQ ID NO is present.

In an aspect, the invention relates to a method for identifying or selecting a maize plant or plant part, in particular a maize plant or plant part having improved digestibility, preferably improved stover digestibility, comprising screening for the presence of or identifying one or more of SEQ ID NOs: 109-154 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 109-154 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or more of SEQ ID NOs: 109-154 (having the marker (donor) allele at the indicated position in Table A) in (the genome, such as in isolated genetic material from the plant or plant part, of) a maize plant or plant part, and selecting a plant or plant part in which the SEQ ID NO is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 109-154 (having the marker (donor) allele at the indicated position in Table A) in (the genome, such as in isolated genetic material from the plant or plant part, of) a maize plant or plant part, and selecting a plant or plant part in which the SEQ ID NO is present.

In an aspect, the invention relates to a method for identifying or selecting a maize plant or plant part, in particular a maize plant or plant part having improved digestibility, preferably improved stover digestibility, comprising screening for the presence of or identifying one or more of SEQ ID NOs: 124-125 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 124-125 (having the marker (donor) allele at the indicated position in Table A) in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying one or more of SEQ ID NOs: 124-125 (having the marker (donor) allele at the indicated position in Table A) in (the genome, such as in isolated genetic material from the plant or plant part, of) a maize plant or plant part, and selecting a plant or plant part in which the SEQ ID NO is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying one or more of SEQ ID NOs: 124-125 (having the marker (donor) allele at the indicated position in Table A) in (the genome, such as in isolated genetic material from the plant or plant part, of) a maize plant or plant part, and selecting a plant or plant part in which the SEQ ID NO is present.

In an aspect, the invention relates to a method for identifying or selecting a maize plant or plant part, in particular a maize plant or plant part having improved digestibility, preferably improved stover digestibility, comprising screening for the presence of or identifying (molecular) marker (allele) ma61134d15 and/or ma61134d16 in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying (molecular) marker (allele) ma61134d15 and/or ma61134d16 in (the genome, such as in isolated genetic material from the plant or plant part, of) a maize plant or plant part.

In an embodiment, the invention relates to a method for identifying or selecting a maize plant or plant part, in particular a maize plant or plant part having improved digestibility, preferably improved stover digestibility, comprising screening for the presence of or identifying (molecular) marker (allele) ma61134d15 and/or ma61134d16 in (the genome, such as in isolated genetic material from the plant or plant part, of) a maize plant or plant part, and selecting a plant or plant part in which the (molecular) marker (allele) is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying (molecular) marker (allele) ma61134d15 and/or ma61134d16 in (the genome, such as in isolated genetic material from the plant or plant part, of) a maize plant or plant part, and selecting a plant or plant part in which the (molecular) marker (allele) is present.

In certain embodiments of the identification methods of the invention, the maize plant or plant part comprises a polynucleic acid comprising one or more (molecular marker (allele) of Table A. In certain embodiments of the identification methods of the invention, the maize plant or plant part comprises a polynucleic acid comprising one or more (molecular marker (allele) of Table B. In certain embodiments of the identification methods of the invention, the maize plant or plant part comprises a polynucleic acid comprising one or more (molecular marker (allele) of Table C.

In certain embodiments of the identification methods of the invention, the maize plant or plant part comprises a polynucleic acid comprising a polynucleic acid having a sequence as set forth in SEQ ID NO: 1 or 4. In certain embodiments of the identification methods of the invention, the maize plant or plant part comprises a polynucleic acid having a coding sequence or encoding a polynucleic acid having a sequence as set forth in SEQ ID NO: 2 or 5. In certain embodiments of the identification methods of the invention, the maize plant or plant part comprises a polynucleic acid comprising a polynucleic acid encoding a polypeptide having a sequence as set forth in SEQ ID NO: 3 or 6.

In an aspect, the invention relates to a method for identifying or selecting a maize plant or plant part, in particular a maize plant or plant part having improved digestibility, preferably improved stover digestibility, comprising screening for the presence of or identifying in (the genome, such as in isolated genetic material from the plant or plant part, of) a maize plant or plant part a polynucleotide comprising a sequence selected from
a) a nucleotide sequence of SEQ ID NO: 7;
b) a nucleotide sequence having a coding sequence of SEQ ID NO: 8;
c) a nucleotide sequence which is at least 90%, preferably at least 95%, identical to SEQ ID NO: 7 or 8;
d) a nucleotide sequence encoding an amino acid sequence of SEQ ID NO: 9 or an amino acid sequence which is at least 90%, preferably at least 95%, identical to the sequence of SEQ ID NO: 9;
wherein the nucleotide sequence has an insertion of one or more nucleotides, preferably not a multiple of three, more preferably resulting in a frame shift, most preferably of 8 or 7 nucleotides at a position corresponding to the position between thymine at position 102 and guanine at position 103 of SEQ ID NO: 7 or the corresponding position in SEQ ID NO: 8.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying in (the genome, such as in isolated genetic material from the plant or plant part, of) a maize plant or plant part a polynucleotide comprising a sequence selected from
a) a nucleotide sequence of SEQ ID NO: 7;
b) a nucleotide sequence having a coding sequence of SEQ ID NO: 8;
c) a nucleotide sequence which is at least 90%, preferably at least 95%, identical to SEQ ID NO: 7 or 8;
d) a nucleotide sequence encoding an amino acid sequence of SEQ ID NO: 9 or an amino acid sequence which is at least 90%, preferably at least 95%, identical to the sequence of SEQ ID NO: 9;
wherein the nucleotide sequence has an insertion of one or more nucleotides, preferably not a multiple of three, more preferably resulting in a frame shift, most preferably of 8 or 7 nucleotides at a position corresponding to the position between thymine at position 102 and guanine at position 103 of SEQ ID NO: 7 or the corresponding position in SEQ ID NO: 8.

In an embodiment, the invention relates to a method for identifying or selecting a maize plant or plant part, in particular a maize plant or plant part having improved digestibility, preferably improved stover digestibility, comprising screening for the presence of or identifying in (the genome, such as in isolated genetic material from the plant or plant part, of) a maize plant or plant part a polynucleotide comprising a sequence selected from
a) a nucleotide sequence of SEQ ID NO: 7;
b) a nucleotide sequence having a coding sequence of SEQ ID NO: 8;
c) a nucleotide sequence which is at least 90%, preferably at least 95%, identical to SEQ ID NO: 7 or 8;
d) a nucleotide sequence encoding an amino acid sequence of SEQ ID NO: 9 or an amino acid sequence which is at least 90%, preferably at least 95%, identical to the sequence of SEQ ID NO: 9;
wherein the nucleotide sequence has an insertion of one or more nucleotides, preferably not a multiple of three, more preferably resulting in a frame shift, most preferably of 8 or 7 nucleotides at a position corresponding to the position between thymine at position 102 and guanine at position 103 of SEQ ID NO: 7 or the corresponding position in SEQ ID NO: 8, and selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying in (the genome, such as in isolated genetic material from the plant or plant part, of) a maize plant or plant part a polynucleotide comprising a sequence selected from
a) a nucleotide sequence of SEQ ID NO: 7;
b) a nucleotide sequence having a coding sequence of SEQ ID NO: 8;
c) a nucleotide sequence which is at least 90%, preferably at least 95%, identical to SEQ ID NO: 7 or 8;
d) a nucleotide sequence encoding an amino acid sequence of SEQ ID NO: 9 or an amino acid sequence which is at least 90%, preferably at least 95%, identical to the sequence of SEQ ID NO: 9;
wherein the nucleotide sequence has an insertion of one or more nucleotides, preferably not a multiple of three, more preferably resulting in a frame shift, most preferably of 8 or 7 nucleotides at a position corresponding to the position between thymine at position 102 and guanine at position 103 of SEQ ID NO: 7 or the corresponding position in SEQ ID NO: 8, and selecting a plant or plant part in which the polynucleotide is present.

The skilled person will understand that corresponding nucleotide positions can be determined by suitable alignment, as is known in the art.

In certain embodiments, the insertion of 7 nucleotides has a sequence of gcggtct.

In certain embodiments, the insertion of 8 nucleotides has a sequence of gcggttct.

In certain embodiments, the polynucleotide has a sequence selected from
a) a nucleotide sequence of SEQ ID NO: 7;
b) a nucleotide sequence having a coding sequence of SEQ ID NO: 8;
c) a nucleotide sequence encoding an amino acid sequence of SEQ ID NO: 9;
wherein the nucleotide sequence has an insertion of 8 or 7 nucleotides at a position corresponding to the position between thymine at position 102 and guanine at position 103 of SEQ ID NO: 7.

In certain embodiments, the polynucleotide has a sequence selected from
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In certain embodiments, the polynucleotide has a sequence selected from:
a) a nucleotide sequence of SEQ ID NO: 1 or 4;
b) a nucleotide sequence having a coding sequence of SEQ ID NO: 2 or 5;
c) a nucleotide sequence encoding an amino acid sequence of SEQ ID NO: 3 or 6.

In an aspect, the invention relates to a method for identifying or selecting a maize plant or plant part, in particular a maize plant or plant part having improved digestibility, preferably improved stover digestibility, comprising screening for the presence of or identifying in (the genome, such as in isolated genetic material from the plant or plant part, of) a maize plant or plant part a polynucleotide comprising a sequence selected from
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying in (the genome, such as in isolated genetic material from the plant or plant part, of) a maize plant or plant part a polynucleotide comprising a sequence selected from
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In an embodiment, the invention relates to a method for identifying or selecting a maize plant or plant part, in particular a maize plant or plant part having improved digestibility, preferably improved stover digestibility, comprising screening for the presence of or identifying in (the genome, such as in isolated genetic material from the plant or plant part, of) a maize plant or plant part a polynucleotide comprising a sequence selected from
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1, and
selecting a plant or plant part in which the polynucleotide is present.

In an embodiment, the invention relates to a method for identifying or selecting a maize plant or plant part, in particular a maize plant or plant part having improved digestibility, preferably improved stover digestibility, comprising isolating genetic material from at least one cell of the plant or plant part, and screening for the presence of or identifying in (the genome, such as in isolated genetic material from the plant or plant part, of) a maize plant or plant part a polynucleotide comprising a sequence selected from
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcgtct, at a position corresponding respectively to position 103-109 of SEQ ID NO: 4, or gcsgttct, preferably gcggttct, at a position corresponding to position 103-110 of SEQ ID NO: 1, and
selecting a plant or plant part in which the polynucleotide is present.

In certain embodiments, screening for the presence or identifying comprises for the presence or identifying the 7 bp insert having a sequence of gcggtct (corresponding to position 103-109 of SEQ ID NO: 4).

In certain embodiments, screening for the presence or identifying comprises for the presence or identifying the 8 bp insert having a sequence of gcggttct (corresponding to position 103-110 of SEQ ID NO: 1).

SEQ ID NOs: 1 and 4 correspond to the genomic sequence of the mutated F35H gene according to certain embodiments of the invention, and respectively comprising an 8 bp insert or a 7 bp insert, as described herein elsewhere.

SEQ ID NOs: 2 and 5 correspond to the coding sequence of the mutated F35H gene according to certain embodiments of the invention, and respectively comprising an 8 bp insert or a 7 bp insert, as described herein elsewhere (i.e. at positions corresponding to the indicated genomic sequence).

SEQ ID NOs: 3 and 6 correspond to the polypeptide sequence encoded by the mutated F35H gene according to certain embodiments of the invention, and respectively comprising an 8 bp insert or a 7 bp insert, as described herein elsewhere.

It will be understood that in the methods as described above, when the, polynucleotide, QTL allele, (molecular) marker allele, or SEQ ID NO is present or identified/detected, then the plant or plant part is identified as having improved digestibility, in particular improved stover digestibility. Accordingly, in certain embodiments, the methods for identifying a plant or plant part as described above are methods for identifying a plant or plant part having improved digestibility, in particular improved stover digestibility. Alternatively, in the methods as described above, when the, polynucleotide, QTL allele, (molecular) marker allele, or SEQ ID NO is not present or identified/detected, then the plant or plant part is identified as not having improved digestibility, in particular not having improved stover digestibility.

Methods for screening for or detecting the presence of a polynucleic acid, QTL allele, (molecular) marker allele, or SEQ ID NO as described herein are known in the art. Without limitation, screening or detecting may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc. Preferably the one or more polynucleic acid, QTL allele, (molecular) marker allele, or SEQ ID NO is detectable by a polynucleic acid, such as an allele specific polynucleic acid (molecular marker), suitable for hybridization as forward primer or reverse primer to a locus in the chromosomal interval which co-segregates with the improved digestibility.

In an aspect, the invention relates to a maize plant or plant part identified or selected by the methods of the invention as described herein elsewhere.

In an aspect, the invention relates to a method, such as a method for generating or producing a maize plant or plant part, in particular a plant or plant part having improved digestibility, preferably improved stover digestibility, comprising introducing into the genome of a maize plant or plant part a polynucleotide, (molecular) marker (allele), QTL (allele) or mutated F35H gene of the invention as described herein elsewhere.

In an aspect, the invention relates to a method for generating or producing a maize plant or plant part, in particular a plant or plant part having improved digestibility, preferably improved stover digestibility, comprising introducing into the genome of a maize plant or plant part a polynucleotide, (molecular) marker (allele), QTL (allele) or mutated F35H gene of the invention as described herein elsewhere.

In an aspect, the invention relates to a method for improving digestibility of a maize plant or plant part, preferably stover digestibility, comprising introducing into the genome of a maize plant or plant part a polynucleotide, (molecular) marker (allele), QTL (allele) or mutated F35H gene of the invention as described herein elsewhere.

In an aspect, the invention relates to a method, such as a method for generating a plant or plant part and/or improving digestibility of a plant or plant part, preferably stover digestibility, comprising introducing into the genome of a plant a QTL (allele), in particular associated with improved (stover) digestibility and comprising a nucleotide sequence encoding a cytochrome P450 flavonoid 3′,5′-hydroxylase having a mutation.

In an aspect, the invention relates to a method, such as a method for generating a plant or plant part and/or for improving digestibility of a plant or plant part, preferably stover digestibility, comprising introducing into the genome of a plant a QTL (allele) comprising a nucleotide sequence encoding a cytochrome P450 flavonoid 3′,5′-hydroxylase having a mutation.

In certain embodiments, the QTL (allele) is located on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01.

In certain embodiments, the QTL (allele) is located on chromosome 9 and comprises one or more of (molecular) marker (alleles) selected from Table A.

In certain embodiments, the QTL (allele) is located on chromosome 9 and comprises one or more of (molecular) marker (alleles) selected from Table B.

In certain embodiments, the QTL (allele) is located on chromosome 9 and comprises one or more of (molecular) marker (alleles) selected from Table C.

In an aspect, the invention relates to a method, such as a method for generating a plant or plant part and/or improving digestibility of a plant or plant part, preferably stover digestibility, comprising introducing into the genome of a plant a a polynucleotide comprising (molecular) marker (allele) selected from Table A, preferably all (optionally except for one of ma61134d15 or ma61134d16).

In an aspect, the invention relates to a method, such as a method for generating a plant or plant part and/or improving digestibility of a plant or plant part, preferably stover digestibility, comprising introducing into the genome of a plant a a polynucleotide comprising (molecular) marker (allele) selected from Table B, preferably all (optionally except for one of ma61134d15 or ma61134d16).

In an aspect, the invention relates to a method, such as a method for generating a plant or plant part and/or improving digestibility of a plant or plant part, preferably stover digestibility, comprising introducing into the genome of a plant a a polynucleotide comprising (molecular) marker (allele) selected from Table C.

In an aspect, the invention relates to a method, such as a method for generating a plant or plant part and/or improving digestibility of a plant or plant part, preferably stover digestibility, comprising introducing into the genome of a plant a a polynucleotide comprising (molecular) marker (allele) ma61134d15 and/or ma61134d16 in (the genome of) a maize plant or plant part.

In an aspect, the invention relates to a method, such as a method for generating a plant or plant part and/or improving digestibility of a plant or plant part, preferably stover digestibility, comprising introducing into the genome of a plant a polynucleotide having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In certain embodiments, digestibility is improved compared to digestibility of maize line PH207 or to a (near) isogenic line which does not comprise the polynucleotide, the QTL (allele), or the one or more (molecular) marker (allele) of any of the preceding statements.

In certain embodiments, digestibility is improved compared to digestibility of maize line PH207 or to a (near) isogenic line which does not comprise the polynucleotide, the QTL (allele), or the one or more (molecular) marker (allele) of any of the preceding statements, by at least 5%, preferably at least 10%.

In certain embodiments, the polynucleotide or QTL (allele) comprises one or more of the marker alleles of the invention as described herein elsewhere.

In certain embodiments, the polynucleotide or QTL (allele) comprises a mutated F35H gene as described herein elsewhere.

In certain embodiments, the invention relates to a method for obtaining or generating a maize plant or plant part, comprising (a) providing a first plant having a polynucleotide, such as a QTL (allele), such as a QTL (allele) associated with improved (stover) digestibility as described herein elsewhere, optionally wherein said QTL (allele) is located on a chromosomal interval, preferably on chromosome 9, and comprising and/or flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01, or said first plant being obtained from a Zea mays seed as deposited under NCIMB Deposit number NCIMB 43997 (or offspring thereof), (b) crossing said first plant with a second plant, such as a second plant not having said polynucleotide, such as said QTL (allele), (c) selecting progeny plants having said polynucleotide, such as said QTL (allele), and optionally (d) harvesting said plant part from said progeny.

In certain embodiments, the invention relates to a method for obtaining or generating a maize plant or plant part, comprising (a) providing a first plant having a (molecular) marker allele, such as a (molecular) marker allele associated with improved (stover) digestibility selected from Table A; (b) crossing said first plant with a second plant, such as a second plant not having said (molecular) marker allele, (c) selecting progeny plants having said (molecular) marker allele, and optionally (d) harvesting said plant part from said progeny.

In certain embodiments, the invention relates to a method for obtaining or generating a maize plant or plant part, comprising (a) providing a first plant having a (molecular) marker allele, such as a (molecular) marker allele associated with improved (stover) digestibility selected from Table B; (b) crossing said first plant with a second plant, such as a second plant not having said (molecular) marker allele, (c) selecting progeny plants having said (molecular) marker allele, and optionally (d) harvesting said plant part from said progeny.

In certain embodiments, the invention relates to a method for obtaining or generating a maize plant or plant part, comprising (a) providing a first plant having a (molecular) marker allele, such as a (molecular) marker allele associated with improved (stover) digestibility selected from Table C; (b) crossing said first plant with a second plant, such as a second plant not having said (molecular) marker allele, (c) selecting progeny plants having said (molecular) marker allele, and optionally (d) harvesting said plant part from said progeny.

In certain embodiments, the polynucleotide, QTL (allele), (molecular) marker (allele), and/or F35H mutation in the first plant is present in a homozygous state. In certain embodiments the polynucleotide, QTL (allele), (molecular) marker (allele), and/or F35H mutation in the first plant is present in a heterozygous state. In certain embodiments the polynucleotide, QTL (allele), (molecular) marker (allele), and/or F35H mutation in the second plant is present in a heterozygous state. In certain embodiments the polynucleotide, QTL (allele), (molecular) marker (allele), and/or F35H mutation in the second plant is not present.

In certain embodiments, the progeny is selected in which the polynucleotide, QTL (allele), (molecular) marker (allele), and/or mutated F35H of the invention is present in a homozygous state.

In certain embodiments, the progeny is selected in which the polynucleotide, QTL (allele), (molecular) marker (allele), and/or mutated F35H of the invention is present in a heterozygous state.

In certain embodiments, the invention relates to a method for obtaining or generating a maize plant or plant part, comprising transforming a maize plant or plant part, preferably a plant cell, more preferably an immature or mature embryo, an inflorescence, a protoplast or callus, with said polynucleotide, said one or more (molecular) marker (allele), or said QTL (allele), and optionally regenerating a plant from said plant cell, preferably immature or mature embryo, inflorescence, protoplast or callus.

As described herein elsewhere, in certain embodiments the maize plant or plant part does not comprise endogenously the recited polynucleic acids, (molecular) marker (allele), QTL (allele) and/or mutated F35H.

In certain embodiments, the methods for obtaining plants or plant parts as described herein according to the invention, such as the methods for obtaining plants or plant parts having improved digestibility, involve or comprise transgenesis and/or gene editing and/or base editing, such as including CRISPR/Cas, TALEN, ZFN, meganucleases; (induced) mutagenesis, which may or may not be random mutagenesis, such as TILLING. In certain embodiments, the methods for obtaining plants or plant parts as described herein according to the invention, such as the methods for obtaining plants or plant parts having improved digestibility, involve or comprise RNAi applications, which may or may not be, comprise, or involve transgenic applications. By means of example, non-transgenic applications may for instance involve applying RNAi components such as double stranded siRNAs to plants or plant surfaces, such as for instance as a spray. Stable integration into the plant genome is not required.

In certain embodiments, the methods for obtaining plants or plant parts as described herein according to the invention, such as the methods for obtaining plants or plant parts having improved digestibility, do not involve or comprise transgenesis, gene/base editing, and/or mutagenesis.

In certain embodiments, the methods for obtaining plants or plant parts as described herein according to the invention, such as the methods for obtaining plants or plant parts having improved digestibility, involve, comprise or consist of breeding and selection.

In certain embodiments, the methods for obtaining plants or plant parts as described herein according to the invention, such as the methods for obtaining plants or plant parts having improved digestibility, do not involve, comprise or consist of breeding and selection.

Knockdown or knockout of F35H may be effected by any of the mutagenesis methods described herein.

The skilled person will understand that the introduction or mutation as described herein of two or more sequences, genes, markers, or alleles can be simultaneous or sequential, and that the introduction or mutation as described herein of two or more sequences, genes, markers, or alleles can be both recombinantly (e.g. through transformation) or by breeding techniques (e.g. introgression), or that introduction of one of the two or more sequences, genes, markers, or alleles can be recombinantly and another by breeding techniques (e.g. introgression).

In an aspect, the invention relates to a maize plant or plant part obtained or obtainable by the methods of the invention as described herein, such as the methods for generating, producing, or obtaining plants or plant parts, in particular plants or plant parts having improved (stover) digestibility. The invention also relates to the progeny of such plants.

In an aspect, the invention relates to a plant or plant part comprising a polynucleotide, such as a QTL (allele), in particular associated with improved (stover) digestibility, said polynucleotide, such as QTL (allele) comprising a nucleotide sequence of a gene encoding a cytochrome P450 flavonoid 3′,5′-hydroxylase (F35H) having a mutation.

In certain embodiments, the QTL (allele) is located on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01.

In certain embodiments, the QTL (allele) is located on chromosome 9 and comprises one or more of (molecular) marker (alleles) of Table A8, preferably all (optionally except for one of ma61134d15ma61134d15 or ma61134d16).

In certain embodiments, the QTL (allele) is located on chromosome 9 and comprises one or more of (molecular) marker (alleles) of Table B, preferably all (optionally except for one of ma61134d15ma61134d15 or ma61134d16).

In certain embodiments, the QTL (allele) is located on chromosome 9 and comprises one or more of (molecular) marker (alleles) of Table C.

In an aspect, the invention relates to a plant or plant part comprising a nucleotide sequence of a gene encoding a cytochrome P450 flavonoid 3′,5′-hydroxylase (F35H) having a mutation.

In an aspect, the invention relates to a plant or plant part comprising one or more (molecular) marker (allele) selected from Table A, preferably all (optionally except for one of ma61134d15 or ma61134d16).

In an aspect, the invention relates to a plant or plant part comprising one or more (molecular) marker (allele) selected from Table B, preferably all (optionally except for one of ma61134d15 or ma61134d16).

In an aspect, the invention relates to a plant or plant part comprising one or more (molecular) marker (allele) selected from Table C, preferably all.

In an aspect, the invention relates to a plant or plant part comprising
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcgttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

The skilled person will understand that the recited polynucleic acids may be operatively linked to one or more regulatory sequences, in particular regulatory sequences effecting transcriptions of said polynucleotide, in particular the coding region of said polynucleotide. Such regulatory sequences may include suitable (plant) promoters, transcription initiation sites, transcription termination sites, etc., as is known in the art.

In certain embodiments, the plant is not a plant variety.

In certain embodiments, the wild type or unmutated F35H gene comprises
(i) a nucleotide sequence comprising the sequence of SEQ ID NO: 7;
(ii) a nucleotide sequence having the cDNA or coding sequence of SEQ ID NO: 8;
(iii) a nucleotide sequence encoding for a polypeptide having the amino acid sequence of SEQ ID NO: 9;
(iv) a nucleotide sequence having at least 60% identity to the sequence of SEQ ID NO: 7, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;
(v) a nucleotide sequence encoding for a polypeptide having at least 60% identity to the sequence of SEQ ID NO: 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the polypeptide encoded by the nucleotide sequence of any of (i) to (vi) by way of substitution, deletion and/or addition of one or more amino acid(s).

In certain embodiments, the wild type or unmutated F35H gene comprises
(i) a nucleotide sequence comprising the sequence of SEQ ID NO: 7;
(ii) a nucleotide sequence having the cDNA or coding sequence of SEQ ID NO: 8;
(iii) a nucleotide sequence encoding for a polypeptide having the amino acid sequence of SEQ ID NO: 9;

In certain embodiments, the wild type or unmutated F35H gene comprises
(i) a nucleotide sequence having at least 60% identity to the sequence of SEQ ID NO: 7, or 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity; or
(ii) a nucleotide sequence encoding for a polypeptide having at least 60% identity to the sequence of SEQ ID NO: 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity.

In certain embodiments, the wild type or unmutated F35H gene comprises
(i) a nucleotide sequence having at least 60% identity to the sequence of SEQ ID NO: 8; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity; or
(ii) a nucleotide sequence encoding for a polypeptide having at least 60% identity to the sequence of SEQ ID NO: 9; such as at least 65%, 70%, 75%, 80%, 85%, 90%, 92%, 94%, 95%, 96%, 97%, 98%, 99% or more sequence identity, preferably at least 85% sequence identity, more preferably at least 90% sequence identity or at least 95% sequence identity.

In certain embodiments, the wild type or unmutated F35H gene comprises
(i) a nucleotide sequence hybridizing with the reverse complement of a nucleotide sequence of SEQ ID NO: 7 or SEQ ID NO: 8, under stringent hybridization conditions.

In certain embodiments, the wild type or unmutated F35H gene comprises; or
(ii) a nucleotide sequence encoding a protein derived from the polypeptide encoded by the nucleotide sequence of SEQ ID NO: 7 or SEQ ID NO: 8 by way of substitution, deletion and/or addition of one or more amino acid(s).

The skilled person will understand that the wild type or unmutated F35H gene product is a functional gene product having enzymatic activity, as defined herein elsewhere. The skilled person will further understand that sequence variations described above for the wild type F35H do not include frame shift or nonsense mutations.

As used herein, the mutated F35H or the mutation in the F35H may comprise or may refer to any type of F35H mutation. In certain embodiments the mutation alters expression of the wild type or native F35H protein and/or mRNA. In certain embodiments the mutation reduces or eliminates expression of the (wild type or native) F35H protein and/or mRNA, as described herein elsewhere. Mutations may affect transcription and/or translation. Mutations may occur in exons or introns. Mutations may occur in regulatory elements, such as promotors, enhancers, terminators, insulators, etc. Mutations may occur in coding sequences. Mutations may occur in splicing signal sites, such as splice donor or splice acceptor sites. Mutations may be frame shift mutations. Mutations may be nonsense mutations. Mutations may be insertion or deletion of one or more nucleotides. Mutations may be non-conservative mutations (in which one or more wild type amino acids are replaced with one or more non-wild type amino acids). Mutations may affect or alter the function of the F35H protein, such as enzymatic activity. Mutations may reduce or (substantially) eliminate the function of the F35H protein, such as enzymatic activity. Reduced function, such as reduced enzymatic activity, may refer to a reduction of about at least 10%, preferably at least 30%, more preferably at least 50%, such as at least 20%, 40%, 60%, 80% or more, such as at least 85%, at least 90%, at least 95%, or more. (Substantially) eliminated function, such as (substantially) eliminated enzymatic activity, may refer to a reduction of at least 80%, preferably at least 90%, more preferably at least 95%. Mutations may be dominant negative mutations. In certain embodiments, mutations are evaluated with reference to maize inbred line PH207, as defined herein elsewhere.

In certain embodiments, the F35H mutation is an insertion of one or more nucleotides in the coding sequence. In certain embodiments, the F35H mutation is a nonsense mutation. In certain embodiments, the F35H mutation results in altered expression of the F35H gene. In certain embodiments, the F35H mutation results in knockout of the F35H gene or knockdown of the F35H mRNA and/or protein. In certain embodiments, the mutation results in a frame shift of the coding sequence of F35H. In certain embodiments, the mutation results in an altered protein sequence encoded by the F35H gene.

In certain embodiments, the F35H mutation is an insertion, preferably in an exon, preferably an insertion in the first exon, of one or more nucleotides, preferably a frame shift insertion. In certain embodiments, the insertion is 7 or 8 nucleotides. In certain embodiments, the insertion is between position 102 and 103 of the F35H gene represented by the nucleotide sequence of SEQ ID NO: 7. The skilled person is capable of determining the corresponding position in F35H homologues or orthologues. In certain embodiments, the insertion comprises or consists of the nucleotide sequence gcsgtct such as gcggtct. In certain embodiments, the insertion comprises or consists of the nucleotide sequence gcsgttct such as gcggttct. In certain embodiments, the mutated F35H comprises the nucleotide sequence of SEQ ID NO: 1 or 4, or has a cDNA or coding sequence of SEQ ID NO: 2 or 5, or encodes a protein having a sequence of SEQ ID NO: 3 or 6. Alternatively, the mutation is a substitution, preferably a substitution of at least one nucleic acid resulting in an exchange of at least one amino acid or resulting in the change of an amino acid coding codon into a stop codon.

F35H mRNA and/or protein expression may be reduced or eliminated by mutating the F35H gene itself (including coding, non-coding, and regulatory element). Methods for introducing mutations are described herein elsewhere. Alternatively, F35H mRNA and/or protein expression may be reduced or eliminated by (specifically) interfering with transcription and/or translation, such as to decrease or eliminate mRNA and/or protein transcription or translation. Alternatively, F35H mRNA and/or protein expression may be reduced or eliminated by (specifically) interfering with mRNA and/or protein stability, such as to reduce mRNA and/or protein stability. By means of example, mRNA (stability) may be reduced by means of RNAi, as described herein elsewhere. Also miRNA can be used to affect mRNA (stability). In certain embodiments, a reduced F35H expression which is achieved by reducing mRNA or protein stability is also encompassed by the term “mutated” F35H. In certain embodiments, a reduced F35H expression which is achieved by reducing mRNA or protein stability is not encompassed by the term “mutated” F35H.

In an aspect, the invention relates to the use of one or more of the polynucleic acids or QTL (allele) according to the invention described herein elsewhere for producing or generating a plant or plant part, in particular having improved (stover) digestibility.

In an aspect, the invention relates to the use of one or more of the (molecular) markers described herein for identifying a plant or plant part, in particular having improved (stover) digestibility. In an aspect, the invention relates to the use of one or more of the (molecular) markers described herein which are able to detect at least one (diagnostic) marker allele for identifying a plant or plant part, in particular having improved (stover) digestibility. In an aspect, the invention relates to the detection of one or more of the (molecular) marker (alleles) described herein for identifying a plant or plant part, in particular having improved (stover) digestibility, or for discriminating between plants or plant parts, in particular between plants and plant parts having improved (stover) digestibility and plants or plant parts not having improved (stover) digestibility.

The marker alleles of the invention as described herein may be diagnostic marker alleles which are useable for identifying and/or selecting plants or plant parts having improved digestibility, preferably improved stover digestibility.

In an aspect, the invention relates to a (isolated) polynucleic acid comprising a (molecular) marker allele of the invention, or a (unique) fragment thereof, or the complement or the reverse complement of a (molecular) marker allele of the invention, or a (unique) fragment thereof. In certain embodiments, the invention relates to a polynucleic acid comprising at least 10 contiguous nucleotides, preferably at least 15 contiguous nucleotides, more preferably at least 17 or 18 contiguous nucleotides, such as at least 20 contiguous nucleotides of a (molecular) marker (allele) of the invention, or the complement or the reverse complement of a (molecular) marker (allele) of the invention. In certain preferred embodiments, the fragment comprises at least one nucleotide of the polymorphism of the respective (molecular) marker (allele), or the complement or reverse complement thereof.

In an aspect, the invention relates to a (isolated) polynucleotide comprising a polynucleic acid having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence in a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 1 or 4, which preferably comprises gcsgtct, preferably gcggtct, at a position corresponding to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6;
or a (unique) fragment of the sequence of a) to g);
or the complement or reverse complement of the sequence of a) to g) or a (unique) fragment thereof.

In certain embodiments, the invention relates to a polynucleic acid comprising at least 10 contiguous nucleotides, preferably at least 15 contiguous nucleotides, more preferably at least 17 or 18 contiguous nucleotides, such as at least 20 contiguous nucleotides of any of SEQ ID NOs: 13 to 195, or the complement or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In certain embodiments, the invention relates to a polynucleic acid comprising at least 10 contiguous nucleotides, preferably at least 15 contiguous nucleotides, more preferably at least 17 or 18 contiguous nucleotides, such as at least 20 contiguous nucleotides of any of SEQ ID NOs: 109 to 154, or the complement or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In certain embodiments, the invention relates to a polynucleic acid comprising at least 10 contiguous nucleotides, preferably at least 15 contiguous nucleotides, more preferably at least 17 or 18 contiguous nucleotides, such as at least 20 contiguous nucleotides of any of SEQ ID NOs: 124 to 125, or the complement or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In certain embodiments, the polynucleic acid is capable of discriminating between a (molecular) marker (allele) of the invention and a non-molecular marker allele, such as to specifically hybridise with a (molecular) marker allele of the invention.

In certain embodiments, the polynucleic acid is capable of hybridising with a unique nucleotide fragment or section of any of SEQ ID NOs: 1, 2, 4, 5, 13-195, or the complement or the reverse complement of any of SEQ ID NOs: 1, 2, 4, 5, 13-195, preferably SEQ ID NOs: 1, 2, 4, 5, or 109-154, more preferably of any of SEQ ID NOs: 1, 2, 4, 5, 124, or 125, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

It will be understood that a (unique) section or fragment preferably refers to a section or fragment comprising the SNP (or at least one unique nucleotide of the polymorphism in case of for instance insertions or deletions) or the respective marker alleles of the invention (such as for instance indicated in Table A, B, or C), or a section or fragment comprising the 5′ or 3′ junction of the insert of a marker allele of the invention or a section or fraction comprised within the insert of a marker allele of the invention, or a section or fragment comprising the junction of the deletion of a marker allele of the invention. In certain embodiments, the polynucleic acid or the complement or reverse complement thereof does not (substantially) hybridise with or bind to (genomic) DNA originating from maize inbred line PH207. In certain embodiments, the sequence of the polynucleic acid or the complement or reverse complement thereof does not occur or is not present in maize inbred line PH207.

In an aspect, the invention relates to a (isolated) polynucleotide comprising or comprised in a QTL (allele), in particular associated with improved digestibility, (on chromosome 9) and comprising and/or flanked by (molecular) marker (alleles) SYN38529 and PZE-109103504, preferably PZE-109076467 and ma61161s01 or comprised in a polynucleotide or QTL (allele) comprising and/or flanked by (molecular) marker (alleles) SYN38529 and PZE-109103504, preferably PZE-109076467 and ma61161s01; the complement or reverse complement thereof; or a (unique) fragment thereof, the complement or reverse complement thereof.

In an aspect, the invention relates to a polynucleic acid capable of specifically hybridizing with a (molecular) marker allele of the invention, or the complement thereof, or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In an aspect, the invention relates to a polynucleic acid capable of specifically hybridizing with a polynucleotide having a sequence as set forth in any of SEQ ID NOs: 13-195, or the complement thereof, or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In an aspect, the invention relates to a polynucleic acid capable of specifically hybridizing with a polynucleotide having a sequence as set forth in any of SEQ ID NOs: 109-154, or the complement thereof, or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In an aspect, the invention relates to a polynucleic acid capable of specifically hybridizing with a polynucleotide having a sequence as set forth in any of SEQ ID NOs: 124-125, or the complement thereof, or the reverse complement thereof, and preferably comprising the respective corresponding polymorphism (or at least the most 5′ or most 3′ nucleotide of the polymorphism, such as in case of insertions or deletion) as listed in Table A, preferably wherein said polymorphism (or at least one nucleotide thereof) is the most 3′ nucleotide, the second most 3′ nucleotide or the third most 3′ nucleotide, preferably the most 3′ nucleotide.

In certain embodiments, the polynucleic acid is a primer. In certain embodiments, the polynucleic acid is a probe. In certain embodiments, the polynucleic acid is an allele specific primer. In certain embodiments, the polynucleic acid is KASP primer.

In certain embodiments, the polynucleic acid is an allele specific polynucleic acid, such as an allele specific primer or probe.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

In certain embodiments, the polynucleic acid comprises 15 to 500 nucleotides, preferably 17 to 100 nucleotides, more preferably 17 to 50 nucleotides, most preferably 17 to 30 nucleotides.

The skilled person will understand that the polynucleic acids, such as primers or probes, may further comprise (5′) additional nucleotides, which for instance may serve as a tag, tail, or barcode.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

In an aspect, the invention relates to a kit comprising such polynucleic acids, such as primers (comprising forward and/or reverse primers) and/or probes. The kit may further comprise instructions for use.

In will be understood that in embodiments relating to a set of forward and reverse primers, only one of both primers (forward or reverse) may need to be capable of discriminating between a (molecular) marker allele of the invention and a non-marker allele, and hence may be unique. The other primer may or may not be capable of discriminating between a (molecular) marker allele of the invention and a non-marker allele, and hence may or may not be unique.

By means of further guidance, and without limitation, the present invention is captured by the below aspects and embodiments.

In an aspect, the invention relates to a method for identifying a maize plant or plant part, comprising screening for the presence of a polynucleotide comprising (molecular) marker (allele) ma61134d15 and/or ma61134d16 in (the genome of) a maize plant or plant part.

In certain embodiments, the polynucleotide comprises or is comprised in a QTL (allele), in particular associated with improved digestibility, on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01.

In an aspect, the invention relates to a method for identifying a maize plant or plant part, comprising screening for the presence of a QTL (allele), in particular associated with improved digestibility on chromosome 9 and comprises and/or is flanked by (molecular) marker (alleles) SYN38529 and PZE-109103504, preferably PZE-109076467 and ma61161s01 or is comprised in a polynucleotide or QTL (allele) comprising and/or flanked by (molecular) marker (alleles) SYN38529 and PZE-109103504, preferably PZE-109076467 and ma61161s01.

In certain embodiments, the QTL (allele) comprises (molecular) marker (allele) ma61134d15 and/or ma61134d16.

In certain embodiments, the method comprises screening for the presence of one or more (molecular) marker (allele) selected from Table A.

In certain embodiments, the method comprises screening for the presence of one or more (molecular) marker (allele) selected from Table B.

In certain embodiments, the method comprises screening for the presence of one or more (molecular) marker (allele) selected from Table C.

In certain embodiments, the polynucleotide comprises one or more (molecular) marker (allele) as defined in statement 5.

In certain embodiments, the QTL (allele) comprises one or more (molecular) marker (allele) selected from Table A, Table B, or Table C.

In an aspect, the invention relates to a method for identifying a maize plant or plant part, comprising screening for or detecting the presence of one or more (molecular) marker (allele) selected from Table A, Table B, or Table C in (the genome of) a maize plant or plant part.

In certain embodiments, the marker is comprised in a QTL, in particular associated with improved digestibility, on chromosome 9 flanked by and/or comprising (molecular) marker (allele) SYN38529 and PZE-109103504.

In an aspect, the invention relates to a method for identifying a maize plant or plant part, comprising screening in (the genome of) a maize plant or plant part for the presence of a polynucleotide comprising a sequence selected from the group consisting of
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8.

In certain embodiments, the insertion of 8 nucleotides has a sequence of gcggttct, or wherein the insertion of 7 nucleotides has a sequence of gcggtct.

In certain embodiments, the nucleotide sequence is selected from the group consisting of
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In an aspect, the invention relates to a method for identifying a maize plant or plant part, comprising screening in (the genome of) a maize plant or plant part for the presence of a polynucleotide comprising a sequence selected from the group consisting of
a) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
b) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
c) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In certain embodiments, the method is a method for identifying a plant or plant part having improved digestibility.

In certain embodiments, the method is a method for identifying a plant or plant part having improved stover digestibility.

In certain embodiments, digestibility is improved compared to digestibility of maize line PH207 or to a (near) isogenic line which does not comprise the polynucleotide, the QTL (allele), or the one or more (molecular) marker (allele) of any of the preceding statements.

In certain embodiments, said digestibility is improved compared to digestibility of maize line PH207 or to a (near) isogenic line which does not comprise the polynucleotide, the QTL (allele), or the one or more (molecular) marker (allele) of any of the preceding statements, by at least 5%, preferably at least 10%.

In certain embodiments, the method further comprises isolating genomic DNA from the plant or plant part.

In certain embodiments, the method further comprises selecting a plant of plant part comprising the polynucleotide, one or more of the (molecular) marker (allele), or the QTL (allele).

In certain embodiments, the plant part is not propagation material.

In certain embodiments, the plant part is stover.

In certain embodiments, the plant or plant part comprises a polynucleotide having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In certain embodiments, the plant or plant part is identified as having improved digestibility if the polynucleotide, one or more of the (molecular) marker (allele), or QTL (allele) is present in the genome of said plant or plant part.

In certain embodiments, the plant or plant part is identified as having improved stover digestibility if the polynucleotide, one or more of the (molecular) marker allele, or the QTL (allele) is present in the genome of said plant or plant part.

In an aspect, the invention relates to a maize plant or plant part comprising one or more (molecular) marker (allele) as defined in Table A.

In an aspect, the invention relates to a maize plant or plant part comprising one or more (molecular) marker (allele) as defined in Table B.

In an aspect, the invention relates to a maize plant or plant part comprising one or more (molecular) marker (allele) as defined in Table C.

In an aspect, the invention relates to a maize plant of plant part comprising the polynucleotide or the QTL (allele) as described above.

In certain embodiments, the plant or plant part is derived from a plant comprising said polynucleotide, said (molecular) marker (allele) or said QTL (allele) obtained by introduction or introgression.

In certain embodiments, the plant or plant part is the result of mutagenesis mediated by transposon or transposable element.

In certain embodiments, the plant or plant part is transgenic or gene-edited.

In an aspect, the invention relates to a method for generating or producing a maize plant or plant part and/or for improving (stover) digestibility, comprising introducing into the genome of a maize plant or plant part the polynucleotide, the one or more (molecular) marker (allele), or the QTL (allele) as described above.

In an aspect, the invention relates to a method for generating or producing a maize plant or plant part and/or for improving (stover) digestibility, comprising introducing into the genome of a maize plant or plant part a polynucleotide having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In certain embodiments, introducing into the genome comprises introgression.

In certain embodiments, the method comprises the steps of (a) providing a first maize plant having the polynucleotide, the one or more (molecular) marker (allele), or the QTL (allele) as defined in any of statements 1 to 9, or obtained from a Zea mays seed as deposited under NCIMB Deposit number NCIMB 43997 (or offspring thereof), (b) crossing said first maize plant with a second maize plant, and (c) selecting progeny plants having the polynucleotide, the one or more (molecular) marker (allele), or the QTL (allele) as defined in any of statements 1 to 9.

In certain embodiments, the method further comprises (d) harvesting a plant part from said progeny.

In certain embodiments, introducing into the genome comprises mutagenesis mediated by transposon or transposable element.

In certain embodiments, introducing into the genome comprises transgenesis or gene-editing.

In certain embodiments, the plant part is a plant cell, tissue, organ, or seed.

In certain embodiments, the plant part is a an immature or mature embryo, an inflorescence, a protoplast or callus.

In certain embodiments, the method comprises the steps of transforming a plant or plant part, preferably a plant cell, more preferably an immature or mature embryo, an inflorescence, a protoplast or callus, with said polynucleotide, said one or more (molecular) marker (allele), or said QTL (allele), and optionally regenerating a plant from said plant cell, preferably immature or mature embryo, inflorescence, protoplast or callus.

In certain embodiments, the method is a method for improving digestibility of a plant or plant part.

In certain embodiments, the method is a method for improving stover digestibility.

In certain embodiments, the plant part is stover.

In an aspect, the invention relates to a maize plant or plant part obtainable by the method as described above.

In certain embodiments, the polynucleotide, said one or more (molecular) marker (allele), or said QTL (allele) is homozygous.

In certain embodiments, the polynucleotide, said one or more (molecular) marker (allele), or said QTL (allele) is heterozygous.

In an aspect, the invention relates to the use of a molecular marker as defined in Table A for identifying or selecting a maize plant or plant part.

In an aspect, the invention relates to the use of a molecular marker as defined in Table B for identifying or selecting a maize plant or plant part.

In an aspect, the invention relates to the use of a molecular marker as defined in Table C for identifying or selecting a maize plant or plant part.

In certain embodiments, the marker is used for identifying or selecting a maize plant or plant part having improved digestibility.

In certain embodiments, the marker is used for identifying or selecting a maize plant or plant part having improved stover digestibility.

In certain embodiments, digestibility is improved compared to digestibility of maize line PH207 or to a (near) isogenic line which does not comprise the polynucleotide, the QTL (allele), the one or more (molecular) marker (allele) of any of the preceding statements.

In certain embodiments, digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.

In certain embodiments, the plant part is stover.

In an aspect, the invention relates to the use of the polynucleotide or the QTL (allele) as defined herein according to the invention for generating or producing a maize plant or plant part.

In certain embodiments, the invention relates to the use of the polynucleotide or the QTL (allele) as defined herein according to the invention for generating or producing a maize plant or plant part having improved digestibility.

In certain embodiments, the invention relates to the use of the polynucleotide or the QTL (allele) as defined herein according to the invention for generating or producing a maize plant or plant part having improved stover digestibility.

In certain embodiments, digestibility is improved compared to digestibility of maize line PH207 or to a (near) isogenic line which does not comprise the polynucleotide, the QTL (allele), the one or more (molecular) marker (allele) of any of the preceding statements.

In certain embodiments digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.

In certain embodiments, the plant part is seed or stover.

In an aspect, the invention relates to a (isolated) polynucleotide comprising a polynucleic acid having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

In an aspect, the invention relates to the use of the (isolated) polynucleotide for generating or producing a maize plant or plant part.

In certain embodiments, the invention relates to the use of the (isolated) polynucleotide for generating or producing a maize plant or plant part having improved digestibility.

In certain embodiments, the invention relates to the use of the (isolated) polynucleotide for generating or producing a maize plant or plant part having improved stover digestibility.

In certain embodiments, the digestibility is improved compared to digestibility of maize line PH207 or to a (near) isogenic line which does not comprise the polynucleotide, the QTL (allele), the one or more (molecular) marker (allele) of any of the preceding statements.

In certain embodiments, the digestibility is improved compared to digestibility of maize line PH207 or to a (near) isogenic line which does not comprise the polynucleotide, the QTL (allele), the one or more (molecular) marker (allele) of any of the preceding statements, by at least 5%, preferably at least 10%.

In certain embodiments, the plant part is stover.

In an aspect, the invention relates to a (isolated) polynucleotide comprising a (molecular) marker (allele) selected from Table A, the complement or the reverse complement thereof, or a fragment thereof.

In an aspect, the invention relates to a (isolated) polynucleotide comprising a (molecular) marker (allele) selected from Table B, the complement or the reverse complement thereof, or a fragment thereof.

In an aspect, the invention relates to a (isolated) polynucleotide comprising a (molecular) marker (allele) selected from Table C, the complement or the reverse complement thereof, or a fragment thereof.

In certain embodiments, the (isolated) polynucleotide comprises 10 to 500 nucleotides, preferably 15 to 250 nucleotides, more preferably 18 to 250 nucleotides, most preferably 20 to 250 nucleotides.

In certain embodiments, the (isolated) polynucleic acid, in particular suitable as molecular marker, comprises at least 15, preferably at least 18, more preferably at least 20, contiguous nucleotides of any of SEQ ID NO: 13 to 195, or complementary to contiguous nucleotides of any of SEQ ID NO: 13 to 195, or reverse complementary to contiguous nucleotides of any of SEQ ID NO: 13 to 195, and preferably comprises at least one nucleotide of the respective polymorphism (donor allele) as provided in Table A.

In certain embodiments, the (isolated) polynucleic, in particular suitable as molecular marker, comprises at least 15, preferably at least 18, more preferably at least 20, contiguous nucleotides of any of SEQ ID NO: 109 to 154, or complementary to contiguous nucleotides of any of SEQ ID NO: 109 to 154, or reverse complementary to contiguous nucleotides of any of SEQ ID NO: 109 to 154, and preferably comprises at least one nucleotide of the respective polymorphism (donor allele) as provided in Table A.

In certain embodiments, the (isolated) polynucleic acid, in particular suitable as molecular marker, comprises at least 15, preferably at least 18, more preferably at least 20, contiguous nucleotides of any of SEQ ID NO: 124 to 125, or complementary to contiguous nucleotides of any of SEQ ID NO: 124 to 125, or reverse complementary to contiguous nucleotides of any of SEQ ID NO: 124 to 125, and preferably comprises at least one nucleotide of the respective polymorphism (donor allele) as provided in Table A.

In certain embodiments, the (isolated) polynucleotide is specific to a plant or plant part having improved stover digestibility.

In an aspect, the invention relates to a (isolated) polynucleic acid specifically hybridizing with a molecular marker of Table A, the complement or the reverse complement thereof.

In an aspect, the invention relates to a (isolated) polynucleic acid specifically hybridizing with a molecular marker of Table B, the complement or the reverse complement thereof.

In an aspect, the invention relates to a (isolated) polynucleic acid specifically hybridizing with a molecular marker of Table C, the complement or the reverse complement thereof.

In certain embodiments, the (isolated) polynucleic acid is a primer or a probe.

In certain embodiments, the (isolated) polynucleic acid is an allele-specific primer.

In certain embodiments, the (isolated) polynucleic acid is a KASP primer.

In an aspect, the invention relates to a primer or probe capable of specifically detecting the polynucleotide, the one or more (molecular) marker (allele), or the QTL (allele) of the invention as described herein elsewhere.

In an aspect, the invention relates to a primer set capable of specifically detecting the polynucleotide, the one or more (molecular) marker (allele), or the QTL (allele) of the invention as described herein elsewhere.

In an aspect, the invention relates to Zea mays seed designated K0001, a representative sample of which has been deposited under NCIMB (National Collection of Industrial Food and Marine Bacteria; Ltd. Ferguson Building, Craibstone Estate, Bucksburn, Aberdeen, AB21 9YA Scotland) Accession No. NCIMB 43997 on Jul. 6, 2022, or plants or plant parts, such as preferably stover, grown or obtained therefrom, or offspring therefrom. In an aspect, the invention relates to Zea mays seed as deposited under NCIMB Accession No. NCIMB 43997, or plants or plant parts, such as preferably stover, grown or obtained therefrom, or offspring therefrom. Seed (or plants grown therefrom or plant parts obtained (from plants grown) therefrom, or offspring therefrom) deposited under NCIMB Accession No. NCIMB 43997 comprises ma61134d16, such as defined in Table A or Table 3, detectable by molecular marker of SEQ ID NO: 124. Seed (or plants grown therefrom or plant parts obtained (from plants grown) therefrom, or offspring therefrom) deposited under NCIMB Accession No. NCIMB 43997 comprises a F35H gene (on chromosome 9) having a sequence (comprising an insertion of 7 bp) as set forth in SEQ ID NO: 4. Plants grown or obtained from seed deposited under NCIMB Accession No. NCIMB 43997, or offspring therefrom, exhibit an increased or improved stover digestibility, such as in comparison to a Zea mays plant not having said molecular marker or sequence. In an aspect, the invention relates to a plant part, such as preferably stover, obtained from or obtainable from plants grown from Zea mays seed as deposited under NCIMB Accession No. NCIMB 43997, or offspring thereof. In an aspect, the invention relates to a plant or plant part, such as preferably stover, comprising a F35H gene as present in Zea mays seed as deposited under NCIMB Accession No. NCIMB 43997, or offspring thereof. In an aspect, the invention relates to a plant or plant part, such as preferably stover, comprising a F35H gene (on chromosome 9) having a sequence (comprising an insertion of 7 bp) as set forth in SEQ ID NO: 4 as present in Zea mays seed as deposited under NCIMB Accession No. NCIMB 43997, or offspring thereof. In an aspect, the invention relates to a plant or plant part, such as preferably stover, comprising ma61134d16 (on chromosome 9), detectable by molecular marker of SEQ ID NO: 124 as present in Zea mays seed as deposited under NCIMB Accession No. NCIMB 43997, or offspring thereof. It is to be understood that when reference is made to offspring from (plants obtained from) seed deposited under NCIMB Accession No. NCIMB 43997, the recited molecular marker (ma61134d16), polynucleotide, sequence, QTL, SEQ ID NO (4 or 124), or F35H gene according to the invention (i.e. as present in deposited seed NCIMB 43997, and resulting in improved stover digestibility) is present, i.e. is at least heterozygous. The aspects and embodiments of the invention are further supported by the following non-limiting examples.

### EXAMPLES

### Example 1

The gene F35H (wildtype sequence: SEQ ID NOs: 7-9) was previously identified in as major factor on silage maize digestibility (WO 2019/206927). In this patent application, a knockout allele of the gene was described, which contains a long insertion in Exon 1 of f35h gene (SEQ ID NOs: 10-12). Furthermore, a new favorable gain of function allele identified by TILLING was described in this first patent application. Dominant and codominant KASP and KPE markers (KPE markers stand for capillary electrophoresis and show length polymorphisms. The technology was previously (and occasionally still today) used for SSR.) to detect the native insertion and the TILLING mutant were provided. These markers were applied routinely for marker assisted selection in breeding and trait integration and for diagnostic analysis of new double haploid (DH) lines for the presence of the favorable alleles. Surprisingly, a high number of lines was identified, were these markers showed contradictory data (see Table 1).

TABLE 1

| | flanking | | | | |
|---|---|---|---|---|---|
| | marker | dominant ins | dominant del | codominant | KPE |
| | ma60405s01 | ma61134d04 | ma61134d01 | ma61134d02 | ma61134xxx |
| | (SEQ ID NO: | (SEQ ID NOs: | (SEQ ID NOs: | (SEQ ID NOs: | (SEQ ID NO: |
| | 120) | 204-206) | 198-200) | 201-203) | 196) |
| non-SILO-line | ade | — | del | Del | 107 |
| (PH207) | | | | | |
| SILO-line | gua | ins | — | ins | 295 |
| new lines | gua | — | — | del | 112 or 115 |

These newly developed lines were sequenced by 40X Illumina and the gene space was assembled. Comparison of these lines with PH207 as reference for a non-SILO line and SILO lines carrying the originally identified Silo-09-02 allele showed that the ˜ 200 bp insertion was lost. However, insertions of 7-8 bp in Exon 1 off35h gene have been found. This was not excepted, because blasting of the original ˜200 bp insertion (long insertion) against the maize repeat database (http://maize.jcvi.org/repeat_db.shtml) did not reveal an annotated transposon. Markers for the 8 bp insertion and the 7 bp insertion were designed (ma61134d15; SEQ ID NO: 125 and ma61134d16; SEQ ID NO: 124).

Lines of six DH populations with the 8 bp insertion (short insertion; SEQ ID NOs: 1-3) were tested for digestible neutral detergent fiber (DNDF (cell wall digestibility)) using a NIRS calibration. The populations segregated either for wildtype F35H and 8 bp insertion mutant or for mutants with long and short insertion. In the first case the difference in digestibility was significant in the latter case, it was not, suggesting that the 8 bp insertion has the same effect on DNDF as the original transposon-like insertion. All these lines tested show the positive phenotype of improved stover digestibility (Table 2).

TABLE 2
Phenotypical analysis of six DH population with 8 bp insertion.

| | | DNDF units | | | |
|---|---|---|---|---|---|
| | | | long | 8 bp | |
| population | repeats | wildtype | Insertion | Insertion | significance |
| 1 | 6 vs 4 | 53.44 | | 61.15 | sign. difference |
| 2 | 6 vs 4 | 56.15 | | 65.71 | sign. difference |
| 3 | 8 vs 6 | 52.02 | | 58.32 | sign. difference |
| 4 | 2 vs 2 | | 65.81 | 63.25 | no difference |
| 5 | 6 vs 4 | | 61.28 | 63.78 | no difference |
| 6 | 4 vs 2 | | 61.08 | 64.57 | no difference |

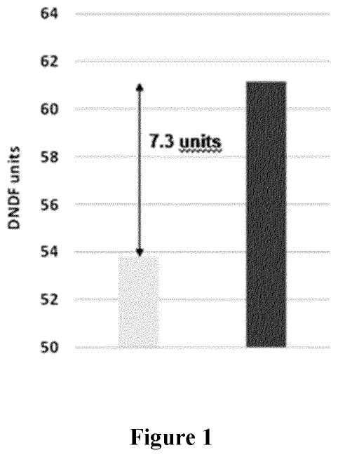

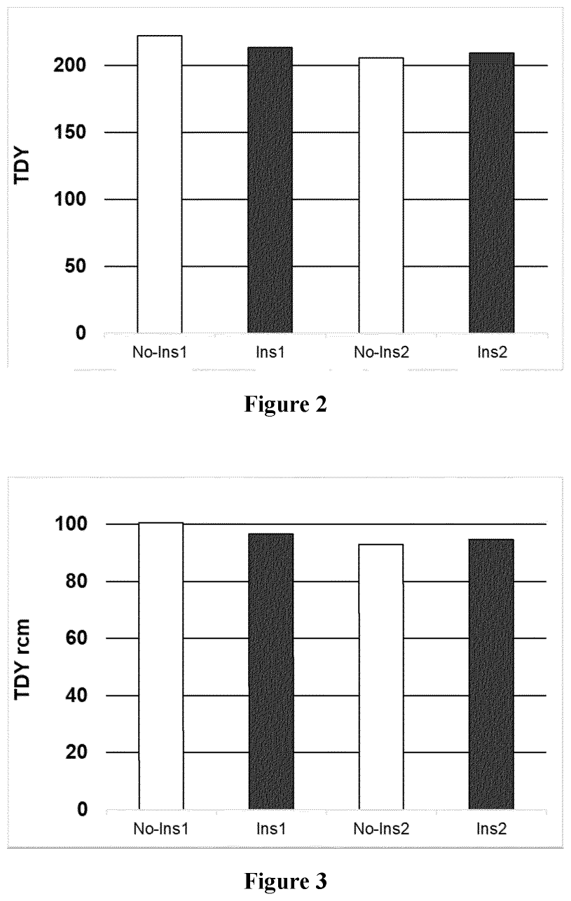

A corn line containing the 7 bp insertion (SEQ ID NOs: 4-6) was tested extensively as inbred line per se and in hybrid combination and showed high performance and high digestibility (FIG. 1).

While WO 2019/206927 lists only markers which are polymorphic between the SILO-09-02 line and the pool 5 maize, the present invention provides more markers in the area of +/−20 cM (10 cM) which are polymorphic to pool 1, pool 4 or pool 9, which are new target pools for the trait. (Table 3).

TABLE 3
Selected list of markers in a genomic region of +/−20 cM related to
map of pool 5. Markers have maximum MAF (Minor Allele Frequency)
values for use in as many other breeding pools as possible.

| SEQ | | | | | |
|---|---|---|---|---|---|
| ID | | | position | allele | |
| NO: | Marker | chromosome | [cM] | donor | Comment |
| 13 | SYN38529 | 9 | 56.38 | | polymorphic |
| 14 | SYN5866 | 9 | 57.20 | | polymorphic |
| 15 | SYN11764 | 9 | 57.72 | | polymorphic |
| 16 | PZE-109015136 | 9 | 59.42 | | polymorphic |
| 17 | PZE-109015397 | 9 | 59.62 | | polymorphic |
| 18 | PZE-109016177 | 9 | 60.98 | | polymorphic |
| 19 | PZE-109017276 | 9 | 62.41 | | polymorphic |
| 20 | PZE-109017407 | 9 | 62.60 | | polymorphic |
| 21 | PZE-109018614 | 9 | 63.85 | | polymorphic |
| 22 | SYN32168 | 9 | 64.27 | | polymorphic |
| 23 | PZE-109021152 | 9 | 65.02 | | polymorphic |
| 24 | PZE-109021389 | 9 | 65.09 | | polymorphic |
| 25 | zm06133s02 | 9 | 65.12 | | polymorphic |
| 26 | PZE-109022641 | 9 | 65.23 | | polymorphic |
| 27 | Affx-90814890 | 9 | 65.25 | | polymorphic |
| 28 | SYN34181 | 9 | 65.35 | | polymorphic |
| 29 | SYN29297 | 9 | 65.77 | | polymorphic |
| 30 | PZE-109026962 | 9 | 65.77 | | polymorphic |
| 31 | SYN13048 | 9 | 65.93 | | polymorphic |
| 32 | PZE-109063861 | 9 | 66.35 | | polymorphic |
| 33 | PZE-109029427 | 9 | 66.49 | | polymorphic |
| 34 | zm08444s04 | 9 | 66.90 | | polymorphic |
| 35 | zm08444s03 | 9 | 66.90 | | polymorphic |
| 36 | PZE-109031125 | 9 | 66.93 | | polymorphic |
| 37 | PZE-109033123 | 9 | 67.41 | | polymorphic |
| 38 | SYN18776 | 9 | 67.82 | | polymorphic |
| 39 | PZE-109035519 | 9 | 68.13 | | polymorphic |
| 40 | PZE-109035852 | 9 | 68.26 | | polymorphic |
| 41 | PZE-109036977 | 9 | 68.69 | | polymorphic |
| 42 | zm10851s01 | 9 | 68.69 | | polymorphic |
| 43 | PZE-109041138 | 9 | 69.81 | | polymorphic |
| 44 | PZE-109040213 | 9 | 70.11 | | polymorphic |
| 45 | PZE-109043990 | 9 | 70.80 | | polymorphic |
| 46 | PZE-109045856 | 9 | 71.23 | | polymorphic |
| 47 | PZE-109048885 | 9 | 71.60 | | polymorphic |
| 48 | SYN32492 | 9 | 72.05 | | polymorphic |
| 49 | SYN32488 | 9 | 72.05 | | polymorphic |
| 50 | zm11758s02 | 9 | 72.25 | | polymorphic |
| 51 | PZE-109051621 | 9 | 72.35 | | polymorphic |
| 52 | PZE-109053851 | 9 | 72.72 | | polymorphic |
| 53 | PZE-109053885 | 9 | 72.72 | | polymorphic |
| 54 | PZE-109053887 | 9 | 72.72 | | polymorphic |
| 55 | PZE-109053906 | 9 | 72.73 | | polymorphic |
| 56 | PZE-109054050 | 9 | 72.77 | | polymorphic |
| 57 | PZE-109054055 | 9 | 72.77 | | polymorphic |
| 58 | PZE-109054205 | 9 | 72.81 | | polymorphic |
| 59 | PZE-109054458 | 9 | 72.85 | | polymorphic |
| 60 | PZE-109054723 | 9 | 72.90 | | polymorphic |
| 61 | PZE-109054729 | 9 | 72.90 | | polymorphic |
| 62 | PZE-109054805 | 9 | 72.92 | | polymorphic |
| 63 | PZE-109054812 | 9 | 72.93 | | polymorphic |
| 64 | PZE-109054943 | 9 | 72.96 | | polymorphic |
| 65 | SYN32299 | 9 | 73.04 | | polymorphic |
| 66 | PZE-109058460 | 9 | 73.40 | | polymorphic |
| 67 | Affx-91355778 | 9 | 73.49 | | polymorphic |
| 68 | PZE-109061769 | 9 | 73.80 | | polymorphic |
| 69 | SYN36472 | 9 | 74.01 | | polymorphic |
| 70 | PUT-163a- | 9 | 74.08 | | polymorphic |
| | 4646495-2099 | | | | |
| 71 | PZE-109065712 | 9 | 74.20 | | polymorphic |
| 72 | Affx-91059234 | 9 | 74.24 | | polymorphic |
| 73 | PZE-109066188 | 9 | 74.28 | | polymorphic |
| 74 | PZE-109067522 | 9 | 74.36 | | polymorphic |
| 75 | PZE-109067146 | 9 | 74.43 | | polymorphic |
| 76 | PZE-109066949 | 9 | 74.44 | | polymorphic |
| 77 | PZE-109066773 | 9 | 74.47 | | polymorphic |
| 78 | PZE-100001123 | 9 | 74.50 | | polymorphic |
| 79 | PZE-100000965 | 9 | 74.52 | | polymorphic |
| 80 | ZM011231-0277 | 9 | 74.53 | | polymorphic |
| 81 | PZE-109067561 | 9 | 74.56 | | polymorphic |
| 82 | PZE-109067890 | 9 | 74.58 | | polymorphic |
| 83 | PZE-109068071 | 9 | 74.60 | | polymorphic |
| 84 | SYN21877 | 9 | 74.93 | | polymorphic |
| 85 | PZE-109071755 | 9 | 75.08 | | polymorphic |
| 86 | PZE0008250961 | 9 | 75.15 | | polymorphic |
| 87 | SYN29013 | 9 | 75.18 | | polymorphic |
| 88 | PZE-109072798 | 9 | 75.19 | | polymorphic |
| 89 | SYN23066 | 9 | 75.26 | | polymorphic |
| 90 | PZE-109073024 | 9 | 75.26 | | polymorphic |
| 91 | PZE-109073894 | 9 | 75.43 | | polymorphic |
| 92 | SYN30845 | 9 | 75.50 | | polymorphic |
| 93 | ma60054s01 | 9 | 75.50 | | polymorphic |
| 94 | PZE-109074314 | 9 | 75.51 | | polymorphic |
| 95 | SYN22542 | 9 | 75.51 | | polymorphic |
| 96 | PZE-109074671 | 9 | 75.55 | | polymorphic |
| 97 | PZE-109074676 | 9 | 75.55 | | polymorphic |
| 98 | PZE0004090171 | 9 | 75.58 | | polymorphic |
| 99 | PZE-100001246 | 9 | 75.58 | | polymorphic |
| 100 | PZE-100001337 | 9 | 75.61 | | polymorphic |
| 101 | PZE-100001338 | 9 | 75.61 | | polymorphic |
| 102 | SYN4635 | 9 | 75.65 | | polymorphic |
| 103 | PZE-109075140 | 9 | 75.66 | | polymorphic |
| 104 | PZE-109075353 | 9 | 75.70 | | polymorphic |
| 105 | PZE-109075487 | 9 | 75.72 | | polymorphic |
| 106 | PZE-109075564 | 9 | 75.74 | | polymorphic |
| 107 | PZE-109075835 | 9 | 75.78 | | polymorphic |
| 108 | PZE-109075980 | 9 | 75.79 | | polymorphic |
| 109 | PZE-109076467 | 9 | 75.85 | ade | |
| 110 | PZE-109076558 | 9 | 75.87 | gua | |
| 111 | PZE-109076739 | 9 | 75.91 | ade | |
| 112 | PZE-109076761 | 9 | 75.92 | ade | |
| 113 | PZE-109077374 | 9 | 75.97 | ade | |
| 114 | ma61126d01 | 9 | 75.98 | del | |
| 116 | PZE-109077509 | 9 | 75.98 | ade | |
| 117 | PZE-109077598 | 9 | 75.98 | gua | |
| 118 | PZE-109077976 | 9 | 76.04 | ade | |
| 119 | PZE-109078320 | 9 | 76.09 | ade | |
| 120 | ma60405s01 | 9 | 76.11 | gua | |
| 121 | PZE-109078814 | 9 | 76.16 | gua | |
| 122 | PZE-109078789 | 9 | 76.16 | cyt | |
| 123 | PZE-109079170 | 9 | 76.18 | ade | |
| 124 | ma61134d16 | 9 | 76.19 | ins | 7 bp |
| | | | | | insertion |
| 125 | ma61134d15 | 9 | 76.19 | ins | 8 bp |
| | | | | | insertion |
| 126 | Affx-91110408 | 9 | 76.27 | ade | |
| 127 | PUT-163a- | 9 | 76.28 | cyt | |
| | 16926216-1143 | | | | |
| 128 | Affx-91121762 | 9 | 76.32 | ade | |
| 129 | PZE-109080508 | 9 | 76.32 | ade | |
| 130 | PZE-109080569 | 9 | 76.32 | gua | |
| 131 | ma60182s01 | 9 | 76.35 | ade | |
| 132 | PZE-109080822 | 9 | 76.35 | ade | |
| 133 | ma61125s01 | 9 | 76.36 | ade | |
| 134 | SYN29273 | 9 | 76.41 | ade | |
| 135 | PZE-109081405 | 9 | 76.41 | gua | |
| 136 | PZE-109081479 | 9 | 76.44 | ade | |
| 137 | Affx-90937015 | 9 | 76.45 | thy | |
| 138 | PZE-109081680 | 9 | 76.46 | gua | |
| 139 | SYN29834 | 9 | 76.47 | gua | |
| 140 | PZE-109082046 | 9 | 76.50 | gua | |
| 141 | PZE-109082004 | 9 | 76.51 | gua | |
| 142 | Affx-90383113 | 9 | 76.55 | cyt | |
| 143 | SYN34099 | 9 | 76.61 | gua | |
| 144 | Affx-91186901 | 9 | 76.61 | gua | |
| 145 | SYN33659 | 9 | 76.62 | ade | |
| 146 | PZE-109082918 | 9 | 76.62 | ade | |
| 147 | PZE-109083369 | 9 | 76.67 | ade | |
| 148 | PZE-109083328 | 9 | 76.67 | gua | |
| 149 | SYN29817 | 9 | 76.75 | gua | |
| 150 | ma60360s01 | 9 | 76.91 | ade | |
| 151 | ma61158s01 | 9 | 76.91 | thy | |
| 152 | ma61036s01 | 9 | 76.95 | thy | |
| 153 | PZE-109086494 | 9 | 76.96 | cyt | |
| 154 | ma61161s01 | 9 | 77.04 | ade | |
| 155 | ma60806s03 | 9 | 77.05 | | polymorphic |
| 156 | ma61071s01 | 9 | 77.07 | | polymorphic |
| 157 | SYN38267 | 9 | 77.08 | | polymorphic |
| 158 | PZE-109088808 | 9 | 77.11 | | polymorphic |
| 159 | PUT-163a- | 9 | 77.41 | | polymorphic |
| | 60348412-2592 | | | | |
| 160 | PHM1599.84 | 9 | 77.69 | | polymorphic |
| 161 | PZE-109090188 | 9 | 77.92 | | polymorphic |
| 162 | ma60984s01 | 9 | 78.25 | | polymorphic |
| 163 | PZE-109091377 | 9 | 78.45 | | polymorphic |
| 164 | PZE-109092033 | 9 | 78.99 | | polymorphic |
| 165 | PZE-109092182 | 9 | 79.20 | | polymorphic |
| 166 | SYN27192 | 9 | 79.39 | | polymorphic |
| 167 | PZE-109093491 | 9 | 80.11 | | polymorphic |
| 168 | PZE-109093846 | 9 | 80.44 | | polymorphic |
| 169 | PZE-109094238 | 9 | 81.06 | | polymorphic |
| 170 | PZE-109094399 | 9 | 81.41 | | polymorphic |
| 171 | ZM013688-0470 | 9 | 81.72 | | polymorphic |
| 172 | PZE-109096103 | 9 | 83.49 | | polymorphic |
| 173 | SYN22829 | 9 | 83.67 | | polymorphic |
| 174 | SYN22625 | 9 | 84.92 | | polymorphic |
| 175 | PZE-109097654 | 9 | 86.06 | | polymorphic |
| 176 | PZE-109097752 | 9 | 86.10 | | polymorphic |
| 177 | PZE-109098108 | 9 | 86.63 | | polymorphic |
| 178 | zm00728s04 | 9 | 88.24 | | polymorphic |
| 179 | PZE-109098623 | 9 | 88.27 | | polymorphic |
| 180 | SYN27711 | 9 | 88.51 | | polymorphic |
| 181 | SYN27709 | 9 | 88.51 | | polymorphic |
| 182 | PZE-109099670 | 9 | 90.07 | | polymorphic |
| 183 | Affx-91229917 | 9 | 90.79 | | polymorphic |
| 184 | PZE-109100755 | 9 | 91.22 | | polymorphic |
| 185 | SYN25124 | 9 | 92.28 | | polymorphic |
| 186 | SYN25123 | 9 | 92.28 | | polymorphic |
| 187 | PZE-109101695 | 9 | 93.29 | | polymorphic |
| 188 | PZE-109101698 | 9 | 93.29 | | polymorphic |
| 189 | PZE-109102054 | 9 | 94.16 | | polymorphic |
| 190 | PZE-109102157 | 9 | 94.36 | | polymorphic |
| 191 | zm02908s02 | 9 | 94.81 | | polymorphic |
| 192 | Affx-90454668 | 9 | 94.81 | | polymorphic |
| 193 | PUT-163a- | 9 | 95.11 | | polymorphic |
| | 60357079-2822 | | | | |
| 194 | Affx-90988936 | 9 | 95.27 | | polymorphic |
| 195 | PZE-109103504 | 9 | 96.06 | | polymorphic |
| 196 | ma61134xxx | 9 | 76.19 | INS | KPE marker |
| | | | | (295 nt) | |
| 198- | ma61134d01 | 9 | 76.19 | NULL | dominant |
| 200 | | | | | wildtype |
| 201- | ma61134d02 | 9 | 76.19 | INS | codominant |
| 203 | | | | | polymorphic |
| 204- | ma61134d04 | 9 | 76.19 | INS | dominant |
| 206 | | | | | QTL allele |

1. A method for identifying a maize plant or plant part, comprising screening for the presence of or detecting a polynucleotide comprising molecular marker allele ma61134d15 and/or ma61134d16 in the genome of a maize plant or plant part; wherein ma61134d15 is an insertion of 8 nucleotides between position 134254381 and 134254382 of chromosome 9 referenced to line PH207 or at position 76.19 cM on chromosome 9 referenced to line PH207, and/or detectable by molecular marker of SEQ ID NO: 125; preferably an insertion as set forth in SEQ ID NO: 1, and ma61134d16 is an insertion of 7 nucleotides between position 134254381 and 134254382 of chromosome 9 referenced to line PH207 or at position 76.19 cM on chromosome 9 referenced to line PH207, and/or detectable by molecular marker of SEQ ID NO: 124; preferably an insertion as set forth in SEQ ID NO: 4.

2. The method according to claim 1, comprising screening for the presence of one or more molecular marker allele selected from Table B or C.

3. A maize plant or plant part comprising in the genome of a maize plant or plant part a polynucleotide having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

4. The plant or plant part according to claim 3, wherein said plant or plant part is derived from a plant comprising said polynucleotide obtained by introduction or introgression, or wherein said plant or plant part is mutagenized mediated by transposon or transposable element, is transgenic or gene-edited.

5. A method for generating or producing a maize plant or plant part and/or for improving stover digestibility, comprising introducing into the genome of a maize plant or plant part the polynucleotide as defined claim 1.

6. The method according to claim 5, comprising introducing into the genome of a maize plant or plant part a polynucleotide having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

7. The method according to claim 5, comprising (a) providing a first maize plant having the polynucleotide, or the one or more molecular marker allele, or a first plant obtained from a Zea mays seed as deposited under NCIMB Deposit number NCIMB 43997 (or offspring thereof), (b) crossing said first maize plant with a second maize plant, and (c) selecting progeny plants having the polynucleotide, or the one or more molecular marker allele.

8. The method according to claim 5, wherein introducing includes stable or transient integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cas12), CRISPR/CasX, or CRISPR/CasY), TALENs, zinc finger nucleases or meganucleases, homologous recombination optionally by means of one of the gene editing technology including preferably a repair template, modification of endogenous gene using random or targeted mutagenesis like TILLING or above mentioned gene editing technology or mutagenesis mediated by transposon or transposable element.

9. A maize plant or plant part obtainable by the method according to claim 5.

10. A method of using a molecular marker as defined in claim 1, the method comprising identifying or selecting a maize plant or plant part.

11. A method of using the polynucleotide as defined in claim 6, the method comprising generating or producing a maize plant or plant part.

12. An isolated polynucleotide comprising a polynucleic acid having a sequence selected from
a) a nucleotide sequence of SEQ ID NO: 7;
b) a nucleotide sequence having a coding sequence of SEQ ID NO: 8;
c) a nucleotide sequence which is at least 90% identical to SEQ ID NO: 7 or 8;
d) a nucleotide sequence encoding an amino acid sequence of SEQ ID NO: 9 or an amino acid sequence which is at least 90% identical to the sequence of SEQ ID NO: 9;
wherein the nucleotide sequence of a) to d) has an insertion of one or more nucleotides, preferably not a multiple of three, more preferably of 8 or 7 nucleotides at a position corresponding to the position between thymine at position 102 and guanine at position 103 of SEQ ID NO: 7 or the corresponding position in SEQ ID NO: 8; or
e) a nucleotide sequence of SEQ ID NO: 1 or 4 or a nucleotide sequence which is at least 90% identical to SEQ ID NO: 1 or 4, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
f) a nucleotide sequence having a coding sequence of SEQ ID NO: 2 or 5 or a nucleotide sequence which is at least 90% identical to SEQ ID NO: 2 or 5, which preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1;
g) a nucleotide sequence encoding an amino acid sequence of SEQ ID NO: 3 or 6 or an amino acid sequence which is at least 90% identical to the sequence of SEQ ID NO: 3 or 6, which nucleotide sequence preferably comprises gcsgtct, preferably gcggtct, at a position corresponding respectively to position 103-109 of SEQ ID NO: 4, or gcsgttct, preferably gcggttct, at a position corresponding to position 103-110 of SEQ ID NO: 1.

13. An isolated polynucleotide comprising a molecular marker allele as defined in claim 1, the complement or the reverse complement thereof, or a fragment thereof; or specifically hybridizing with a molecular marker as defined in claim 1, the complement or the reverse complement thereof, or a fragment thereof.

14. The isolated polynucleic acid according to claim 13, in particular suitable as molecular marker, comprising at least 15, preferably at least 18, more preferably at least 20, contiguous nucleotides of any of SEQ ID NO: 109 to 154, or complementary to contiguous nucleotides of any of SEQ ID NO: 109 to 154, or reverse complementary to contiguous nucleotides of any of SEQ ID NO: 109 to 154, and preferably comprising at least one nucleotide of the respective polymorphism as provided in Table B or C.

15. The isolated polynucleic acid according to claim 13, which is a primer or a probe, preferably an allele-specific primer, more preferably a KASP primer.

16. A Zea mays seed as deposited under NCIMB Deposit number NCIMB 43997.

17. A Zea mays plant grown or obtained from the seed according to claim 16, or offspring therefrom.

18. A Zea mays plant part obtained from the plant according to claim 17.

19. The Zea mays plant part according to claim 18, wherein said plant part is stover.
